(19) United States
(12) Patent Application Publication
(10) Pub. No.: US 2020/0199174 A1
(43) Pub. Date: Jun. 25, 2020

(54) NOVEL FLUORESCENT LABELING METHOD

(71) Applicant: The University of Tokyo, Tokyo (JP)

(72) Inventors: Kenzo HIROSE, Tokyo (JP); Daisuke ASANUMA, Tokyo (JP); Shigeyuki NAMIKI, Tokyo (JP); Rieko TANAKA, Tokyo (JP)

(21) Appl. No.: 16/479,186

(22) PCT Filed: Jan. 18, 2019
(86) PCT No.: PCT/JP2018/001457

§ 371 Date: Jan. 13, 2020

(30) Foreign Application Priority Data
Jan. 19, 2017 (JP) 2017-007952
Sep. 13, 2017 (JP) 2017-175980

(51) Int. Cl. C07K 1/13; C07K 16/44; C12Q 1/02; C07K 19/00

(52) U.S. Cl. CPC C07K 1/13; C07K 19/00; C12Q 1/02; C07K 16/44

(57) ABSTRACT

A method for fluorescently labeling an intracellular protein through use of a fluorescence ON/OFF control technique includes intracellularly obtaining a fusion protein of a protein to be labeled and an anti-DNP antibody, bringing a compound represented by

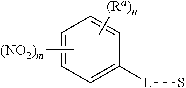

or its salt into contact with the cell, and fluorescently labeling the protein to be labeled by reacting the fusion protein and the compound or its salt.

### TECHNICAL FIELD

The present invention relates to a novel method for fluorescently labeling an intracellular protein, and to an anti-DNP antibody and a fluorescent probe used in the method.

### BACKGROUND ART

Fluorescent imaging techniques that make it possible to track, in real time, the distribution of a functional molecule in a cell as the distribution thereof develops over time are effective means for understanding molecular mechanisms on which cellular function is based. Visualization analysis using a fusion protein in which the fluorescent protein GFP is introduced by genetic engineering into a protein to be analyzed has come to be widely used in analysis of protein dynamics in cells (Non-patent Document 1).

Because the protein GFP has a relatively small molecular weight of 27 kDa and does not require an external substrate to emit fluorescence, and thus is suitable for convenient fluorescent labeling of an object protein in a cell, GFP has been tried in various fluorescent labeling applications. By causing cells to express a fusion protein of GFP and a protein to be analyzed, and analyzing the localization or dynamics of the expressed fusion protein, functions of transcription factors, cytoskeletal molecules, receptors, and various other molecules have been further elucidated (Non-patent Documents 2 and 3).

In recent years, progress has been made with molecular tagging techniques useful for fluorescent imaging by approaches that introduce chemical biological methods. A Halo-tag protein has been developed by genetically modifying a bacterial haloalkane dehalogenase enzyme (Non-patent Document 4).

Almost at the same time, a SNAP-tag protein was also developed by modifying the DNA repair enzyme 06-alkylguanine-DNA alkyl-transferase (Non-patent Document 5).

In these tagging techniques, a fluorescent ligand that is specific to the Halo-tag protein or the SNAP-tag protein covalently bonds thereto, and fluorescent labeling that is specific to a target molecule is thereby possible. For example, super-resolution imaging in a chemically fixed specimen and a living cell using a photoactivated dye that binds to the Halo-tag protein has been reported (Non-patent Document 6), and multimerization of proteins has been measured by fluorescence resonance energy transfer (FRET) using a fluorescent dye labeled via a SNAP-tag (Non-patent Document 7). A protein labeling method referred to as ligand-directed tosyl chemistry has also been reported (Non-patent Document 8). In this method, a small-molecule ligand having affinity for a specific protein binds to a target protein, whereby a reaction occurs between a tosyl group covalently bonded to the ligand and an amino acid residue near an active center of the protein, the ligand is cut off, and the target protein is labeled only with a probe portion.

The fluorescent probes used in Halo-tagging or SNAP-tagging and other molecular tagging techniques are constitutively fluorescent, and fluorescence originating from the fluorescent probe non-specifically bound to a specimen or unlabeled fluorescent molecules present outside a cell is therefore observed as a background signal, which is a factor that impedes good-contrast microscope observation of a molecule to be observed. In order to decrease this background signal, after the molecule to be observed is fluorescently labeled, washing must be performed and unreacted fluorescent probe must be removed. However, washing of biological molecules present in a cell or in a living body is generally difficult, and often cannot be performed.

A fluorescence ON/OFF control technique, whereby a fluorescent probe that is not bound to a target molecule is nonfluorescent (fluorescence OFF) and the fluorescent probe becomes fluorescent (fluorescence ON) only upon binding to the target molecule, has the potential to overcome the foregoing problem. Detection of rRNA to which an RNA aptamer sequence is added has been shown to be possible by utilizing the property of several nonfluorescent dyes whereby fluorescence thereof is turned ON by binding of the dye with a nucleic acid (RNA) aptamer (Non-patent Documents 9 and 10).

However, a practically usable fluorescence ON/OFF control technique has not yet been developed.

Super-resolution microscope techniques for realizing nanoscale spatial resolution not restricted by the diffraction limit of light have also been developed in recent years, and have rapidly advanced (Non-patent Document 11). In a super-resolution microscope technique, a super-resolution image obtained by single-molecule localization microscopy is acquired by acquiring position information of a fluorescent dye under a condition of fluorescence intermittency. Specifically, an operation in which only a small number of fluorescent molecules in a measuring field are caused to fluoresce stochastically, and the center of the location of fluorescence is determined with a precision of several tens of nanometers is repeated, and approximately 10,000 images are reconstructed to obtain a super-resolution image.

Thiol- and light-dependent photoswitching of cyanine dyes is used to cause fluorescence intermittency in single-molecule localization microscopy (Non-patent Document 12), but a thiol compound must be used as a reducing agent in this case. Due to cytotoxicity, the thiol compound is difficult to apply in a living cell, and there is therefore a need for a super-resolution imaging technique whereby fluorescence intermittency can be obtained without use of a cytotoxic reducing agent or the like. However, such a technique has not yet been practically developed.

### PRIOR ART DOCUMENTS

### Non-Patent Documents

Non-patent document 1: D. M. Chudakov, S. Lukyanov, K. A. Lukyanov, Fluorescent proteins as a toolkit for in vivo imaging. Trends in Biotechnology 23, 605-613 (2005).
Non-patent document 2: A. Miyawaki, Fluorescence imaging of physiological activity in complex systems using GFP-based probes. Current Opinion in Neurobiology 13, 591-596 (2003).
Non-patent document 3: R. Y. Tsien, The green fluorescent protein. Annual review of biochemistry 67, 509-544 (1998).
Non-patent document 4: G. V. Los et al., HaloTag: a novel protein labeling technology for cell imaging and protein analysis. ACS chemical biology 3, 373-382 (2008).
Non-patent document 5: T. Gronemeyer, C. Chidley, A. Juillerat, C. Heinis, K. Johnsson, Directed evolution of O6-alkylguanine-DNA alkyltransferase for applications in protein labeling. Protein engineering, design & selection: PEDS 19, 309-316 (2006).
Non-patent document 6: H. L. Lee et al., Superresolution imaging of targeted proteins in fixed and living cells using photoactivatable organic fluorophores. Journal of the American Chemical Society 132, 15099-15101 (2010).
Non-patent document 7: D. Maurel et al., Cell-surface protein-protein interaction analysis with time-resolved FRET and snap-tag technologies: application to GPCR oligomerization. Nat Methods 5, 561-567 (2008).
Non-patent document 8: S. Tsukiji, M. Miyagawa, Y. Takaoka, T. Tamura, I. Hamachi, Ligand-directed tosyl chemistry for protein labeling in vivo. Nature chemical biology 5, 341-343 (2009).
Non-patent document 9: J. S. Paige, K. Y. Wu, S. R. Jaffrey, RNA mimics of green fluorescent protein. Science 333, 642-646 (2011).
Non-patent document 10: R. L. Strack, M. D. Disney, S. R. Jaffrey, A superfolding Spinach2 reveals the dynamic nature of trinucleotide repeat-containing RNA. Nat Methods 10, 1219-1224 (2013).
Non-patent document 11: Huang, B. et al., Cell 143, 1047 (2010)
Non-patent document 12: Dempsey, G. T., et al. J. Am. Chem. Soc. 131, 18192 (2009)
Non-patent document 13: M. J. Rust, M. Bates, X. Zhuang, Sub-diffraction-limit imaging by stochastic optical reconstruction microscopy (STORM). Nat Methods 3, 793-795 (2006).
Non-patent document 14: M. Heilemann, S. van de Linde, M. Schuettpelz, R. Kasper, B. Seefeldt, A. Mukherjee, P. Tinnefeld, M. Sauer, Subdiffraction-resolution fluorescence imaging with conventional fluorescent probes. Angew Chem Int Ed Engl 47, 6172-6176 (2008).

### SUMMARY OF THE INVENTION

### Problems to be Solved by the Invention

An object of the present invention is to provide a novel method for fluorescently labeling an intracellular protein through use of a fluorescence ON/OFF control technique.

An object of the present invention is also to provide an antibody and a fluorescent probe that can be suitably used in the abovementioned fluorescent labeling method.

An object of the present invention is also to provide a super-resolution imaging technique which uses the abovementioned fluorescent labeling method.

### Means Used to Solve the Above-Mentioned Problems

For the purpose of developing a molecular tagging technique provided with a function for controlling an ON/OFF state of fluorescence in a cell, the inventors utilized a quenching phenomenon which occurs when a fluorescent dye is brought into proximity with a group of atoms (quencher) having fluorescence quenching ability in order to control the ON/OFF state of fluorescence. Here, with the basic principle of a fluorescence ON/OFF control technique being that the quenching phenomenon is removed and fluorescence is turned ON (fluorescent) by binding of a quencher and an anti-quencher antibody, the inventors discovered as a result of concentrated investigation that an excellent fluorescence ON/OFF control technique can be provided by controlling ability to quench a fluorescent substance using an anti-DNP (dinitrophenyl compound) antibody, and thus accomplished the present invention.

The inventors also discovered that super-resolution imaging of high commercial viability can be realized by controlling binding/dissociation kinetics of a fluorescent probe in which fluorescence is OFF (quenched organic dye emission probe (QODE)) and a molecular tag (de-quenching of organic dye emission tag (De-QODE tag)) in which quenching is removed and fluorescence is turned ON by binding of the molecular tag with an anti-quencher antibody.

Specifically, the present invention provides the following.

[1] A method for fluorescently labeling an intracellular protein, said method comprising:

obtaining, in a cell, a fusion protein of a labeling object protein and an anti-DNP (dinitrophenyl compound) antibody;

bringing a compound represented by formula (I) or a salt thereof into contact with the cell; and

fluorescently labeling the object protein by reacting the fusion protein and the compound represented by formula (I) or a salt thereof.

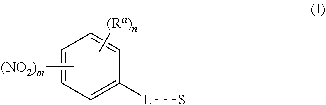

(In the formula (I):

S is a fluorescent group,

L is a linker, and

R^{a} is a monovalent substituent;

m is an integer of 0 to 2, and

n is an integer of 0 to 2;

when m is 2, n is 0;

when m is 1, n is 1 or 0;

when m is 0, n is 2; and

when n is 2, the monovalent substituents of R^{a} may be the same or different.)

[2] The method according to [1], wherein the monovalent substituent represented by R^{a} is selected from the group consisting of a halogen atom, a C1-10 alkyl group, a C1-10 alkoxy group, a cyano group, an ester group, an amide group, an alkyl sulfonyl group, a C1-10 alkyl group in which at least one hydrogen atom is substituted with a fluorine atom, and a C1-10 alkoxy group in which at least one hydrogen atom is substituted with a fluorine atom.

[3] A method for fluorescently labeling an intracellular protein, said method comprising:

obtaining, in a cell, a fusion protein of a labeling object protein and an anti-DNP (dinitrophenyl compound) antibody;

bringing a compound represented by formula (Ia) or a salt thereof into contact with the cell, and

fluorescently labeling the object protein by reacting the fusion protein and the compound represented by formula (Ia) or a salt thereof.

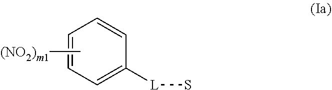

(In formula (1a):

S is a fluorescent group,

L is a linker, and

m1 is 1 or 2.)

[4] The method according to any one of [1] to [3], wherein:

the anti-DNP antibody in the fusion protein is an anti-DNP antibody or an antigen-binding fragment thereof comprising

a light chain including a VL-CDR1 comprising the amino acid sequence represented by SEQ ID NO: 1, a VL-CDR2 comprising the amino acid sequence represented by SEQ ID NO: 2, and a VL-CDR3 comprising the amino acid sequence represented by SEQ ID NO: 3, and

a heavy chain including a VH-CDR1 comprising the amino acid sequence represented by SEQ ID NO: 4, a VH-CDR2 comprising the amino acid sequence represented by SEQ ID NO: 5, and a VH-CDR3 comprising the amino acid sequence represented by SEQ ID NO: 6.

Sequence No. 1:
QEISGY
Sequence No. 2:
AAS
Sequence No. 3:
VQYASYPYT
Sequence No. 4:
GFTFSNYWMNW
Sequence No. 5:
IRLKSNNYAT
Sequence No. 6:
TGYYYDSRYGY

[5] The method according to [4], wherein the anti-DNP antibody or antigen-binding fragment thereof is a single-chain Fv (scFv).

[6] The method according to [4] or [5], wherein the anti-DNP antibody comprises an amino acid sequence having at least 90% homology to the amino acids of SEQ ID NO: 7, and includes amino acid sequences represented by SEQ ID NO: 1 to 6.

Sequence No. 7:
MADYKDIVLTQSPSSLSASLGERVSLTCRSSQEISGYLGWLQQKPDGSIK
RLIYAASTLDSGVPKRFSGSRSGSDYSLTISSLESEDFADYYCVQYASYP
YTFGGGTKLEMKRGGGGSGGGGSGGGGSGGGGSQIQLQESGGGLVQPGGS
MKLSCVASGFTFSNYWMNWVRQSPEKGLDWVAEIRLKSNNYATHYAESVK
GRFTISRDDSKSSVYLQMNNLRAEDTGIYYCTGYYYDSRYGYWGQGTTVT
VSS

[7] The method according to [6], wherein the amino acid sequence is SEQ ID NO: 7.

[8] The method according to any one of [1] to [3], wherein the anti-DNP antibody in the fusion protein comprises an amino acid sequence having at least 90% homology to the amino acids of SEQ ID NO: 7 and includes the amino acid sequences represented by SEQ ID NO: 1 to 6,

and comprises an amino acid sequence in which at least one of substitutions below is made in the amino acid sequence represented by any of SEQ ID NO: 1 to 6:

(a) any one amino acid from among glutamic acid at position 33, tyrosine at position 37, valine at position 94, glutamine at position 95, glycine at position 159, phenylalanine at position 160, phenylalanine at position 162, asparagine at position 164, glycine at position 233, tyrosine at position 235, tyrosine at position 236, aspartic acid at position 237, arginine at position 239, tyrosine at position 240, and tyrosine at position 242 numbered from an N-terminus is substituted with alanine; or

(b) any one amino acid from among tyrosine at position 96 and tyrosine at position 234 numbered from the N-terminus is substituted with phenylalanine.

[9] The method according to any one of [1] to [3], wherein the anti-DNP antibody in the fusion protein comprises an amino acid sequence in which a substitution below is made in the amino acids of SEQ ID NO: 7:

(1) any one amino acid from among glutamic acid at position 33, tyrosine at position 37, valine at position 94, glutamine at position 95, glycine at position 159, phenylalanine at position 160, phenylalanine at position 162, asparagine at position 164, glycine at position 233, tyrosine at position 235, tyrosine at position 236, aspartic acid at position 237, arginine at position 239, tyrosine at position 240, and tyrosine at position 242 numbered from the N-terminus is substituted with alanine; or

(2) any one amino acid from among tyrosine at position 96 and tyrosine at position 234 numbered from the N-terminus is substituted with phenylalanine.

[10] The method according to any one of [1] to [9], wherein obtaining the fusion protein includes obtaining a polynucleotide coding for the fusion protein, obtaining a plasmid or vector capable of expressing the fusion protein, causing the fusion protein to be expressed in a cell, or isolating the expressed fusion protein.

[11] The method according to any one of [1] to [10], wherein the linker is represented by T-Y, where Y represents a bonding group for bonding with the fluorescent group S, and T represents a crosslinking group.

[12] The method according to [11], wherein the bonding group is selected from an amide group, an alkylamide group, a carbonylamino group, an ester group, an alkylester group, or an alkylether group.

[13] The method according to any one of [1] to [12], wherein S is represented by formula (II) below.

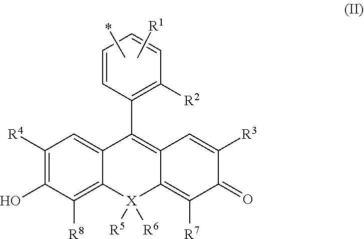

(In formula (II): R¹ represents a hydrogen atom or one to four same or different monovalent substituents which are present on a benzene ring;

R² represents a hydrogen atom, a monovalent substituent, or a bond;

R³ and R⁴ each independently represent a hydrogen atom, a C1-6 alkyl group, or a halogen atom;
R⁵ and R⁶ each independently represent a C1-6 alkyl group, an aryl group, or a bond, provided that R⁵ and R⁶ being absent when X is an oxygen atom;

R⁷ and R⁸ each independently represent a hydrogen atom, a C1-6 alkyl group, a halogen atom, or a bond;

X represents an oxygen atom or a silicon atom; and

* represents a location of bonding with L in formula (I) at any position on the benzene ring.)

[14] The method according to any one of [1] to [12], wherein S is represented by formula (III) below.

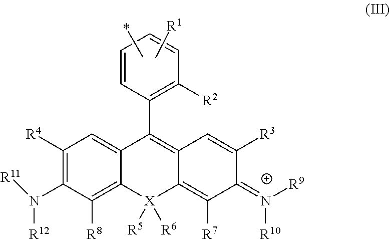

(In formula (III): R¹ to R⁸ and X are as defined in formula (II);

R⁹ and R¹⁰ each independently represent a hydrogen atom or a C1-6 alkyl group;

R⁹ and R¹⁰ may also together form a 4- to 7-membered heterocyclyl which includes a nitrogen atom to which R⁹ and R¹⁰ are bonded;

either R⁹ or R¹⁰, or both R⁹ and R¹⁰ may also respectively combine with R³ or R⁷ to form a 5- to 7-membered heterocyclyl or heteroaryl which includes a nitrogen atom to which R⁹ or R⁰ is bonded, and may comprise one to three additional hetero atoms selected from the group consisting of an oxygen atom, a nitrogen atom, and a sulfur atom as ring-forming members, and the heterocyclyl or heteroaryl may be furthermore substituted with a C1-6 alkyl, a C2-6 alkenyl, or a C2-6 alkynyl, a C6-10 aralkyl group, or a C6-10 alkyl-substituted alkenyl group;

R¹¹ and R² each independently represent a hydrogen atom or a C1-6 alkyl group;

R¹¹ and R¹² may also together form a 4- to 7-membered heterocyclyl which includes a nitrogen atom to which R¹¹ and R¹² are bonded;

either R¹ or R¹², or both R¹¹ and R^{:2} may also respectively combine with R⁴ or R⁸ to form a 5- to 7-membered heterocyclyl or heteroaryl which includes a nitrogen atom to which R¹¹ or R¹² is bonded, and may comprise one to three additional hetero atoms selected from the group consisting of an oxygen atom, a nitrogen atom, and a sulfur atom as ring-forming members, and the heterocyclyl or heteroaryl may be furthermore substituted with a C1-6 alkyl, a C2-6 alkenyl, or a C2-6 alkynyl, a C6-10 aralkyl group, or a C6-10 alkyl-substituted alkenyl group; and

* represents a location of bonding with L in formula (I) at any position on the benzene ring.)

[15] An anti-DNP antibody or an antigen-binding fragment thereof comprising:

a light chain including a VL-CDR1 comprising the amino acid sequence represented by SEQ ID NO: 1, a VL-CDR2 comprising the amino acid sequence represented by SEQ ID NO: 2, and a VL-CDR3 comprising the amino acid sequence represented by SEQ ID NO: 3; and

a heavy chain including a VH-CDR1 comprising the amino acid sequence represented by SEQ ID NO: 4, a VH-CDR2 comprising the amino acid sequence represented by SEQ ID NO: 5, and a VH-CDR3 comprising the amino acid sequence represented by SEQ ID NO: 6.

Sequence No. 1:
QEISGY
Sequence No. 2:
AAS
Sequence No. 3:
VQASYPYT
Sequence No. 4:
GFTFSNYWMNW
Sequence No. 5:
IRLKSNNYAT
Sequence No. 6:
TGYYYDSRYGY

[16] The anti-DNP antibody or antigen-binding fragment thereof according to [15], wherein the anti-DNP antibody or antigen-binding fragment thereof is a single-chain Fv (scFv).

[17] The anti-DNP antibody or antigen-binding fragment thereof according to [15] or [16], comprising an amino acid sequence having at least 90% homology to SEQ ID NO: 7 and including amino acid sequences represented by SEQ ID NO: 1 to 6.

Sequence No. 7:
MADYKDIVLTQSPSSLSASLGERVSLTCRSSQEISGYLGWLQQKPDGSIK
RLIYAASTLDSGVPKRFSGSRSGSDYSLTISSLESEDFADYYCVQYASYP
YTFGGGTKLEMKRGGGGSGGGGSGGGGSGGGGSQIQLQESGGGLVQPGGS
MKLSCVASGFTFSNYWMNWVRQSPEKGLDWVAEIRLKSNNYATHYAESVK
GRFTISRDDSKSSVYLQMNNLRAEDTGIYYCTGYYYDSRYGYWGQGTTVT
VSS

[18] The anti-DNP antibody or antigen-binding fragment thereof according to [17], wherein the amino acid sequence is SEQ ID NO: 7.

[19] An anti-DNP antibody or an antigen-binding fragment thereof, comprising an amino acid sequence having at least 90% homology to the amino acids of SEQ ID NO: 7 and including the amino acid sequences represented by SEQ ID NO: 1 to 6, and comprising an amino acid sequence in which at least one of substitutions below is made in the amino acid sequence represented by any of SEQ ID NO: 1 to 6:

(a) any one amino acid from among glutamic acid at position 33, tyrosine at position 37, valine at position 94, glutamine at position 95, glycine at position 159, phenylalanine at position 160, phenylalanine at position 162, asparagine at position 164, glycine at position 233, tyrosine at position 235, tyrosine at position 236, aspartic acid at position 237, arginine at position 239, tyrosine at position 240, and tyrosine at position 242 numbered from the N-terminus is substituted with alanine; or

(b) any one amino acid from among tyrosine at position 96 and tyrosine at position 234 numbered from the N-terminus is substituted with phenylalanine.

[20] An anti-DNP antibody or an antigen-binding fragment thereof, comprising an amino acid sequence in which a substitution below is made in the amino acids of SEQ ID NO: 7:

(1) any one amino acid from among glutamic acid at position 33, tyrosine at position 37, valine at position 94, glutamine at position 95, glycine at position 159, phenylalanine at position 160, phenylalanine at position 162, asparagine at position 164, glycine at position 233, tyrosine at position 235, tyrosine at position 236, aspartic acid at position 237, arginine at position 239, tyrosine at position 240, and tyrosine at position 242 numbered from the N-terminus is substituted with alanine; or

(2) any one amino acid from among tyrosine at position 96 and tyrosine at position 234 numbered from the N-terminus is substituted with phenylalanine.

[21] An isolated nucleic acid coding for the antibody or antigen-binding fragment thereof according to any one of [15] through [18].

[22] The nucleic acid according [21], comprising a base sequence represented by SEQ ID NO: 8.

Sequence No. 8:
ATGGCGGACTACAAAGACATTGTGCTGACCCAGTCTCCATCCTCTTTATC
TGCCTCTCTGGGAGAAAGAGTCAGTCTCACTTGTCGGTCAAGTCAGGAAA
TTAGTGGTTACTTAGGCTGGCTTCAGCAGAAACCAGATGGAAGTATTAAA
CGCCTGATCTACGCCGCATCCACTTTAGATTCTGGTGTCCCAAAAAGGTT
CAGTGGCAGTAGGTCTGGGTCAGATTATTCTCTCACCATCAGCAGCCTTG
AGTCTGAAGATTTTGCAGACTATTATTGTGTACAATATGCTAGTTATCCG
TACACGTTCGGAGGGGGGACCAAGCTGGAAATGAAACGCGGTGGTGGTGG
TTCTGGTGGTGGTGGTTCTGGCGGCGGCGGCTCCGGTGGTGGTGGATCCC
AGATTCAGCTTCAGGAGTCTGGAGGAGGCTTGGTGCAACCTGGAGGATCC
ATGAAACTCTCCTGTGTTGCCTCTGGATTCACTTTCAGTAACTACTGGAT
GAACTGGGTCCGCCAGTCTCCAGAGAAGGGGCTTGACTGGGTTGCTGAAA
TTAGATTGAAATCTAATAATTATGCAACACATTATGCGGAGTCTGTGAAA
GGGAGGTTCACCATCTCAAGAGATGATTCCAAAAGTAGTGTCTACCTGCA
AATGAACAACTTAAGAGCTGAAGACACTGGCATTTATTACTGTACCGGTT
ATTACTACGATAGTAGGTACGGCTACTGGGGCCAAGGCACCACGGTCACC
GTCTCCTCGGCCTCG

[23] An isolated nucleic acid coding for the antibody or antigen-binding fragment according to [20] or [21].

[24] A plasmid or vector including the nucleic acid according to any one of [21] to [23].

[25] A fluorescent probe used in the method according to any one of [1] to [10], comprising the compound represented by formula (I) or a salt thereof.

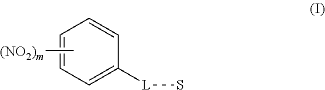

(In the formula (I):

S is a fluorescent group,

L is a linker, and

m is an integer of 1 or 2.)

[26] The fluorescent probe according to [25], used for in vivo imaging.

[27] A compound represented by a formula below, or a salt thereof.

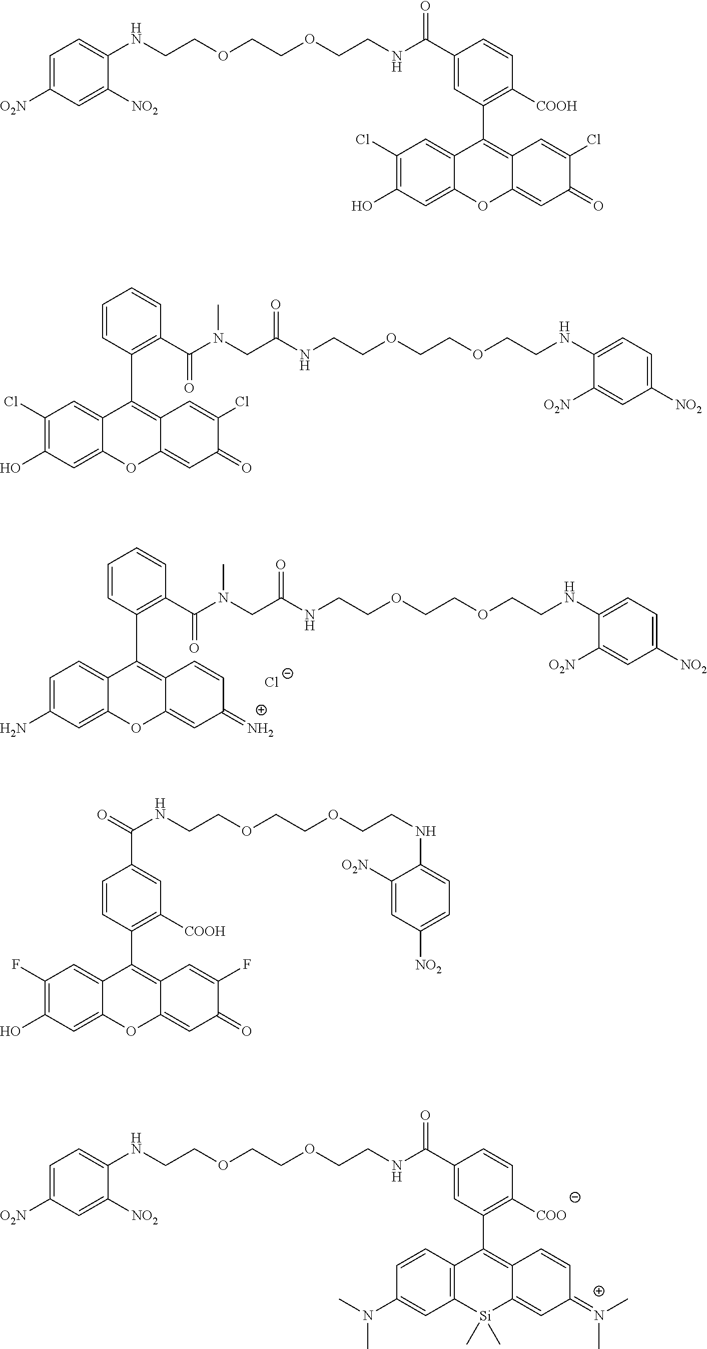

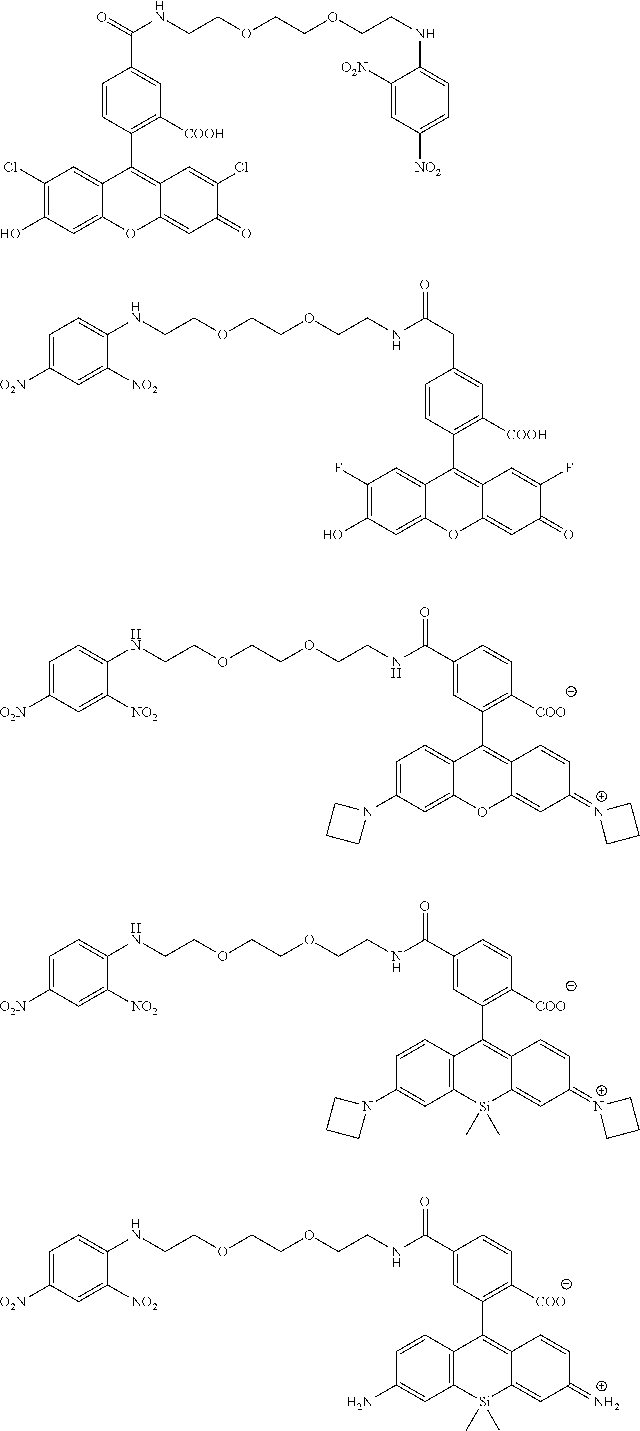

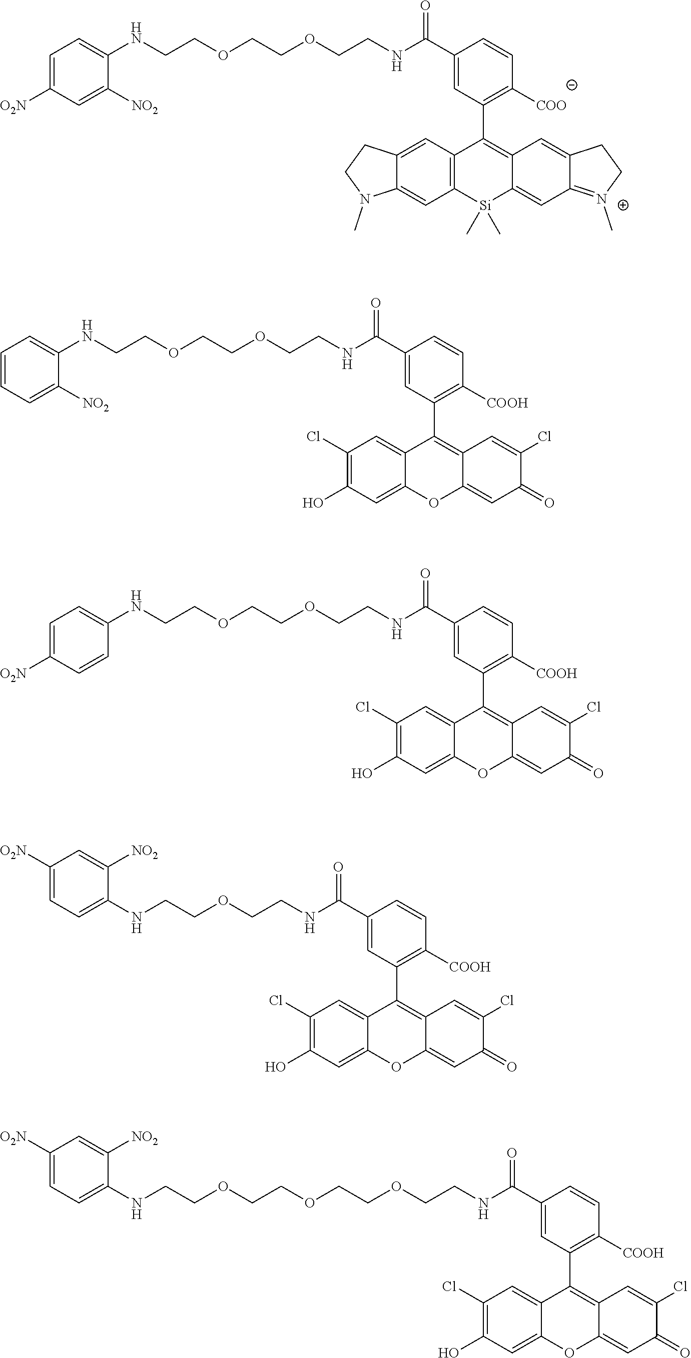

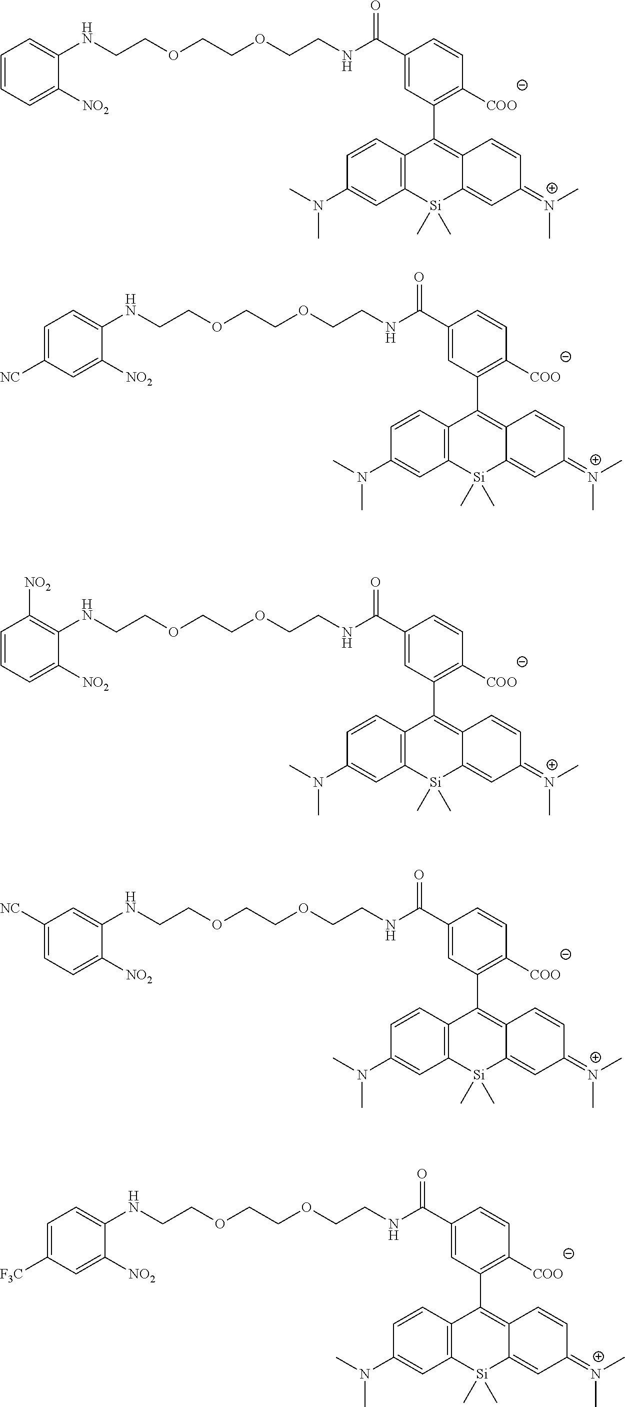

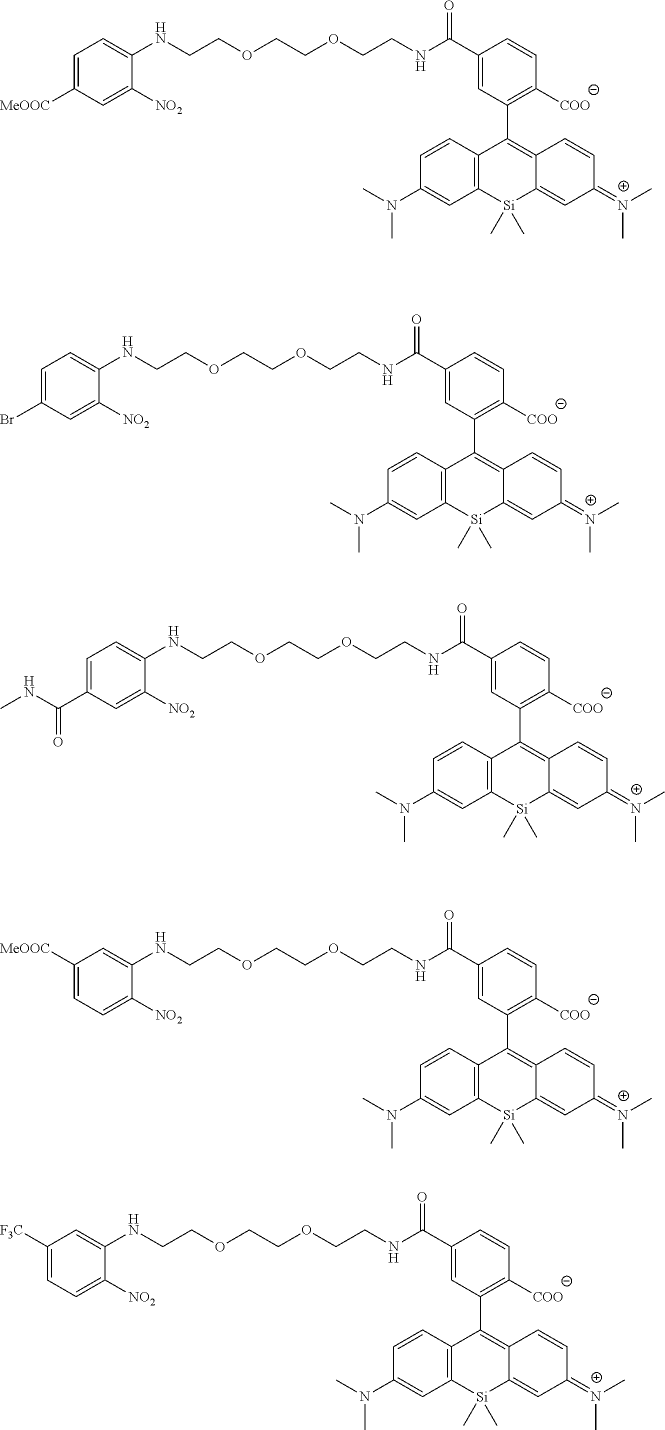

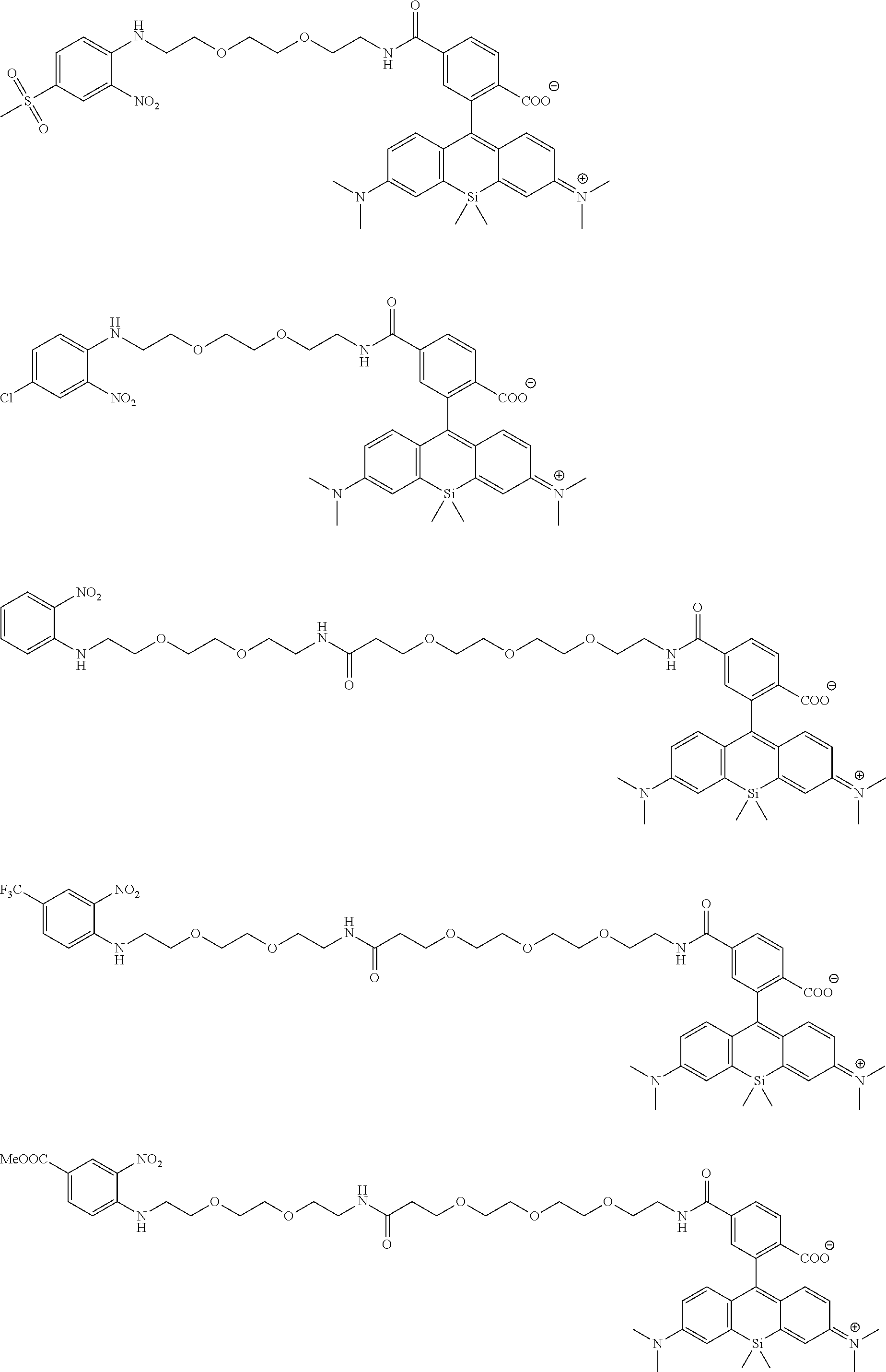

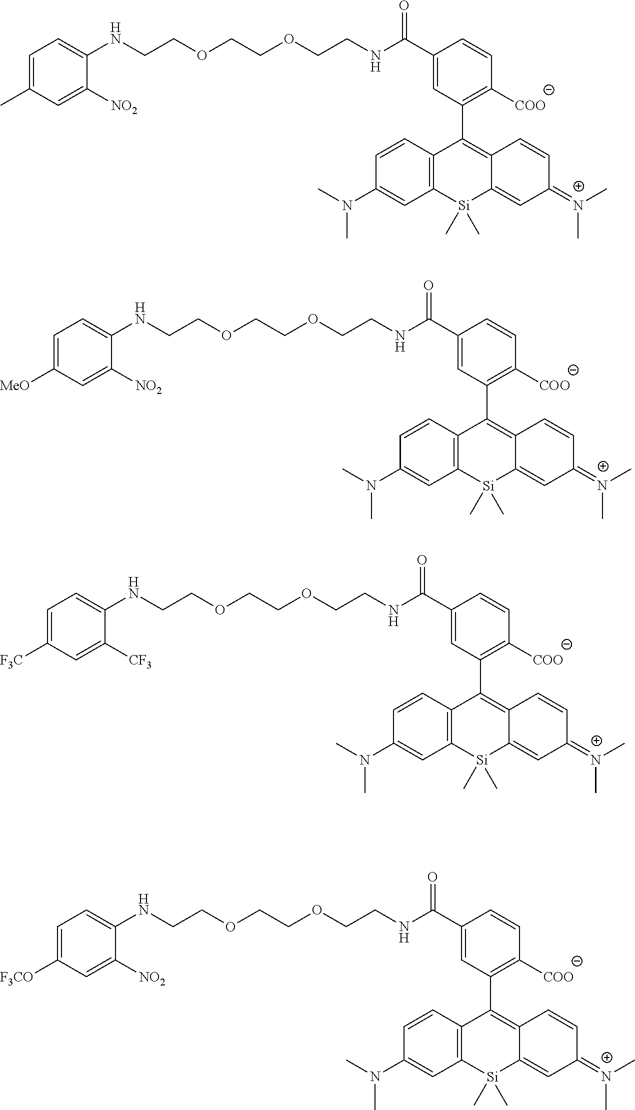

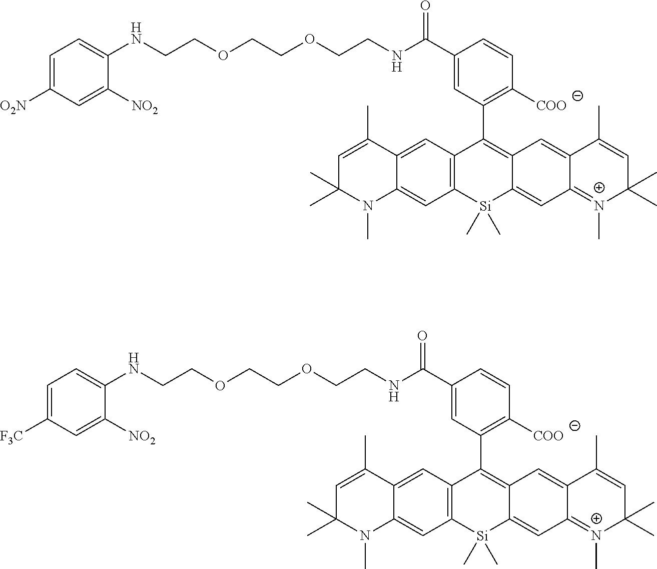

[28] A super-resolution imaging method comprising:

obtaining, in a cell, a fusion protein of a labeling object protein and an anti-DNP (dinitrophenyl compound) antibody;

bringing a compound represented by formula (I) below or a salt thereof into contact with the cell; and

fluorescently labeling the object protein by reacting the fusion protein and the compound represented by formula (I) below or a salt thereof.

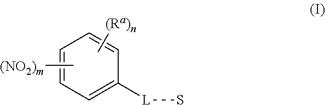

(In the formula (I):

S is a fluorescent group,

L is a linker, and

R^{a} is a monovalent substituent;

m is an integer of 0 to 2, and

n is an integer of 0 to 2;

when m is 2, n is 0;

when m is 1, n is 1 or 0;

when m is 0, n is 2; and

when n is 2, the monovalent substituents of R^{a} may be the same or different.)

[29] The super-resolution imaging method according to [28], using single-molecule localization microscopy.

[30] The super-resolution imaging method according to [28] or [29], wherein the anti-DNP antibody in the fusion protein comprises an amino acid sequence having at least 90% homology to the amino acids of SEQ ID NO: 7 and includes the amino acid sequences represented by SEQ ID NO: 1 to 6, and comprises an amino acid sequence in which at least one of substitutions below is made in the amino acid sequence represented by any of SEQ ID NO: 1 to 6:

(a) any one amino acid from among glutamic acid at position 33, tyrosine at position 37, valine at position 94, glutamine at position 95, glycine at position 159, phenylalanine at position 160, phenylalanine at position 162, asparagine at position 164, glycine at position 233, tyrosine at position 235, tyrosine at position 236, aspartic acid at position 237, arginine at position 239, tyrosine at position 240, and tyrosine at position 242 numbered from an N-terminus is substituted with alanine; or

(b) any one amino acid from among tyrosine at position 96 and tyrosine at position 234 numbered from the N-terminus is substituted with phenylalanine.

[31] The super-resolution imaging method according to any one of [28] to [30], wherein the anti-DNP antibody in the fusion protein comprises an amino acid sequence in which a substitution below is made in the amino acids of SEQ ID NO: 7:

(1) any one amino acid from among glutamic acid at position 33, tyrosine at position 37, valine at position 94, glutamine at position 95, glycine at position 159, phenylalanine at position 160, phenylalanine at position 162, asparagine at position 164, glycine at position 233, tyrosine at position 235, tyrosine at position 236, aspartic acid at position 237, arginine at position 239, tyrosine at position 240, and tyrosine at position 242 numbered from the N-terminus is substituted with alanine; or

(2) any one amino acid from among tyrosine at position 96 and tyrosine at position 234 numbered from the N-terminus is substituted with phenylalanine.

[32] A fluorescent probe used in the super-resolution imaging method according to any one of [28] to [31], the fluorescent probe comprising a compound represented by formula (I) below or a salt thereof.

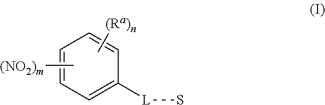

(In the formula (I): S is a fluorescent group, L is a linker, and R^{a} is a monovalent substituent; m is an integer of 0 to 2, n is an integer of 0 to 2; when m is 2, n is 0; when m is 1, n is 1 or 0; when m is 0, n is 2; and when n is 2, the monovalent substituents of R^{a} may be the same or different.)

[33] The fluorescent probe according to [31], wherein the monovalent substituent represented by R^{a} is selected from the group consisting of a halogen atom, a C1-10 alkyl group, a C1-10 alkoxy group, a cyano group, an ester group, an amide group, an alkyl sulfonyl group, a C1-10 alkyl group in which at least one hydrogen atom is substituted with a fluorine atom, and a C1-10 alkoxy group in which at least one hydrogen atom is substituted with a fluorine atom.

[34] The fluorescent probe used in a super-resolution imaging method according to claim 32 or 33, including a compound represented by formula (Ib) below or a salt thereof.

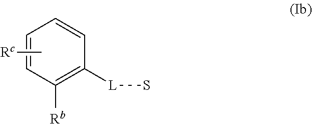

In formula (Ib), S is a fluorescent group, L is a linker, and R^{b} and R^{c} are selected from combinations below. (R^{b}, R^{c}): (NO₂, p-NO₂), (NO₂, p-Br), (NO₂, p-SO₂Me), (NO₂, p-Cl), (NO₂, m-CN), (NO₂, p-CN), (NO₂, p-COOMe), (CF₃, p-CF₃), (NO₂, p-CONHMe), (NO₂, m-COOMe), (NO₂, H)

(Here, p- and m- represent R^{c} being in a para position and a meta position on the benzene ring, respectively, with respect to L.)

### Advantages of the Invention

Through the present invention, it is possible to provide a novel and useful molecular tagging technique provided with a function for controlling an ON/OFF state of fluorescence in a cell.

Through use of the molecular tagging technique of the present invention, an excellent method for fluorescently labeling an intracellular protein can be provided, and by the fluorescent labeling method of the present invention, it is possible to observe, by fluorescence, localization of a fluorescent molecule in a living cell with high contrast under a condition of extremely low background fluorescence. Furthermore, structured illumination microscopy (SIM) in live-cell imaging or super-resolution imaging of a functional molecule labeled in a living cell is made possible by the fluorescence labeling method of the present invention.

It is expected that by applying the molecular tagging technique of the present invention to live-cell imaging or super-resolution imaging, tracking of molecular movement or analysis of fine structures at a nanoscale spatial resolution will be realized, and a contribution will be made to elucidating molecular mechanisms on which cellular function is based.

Through the present invention, highly practical super-resolution imaging can be realized by controlling binding/dissociation kinetics of a fluorescent probe in which fluorescence is OFF (QODE probe) and a molecular tag (De-QODE tag) in which quenching is removed and fluorescence is turned ON by binding of the molecular tag with an anti-quencher antibody.

### BRIEF DESCRIPTION OF THE DRAWINGS

FIG. 1 Design of a molecular tagging technique for enabling ON/OFF control of fluorescence according to the present invention.

FIG. 2 Development scheme for the molecular tagging technique using a quenching phenomenon according to an example.

FIG. 3 ELISA and fluorescence screening of anti-DNP monoclonal antibodies (A: Results of anti-DNP monoclonal antibody screening by ELISA. B: Results of anti-DNP monoclonal antibody screening indexed to increase in fluorescence intensity of hybridoma supernatant by SRB-DNP. C: Fluorescence change rate for four types of fluorophore-DNP pairs in a hybridoma culture supernatant in 27 wells having the highest SRB-DNP fluorescence change rate.)

FIG. 4 Amino acid sequence of an anti-DNP scFv.

FIG. 5 Results of anti-DNP scFv expression tests in cultured cells.

FIG. 6 Absorption and fluorescence spectra of 6SiR-DNP (A: Structural formula of 6SiR-DNP. B: Absorption spectrum of 6SiR-DNP. C: Fluorescence spectrum of 6SiR-DNP. Dashed lines indicate the fluorescence spectra in the absence of 5D4, and solid lines indicate the absorption spectra in the presence of 2.5 μM 5D4.)

FIG. 7 Absorption spectra of 60G-DNP, 6DCF-DNP, 6JF549-DNP, 6SiR600-DNP, 6SiR-DNP, and 6SiR700-DNP (shown in the order 6OG-DNP, 6DCF-DNP, 6JF549-DNP, 6SiR600-DNP, 6SiR-DNP, 6SiR700-DNP from a short-wavelength side).

FIG. 8 Fluorescence images of a cell expressing a molecular tag to which an organelle-localized peptide is added. Shows differential interference contrast (DIC) microscope images and ECFP and 6SiR-DNP fluorescence images. (A: Fluorescence images of a cell in which only ECFP is expressed in cytoplasm. B: Fluorescence images when ECFP-5D4 is expressed in the cytoplasm but 6SiR-DNP is not loaded. C: Fluorescence images of a cell in which ECFP-5D4 is expressed in the cytoplasm. D, E, F: DIC image and ECF and 6SiR-DNP fluorescence images of a HeLa cell in which ECFP-5D4 having a nucleus (D), cell membrane (E), and endoplasmic reticulum (F) localization signal sequence, respectively, added thereto is expressed. Images below F are enlargements of the area in the yellow frame. Scale bars represent 10 μm in full cell images and 2 μm only in the enlarged images.)

FIG. 9 Fluorescence images of a cell in which a fusion protein of an intracellular molecule and a molecular tag is expressed. (A: Fluorescence images of 6SiR-DNP in a HeLa cell expressing a fusion protein of tubulin and 5D4. B: Fluorescence images of 6SiR-DNP in a HeLa cell expressing a fusion protein of actin and 5D4. C: Fluorescence images of 6SiR-DNP in a HeLa cell expressing a fusion protein of actin-binding peptide LifeAct and 5D4. D: Fluorescence images of 6SiR-DNP in a HeLa cell expressing a fusion protein of STIM1 and 5D4. Scale bars represent 10 μm in full cell images and 2 μm in enlarged images.)

FIG. 10 Results of live-cell imaging of STIM1-5D4. Time-lapse imaging images of a HeLa cell expressing STIM1-5D4. (An enlarged view of the yellow frame is shown below each frame. Arrowheads indicate a molecule of interest. Scale bars represent 10 μm in full cell images and 2 μm in enlarged images.)

FIG. 11 Results of super-resolution imaging by SIM of a living cell specimen. (A: Normal fluorescence image of a HeLa cell expressing tubulin-5D4. B: SIM image of a HeLa cell expressing tubulin-5D4. C: Time-lapse SIM images of a HeLa cell expressing tubulin-5D4. Enlarged views of portions enclosed by white frames in D and C show two fields of view. Arrowheads indicate microstructures of interest. Scale bars represent 2 μm in A, B, and C, and 500 nm in D.)

FIG. 12 Schematic views illustrating binding/dissociation kinetics of 6DCF-DNP and 5D4.

FIG. 13 Results of 5D4 point-mutagenesis screening.

FIG. 14 Super-resolution imaging of endoplasmic reticulum in a living cell.

FIG. 15 Specific in vivo imaging of cells expressing 5D4.

FIG. 16 Long-term-stable fluorescence imaging based on tag/probe binding/dissociation equilibrium.

### BEST MODE FOR CARRYING OUT THE INVENTION

One embodiment of the present invention is a method for fluorescently labeling an intracellular protein, the method for fluorescently labeling a protein comprising obtaining, in a cell, a fusion protein of a labeling object protein and an anti-DNP (dinitrophenyl compound) antibody, bringing a compound represented by formula (I) or a salt thereof into contact with the cell, and fluorescently labeling the object protein by reacting the fusion protein and the compound represented by formula (I) or a salt thereof.

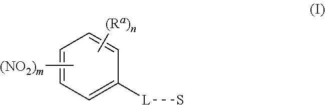

In formula (I): S is a fluorescent group, L is a linker, and R^{a} is a monovalent substituent; m is an integer of 0 to 2, and n is an integer of 0 to 2; when m is 2, n is 0; when m is 1, n is 1 or 0; when m is 0, n is 2; and when n is 2, the monovalent substituents of R^{a} may be the same or different.

Another embodiment of the present invention is a method for fluorescently labeling an intracellular protein, the method for fluorescently labeling a protein including obtaining, in a cell, a fusion protein of a labeling object protein and an anti-DNP (dinitrophenyl compound) antibody, bringing a compound represented by formula (Ia) or a salt thereof into contact with the cell, and fluorescently labeling the object protein by reacting the fusion protein and the compound represented by formula (Ia) or a salt thereof.

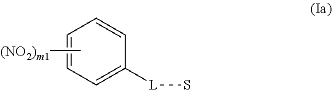

In formula (1a), S is a fluorescent group, L is a linker, and m1 is 1 or 2.

Specifically, of importance in the present invention is ON/OFF control of fluorescence through use of a chemical mechanism using a specific anti-DNP antibody, in which quenching is removed when the antibody is bound to a fluorophore-dye pair.

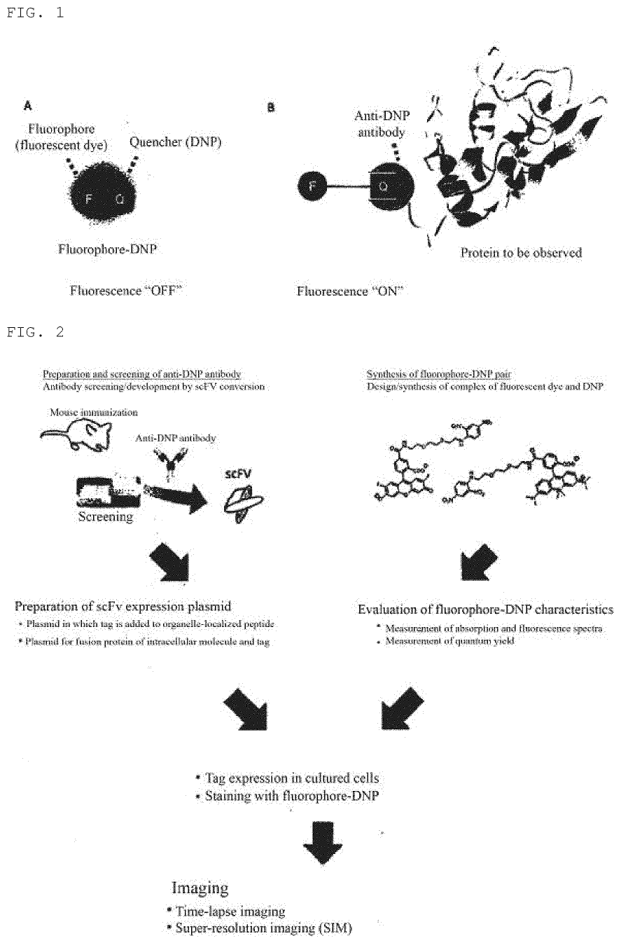

A conceptual diagram of a molecular tagging technique which makes the ON/OFF control of the present invention possible is shown in FIG. 1. In the diagram, F represents a fluorescent dye, and Q represents a quencher. When the fluorescent dye and DNP (dinitrophenyl group) are in proximity, fluorescence is not emitted, being quenched by DNP in a steady state (A in FIG. 1; schematic view when fluorescence is OFF). Meanwhile, when DNP and the anti-DNP antibody expressed in the cell bind, the quenching ability of DNP is eliminated, and the fluorophore-dye pair becomes fluorescent (B in FIG. 1; schematic view when fluorescence is ON).

The fluorescent labeling method of the present invention includes obtaining, in a cell, a fusion protein of a labeling object protein and an anti-DNP antibody.

The anti-DNP antibody in the fusion protein obtained in a cell in the method of the present invention (also referred to hereinbelow as the “anti-DNP antibody of the present invention”) is an antibody or an antigen-binding fragment in which only variable regions of heavy and light chains of the antibody are connected by a short amino acid linker.

The anti-DNP antibody of the present invention is preferably a single-chain Fv (scFv). The anti-DNP antibody of the present invention is also preferably an antibody having a molecular weight of about 30 kDa.

An antibody is ordinarily a molecule having a molecular weight of about 60 kDa in which heavy chains and light chains are connected by disulfide bonds. Due to a reductive environment inside the cell, a full-length antibody is not suitable for formation of the plurality of disulfide bonds that are necessary for normal folding, and it is difficult to express a full-length antibody in a cell while maintaining a normal folding state. In contrast, the antibody of the present invention has a structure in which only the variable regions of the heavy and light chains of the antibody are connected by a short amino acid linker, and the antibody of the present invention is therefore relatively easy to express in a cell.

In a preferred embodiment of the present invention, the anti-DNP antibody is an anti-DNP antibody or an antigen-binding fragment thereof comprising a light chain including a VL-CDR1 comprising the amino acid sequence represented by SEQ ID NO: 1, a VL-CDR2 comprising the amino acid sequence represented by SEQ ID NO: 2, and a VL-CDR3 comprising the amino acid sequence represented by SEQ ID NO: 3, and a heavy chain including a VH-CDR1 comprising the amino acid sequence represented by SEQ ID NO: 4, a VH-CDR2 comprising the amino acid sequence represented by SEQ ID NO: 5, and a VH-CDR3 comprising the amino acid sequence represented by SEQ ID NO: 6.

Sequence No. 1:
QEISGY
Sequence No. 2:
AAS
Sequence No. 3:
VQYASYPYT
Sequence No. 4:
GFTFSNYWMNW
Sequence No. 5:
IRLKSNNYAT
Sequence No. 6:
TGYYYDSRYGY

The abovementioned anti-DNP antibody or antigen-binding fragment thereof is preferably a single-chain Fv (scFV).

Anti-DNP antibodies have been widely used in immunological research, and are known as haptens (incomplete antigens). However, in order to control fluorescence of a dye compound through control of quenching ability by an anti-DNP antibody, the anti-DNP antibody must be stably expressed in a cell. Therefore, in the anti-DNP antibody used in the method of the present invention, efficiency of intracellular expression thereof can be enhanced by reducing a size of the antibody and configuring the antibody as a single-chain antibody (scFv).

According to a preferred aspect of the anti-DNP antibody of the present invention, the anti-DNP antibody is an antibody or antigen-binding fragment thereof comprising an amino acid sequence having at least 90%, preferably at least 95%, and more preferably at least 98% homology to the amino acids of SEQ ID NO: 7 below and including: a light chain including a VL-CDR1 comprising the amino acid sequence represented by SEQ ID NO: 1, a VL-CDR2 comprising the amino acid sequence represented by SEQ ID NO: 2, and a VL-CDR3 comprising the amino acid sequence represented by SEQ ID NO: 3; and a VH-CDR1 comprising the amino acid sequence represented by SEQ ID NO: 4, a VH-CDR2 comprising the amino acid sequence represented by SEQ ID NO: 5, and a VH-CDR3 comprising the amino acid sequence represented by SEQ ID NO: 6.

Sequence No. 7:
MADYKDIVLTQSPSSLSASLGERVSLTCRSSQEISGYLGWLQQKPDGSIK
RLIYAASTLDSGVPKRFSGSRSGSDYSLTISSLESEDFADYYCVQYASYP
YTFGGGTKLEMKRGGGGSGGGGSGGGGSGGGGSQIQLQESGGGLVQPGGS
MKLSCVASGFTFSNYWMNWVRQSPEKGLDWVAEIRLKSNNYATHYAESVK
GRFTISRDDSKSSVYLQMNNLRAEDTGIYYCTGYYYDSRYGYWGQGTTVT
VSS

According to a preferred aspect of the anti-DNP antibody of the present invention, the anti-DNP antibody is an antibody or antigen-binding fragment thereof having the amino acid sequence represented by SEQ ID NO: 7.

In the fluorescent labeling method of the present invention, use of an scFv anti-DNP antibody derived from a mouse or the like such as described above is preferred.

Besides an scFv antibody derived from a mouse or the like, a heavy-chain antibody (VHH) produced by a llama or another animal of the family Camelidae may be used in the fluorescent labeling method of the present invention. A VHH is a single-domain antibody which is constituted solely from a heavy chain and has a molecular weight of approximately 15 kDa, and can therefore readily fuse with another protein or peptide or be expressed intracellularly. A CDR3 region thereof is also longer than that of another IgG antibody, and a VHH can therefore readily have high affinity for an antigen, and because a VHH has the property of readily winding back to a natural structure thereof even when modified, a VHH is extremely useful as a tag.

In the fluorescent labeling method of the present invention, obtaining of the fusion protein of the labeling object protein and the anti-DNP antibody may include obtaining a polynucleotide coding for the fusion protein, obtaining a plasmid or vector capable of expressing the fusion protein, causing the fusion protein to be expressed in a cell, or isolating the expressed fusion protein.

A plasmid or vector capable of expressing the fusion protein can be prepared in accordance with a usual method using a polynucleotide coding for the labeling object protein, a polynucleotide coding for the anti-DNP antibody, etc., as polynucleotides coding for the fusion protein.

The fusion protein can generally be prepared using a standard technique (including chemical conjugation). In brief, DNA sequences coding for polypeptide components can be separately assembled, and can be connected as an appropriate expression vector. A 3′-end of the DNA sequence coding for one polypeptide component is connected to a 5′-end of the DNA sequence coding for a second polypeptide component with or without the use of a peptide linker, and as a result, reading frames of the sequences are placed in phase (the phases thereof are matched). It is thereby possible for a single fusion peptide to be translated which retains the biological activity of both of the component peptides.

A linker sequence can be used to separate the first polypeptide and the second polypeptide at an adequate distance from each other, and each polypeptide can be expected to fold into a higher-order structure thereof and to not inhibit a function of the other. The linker may be a peptide, a polypeptide, an alkyl chain, or another conventional-type spacer molecule.

Any protein can be used as the labeling object protein, examples thereof including cytoskeletal proteins, ion channels, and receptors.

In the fluorescent labeling method of the present invention, the fusion protein is preferably obtained by introducing the plasmid or vector capable of expressing the fusion protein into a cell or an organism.

The fluorescent labeling method of the present invention includes a cell in which the abovementioned fusion protein is obtained, and bringing a compound represented by formula (I) or a salt thereof into contact with the cell.

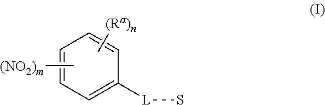

In formula (I): S is a fluorescent group, L is a linker, and R^{a} is a monovalent substituent.

Also in formula (I): m is an integer of 0 to 2, and n is an integer of 0 to 2; when m is 2, n is 0; when m is 1, n is 1 or 0; when m is 0, n is 2; and when n is 2, the monovalent substituents of R^{a} may be the same or different.

In the present specification, an “alkyl group” or an alkyl component of a substituent (e.g., an alkoxy group or the like) including an alkyl component means an alkyl group comprising, e.g., a C1-6, preferably a C1-4, and more preferably a C1-3 straight-chain, branched-chain, or cyclic alkyl group or a combination thereof, unless otherwise specified. More specifically, an alkyl group may be, for example, a methyl group, an ethyl group, an n-propyl group, an isopropyl group, a cyclopropyl group, an n-butyl group, a sec-butyl group, an isobutyl group, a tert-butyl group, a cyclopropylmethyl group, an n-pentyl group, an n-hexyl group, or the like.

A “halogen atom” in the present specification may be a fluorine atom, a chlorine atom, a bromine atom, or an iodine atom, and is preferably a fluorine atom, a chlorine atom, or a bromine atom.

The monovalent substituent represented by R^{a} is selected from the group consisting of a halogen atom, a C1-10 alkyl group, a C1-10 alkoxy group, a cyano group, an ester group, an amide group, an alkyl sulfonyl group, a C1-10 alkyl group in which at least one hydrogen atom is substituted with a fluorine atom, and a C1-10 alkoxy group in which at least one hydrogen atom is substituted with a fluorine atom.

When the monovalent substituent of R^{a} is an alkyl group, the alkyl group is preferably a methyl group.

The alkoxy group is preferably a methoxy group.

The ester group is preferably a methyl ester group.

The amide group is preferably a methyl amide group.

The alkyl sulfonyl group is preferably a methylsulfonyl group.

The alkyl group in which at least one hydrogen atom is substituted with a fluorine atom is preferably a trifluoromethyl group.

The alkoxy group in which at least one hydrogen atom is substituted with a fluorine atom is preferably a trifluoromethoxy group.

In formula (I), m is an integer of 0 to 2, and n is an integer of 0 to 2.

When m is 2, n is 0; i.e., the compound of formula (I) has a structure in which two nitro groups are bonded to a benzene ring.

When m is 1, n is 1 or 0. Here, when n is 1, the compound of formula (I) has a structure in which a single R^{a} is bonded to a single nitro group, and when n is 0, the compound of formula (I) has a structure in which a single nitro group is bonded to a benzene ring.

When m is 0, n is 2; i.e., the compound of formula (I) has a structure in which two R^{a} groups are bonded to a benzene ring.

A compound in which m is 2 and n is 0, and a compound in which m is 1 and n is 1 in formula (I) are also represented by formula (Ia) below.

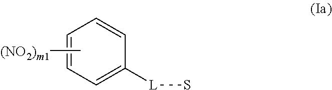

In formula (Ia), S is a fluorescent group, L is a linker, and m1 is 1 or 2.

In the description below, the compound represented by formula (I) and the salt thereof and the compound represented by formula (Ia) and the salt thereof are also referred to collectively as the “compound of the present invention.”

Specifically, the fluorescent labeling method of the present invention includes a cell in which the abovementioned fusion protein is obtained, and bringing a compound represented by formula (Ia) or a salt thereof into contact with the cell.

The linker in formulas (I) and (Ia) can be represented by T-Y, where Y is a bonding group for bonding with the fluorescent group S, and T represents a crosslinking group.

The bonding group represented by Y is selected from an amide group (—CONH—, —CONR′—, —R—CONH—, or —R—CONR′—), an alkylamide group (—CONH—R— or —CONR′—R—), an ester group (—COO—), an alkylester group (—R—COO— or —COO—R—), a carbonylamino group (—NHCO— or —NR′CO—), or an alkylether group (—RO— or —OR—). In these groups, R represents a divalent hydrocarbon group, preferably a C1-10 alkylene group, and more preferably a C1-5 alkylene group, and R′ represents a C1-5 alkyl.

Any crosslinking group which works as a spacer for connecting the bonding group Y and the benzene ring of the compound of formula (I) or (Ia) can be used as the crosslinking group T. Examples thereof include, but are not limited to, substituted or unsubstituted divalent hydrocarbon groups (alkanes, alkenes, alkynes, cycloalkanes, aromatic hydrocarbons, and the like), dialkylether groups (e.g., dimethyl ether, diethyl ether, methylethyl ether, and the like), an ethylene glycol group, a diethylene glycol group, a triethylene glycol group, a polyethylene glycol group, an amide group, a carbonyl or the like, and heterocyclic groups (e.g., a divalent piperidine ring or the like), and combinations of two or more of the above groups. The crosslinking group may have, at one or both ends thereof, a functional group capable of bonding to Y and the benzene ring of the compound of formula (I) or (Ia), examples of such a functional group including an amino group, an alkylamino group, an aminoalkyl group, a carbonyl group, a carboxyl group, an amide group, an alkylamide group, and the like.

The crosslinking group T also includes a group represented by the formula T₁-(W)-T₂. Each of the crosslinking groups presented as examples above can be used as T₁ and T₂. The group W, when present, is a group for connecting T: and T₂, and examples thereof include an amino group, an alkylamino group, an aminoalkyl group, a carbonyl group, a carboxyl group, an amide group, an alkylamide group, and the like.

Examples of such a crosslinking group include, but are not limited to, a group in which a triethylene glycol group and a diethylene glycol group are bonded via an amide group, an alkylamide group, or the like. Furthermore, the crosslinking group represented by the formula T₁-(W)-T₂ may have, at one or both ends thereof, a functional group (e.g., an amino group, an alkylamino group, an aminoalkyl group, a carbonyl group, a carboxyl group, an amide group, an alkylamide group, or the like) capable of bonding to Y and the benzene ring of the compound of formula (I) or (Ia).

In formula (Ia), m1 is 1 or 2, but is preferably 2.

In the compound of formula (Ia), when m1 is 1, the nitro group is preferably in an ortho position or a para position on the benzene ring with respect to L, and when m1 is 2, a nitro group is preferably in the ortho position and the para position on the benzene ring with respect to L.

The group S is a fluorescent dye, and is preferably a xanthene dye, a cyanine dye, a coumarin dye, a dipyrromethene dye, or a benzophenoxazine dye.

According to a preferred aspect of the compound of the present invention, S is represented by formula (II) below.

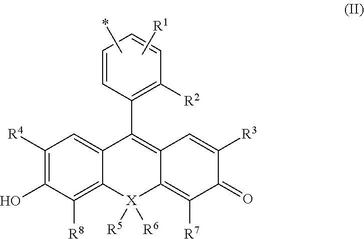

In formula (II), R¹ represents a hydrogen atom or one to four same or different monovalent substituents which are present on a benzene ring.

A type of the monovalent substituent represented by R¹ is not particularly limited, but is preferably selected from the group consisting of a C1-6 alkyl group, a C1-6 alkenyl group, a C1-6 alkynyl group, a C1-6 alkoxy group, a hydroxyl group, a carboxy group, a sulfonyl group, an alkoxycarbonyl group, a halogen atom, and an amino group, for example. These monovalent substituents may have any one or more substituents. For example, one or more halogen atoms, carboxy groups, sulfonyl groups, hydroxyl groups, amino groups, alkoxy groups, or the like may be present on the alkyl group represented by R¹, and the alkyl group represented by R¹ may be a halogenated alkyl group, a hydroxyalkyl group, a carboxyalkyl group, or an aminoalkyl group or the like, for example.

In a preferred embodiment of the present invention, R¹ are all hydrogen atoms.

In formula (II), R² represents a hydrogen atom, a monovalent substituent, or a bond. A type of the monovalent substituent represented by R² is not particularly limited, but as in the case of R¹, R² is a C1-6 alkyl group, a C1-6 alkenyl group, a C1-6 alkynyl group, a C1-6 alkoxy group, a hydroxyl group, a carboxy group, a sulfonyl group, an alkoxycarbonyl group, a halogen atom, an amino group, or the like, for example.

In a preferred embodiment of the present invention, R² is a C1-6 alkyl group (preferably a methyl group), a carboxyl group, a methoxy group, a hydroxymethyl group, or a bond (specifically, L (i.e., the linker) is introduced at the position of R²).

In formula (II), R³ and R⁴ each independently represent a hydrogen atom, a C1-6 alkyl group, or a halogen atom.

When R³ or R⁴ represents an alkyl group, one or more of a halogen atom, a carboxy group, a sulfonyl group, a hydroxyl group, an amino group, an alkoxy group, or the like may be present in the alkyl group; for example, the alkyl group represented by R³ or R⁴ may be a halogenated alkyl group, a hydroxyalkyl group, a carboxyalkyl group, or the like. R³ and R⁴ are preferably each independently a hydrogen atom or a halogen atom, and a case in which both R³ and R⁴ are hydrogen atoms or a case in which both R³ and R⁴ are fluorine atoms or chlorine atoms is more preferred.

In formula (II), R⁵ and R⁶ each independently represent a C1-6 alkyl group, an aryl group, or a bond, provided that R⁵ and R⁶ being absent when X is an oxygen atom.

When X is a silicon atom, R⁵ and R⁶ are preferably each independently a C1-3 alkyl group, and more preferably, both R⁵ and R⁶ are methyl groups. One or more of a halogen atom, a carboxy group, a sulfonyl group, a hydroxyl group, an amino group, an alkoxy group, or the like may be present in the alkyl groups represented by R⁵ and R⁶; for example, the alkyl group represented by R⁵ or R⁶ may be a halogenated alkyl group, a hydroxyalkyl group, a carboxyalkyl group, or the like. When R⁵ or R⁶ represents an aryl group, the aryl group may be a monocyclic aromatic group or a condensed aromatic group, and an aryl ring may include one or more ring-forming hetero atoms (e.g., nitrogen atoms, oxygen atoms, sulfur atoms, and the like). The aryl group is preferably a phenyl group. One or more substituents may be present on the aryl ring. One or more halogen atoms, carboxy groups, sulfonyl groups, hydroxyl groups, amino groups, alkoxy groups, or the like, for example, may be present as the substituent.

In formula (II), R⁷ and R⁸ each independently represent a hydrogen atom, a C1-6 alkyl group, a halogen atom, or a bond, and are the same as described above with regard to R³ and R⁴. Preferably, both R⁷ and R⁸ are hydrogen atoms, chlorine atoms, or fluorine atoms.

X represents an oxygen atom or a silicon atom. Preferably, X is an oxygen atom.

The symbol * represents a location of bonding (bonding point; the same hereinbelow) with L in formula (I) or formula (Ia) at any position on the benzene ring. Preferably, L can bond at any position of the benzene ring bonded to the xanthene ring skeleton, but L is preferably bonded at position 4 of the benzene ring.

According to a preferred aspect of the present invention, S is represented by formula (III) below.

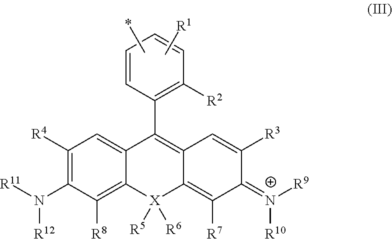

In formula (III), R: through R⁸ and X are as described above with regard to formula (II).

In formula (III), R⁹ and R¹⁰ each independently represent a hydrogen atom or a C1-6 alkyl group.

The groups R⁹ and R¹⁰ may also together form a 4- to 7-membered heterocyclyl which includes a nitrogen atom to which R⁹ and R¹⁰ are bonded.

Either R⁹ or R¹⁰, or both R⁹ and R¹⁰ may also respectively combine with R³ or R⁷ to form a 5- to 7-membered heterocyclyl or heteroaryl which includes a nitrogen atom to which R⁹ or R⁰ is bonded. One to three additional hetero atoms selected from the group consisting of an oxygen atom, a nitrogen atom, and a sulfur atom may be contained as ring-forming members, and the heterocyclyl or heteroaryl may be furthermore substituted with a C1-6 alkyl, a C2-6 alkenyl, or a C2-6 alkynyl, a C6-10 aralkyl group, or a C6-10 alkyl-substituted alkenyl group. In this case, the heterocyclyl or heteroaryl can have one or more substituents.

In formula (III), R¹¹ and R¹² each independently represent a hydrogen atom or a C1-3 alkyl group.

The groups R¹¹ and R¹² may also together form a 4- to 7-membered heterocyclyl which includes a nitrogen atom to which R¹¹ and R¹² are bonded.

Either R¹¹ or R¹², or both R¹¹ and R¹² may also respectively combine with R⁴ or R⁸ to form a 5- to 7-membered heterocyclyl or heteroaryl which includes a nitrogen atom to which R¹¹ or R¹² is bonded. One to three additional hetero atoms selected from the group consisting of an oxygen atom, a nitrogen atom, and a sulfur atom may be contained as ring-forming members, and the heterocyclyl or heteroaryl may be furthermore substituted with a C1-6 alkyl, a C2-6 alkenyl, or a C2-6 alkynyl, a C6-10 aralkyl group, or a C6-10 alkyl-substituted alkenyl group. In this case, the heterocyclyl or heteroaryl can have one or more substituents.

In formula (III), the symbol * represents a location of bonding (bonding point; the same hereinbelow) with L in formula (I) or formula (Ia) at any position on the benzene ring. Preferably, L can bond at any position of the benzene ring bonded to the xanthene ring skeleton, but L is preferably bonded at position 4 of the benzene ring.

The fluorescent labeling method of the present invention includes fluorescently labeling the object protein by reacting the fusion protein and the compound represented by formula (I) or a salt thereof.

In the fluorescent labeling method of the present invention, the step for reacting the fusion protein and the compound represented by formula (I) or a salt thereof may be performed in an organism or in a cell in which the fusion protein is expressed, or may be performed in vitro using the isolated fusion protein. When labeling is performed in vitro, labeling may be performed in a buffer solution (pH 7.4) at a temperature of 25° C., for example.

In a steady state, fluorescence of the compound of the present invention is quenched by DNP and is not emitted (see A in FIG. 1), but when DNP and the anti-DNP antibody expressed in the cell bind, the quenching ability of DNP is eliminated, the fluorophore-dye pair becomes fluorescent, and the labeling object protein in the cell can be fluorescently labeled.

The compound of the present invention is fully quenched when not bound to the anti-DNP antibody, and even when the compound of the present invention is present outside the cell, the effect of fluorescence originating from the compound of the present invention not bound to the anti-DNP antibody on spatial resolution in observation of organelles or molecules being observed is suppressed to a negligible level. In the fluorescent labeling method of the present invention, there is no need for a step for removing an unnecessary fluorescent dye from a system during fluorescence observation, and this feature is particularly useful in high-throughput screening (HTS) for drug discovery and the like. In HTS, efficiency of a screening system as a whole is increased by reducing the number of steps such as probe washing, and numerous specimens are required to be assayed at extremely high efficiency. A method in which washing and other processing is omitted and reaction and measurement are performed successively is referred to as a “mix and measure” or “homogeneous” method, and such a method is considered desirable particularly in drug screening in which tens of thousands to hundreds of thousands of compounds are assayed. In a screening system in which a DNP tag of the present invention and the fluorophore-dye pair are introduced, an HTS system can be constructed in which there is no need for a washing process for excess fluorescent dye.

Another embodiment of the present invention is an anti-DNP antibody or an antigen-binding fragment thereof (also referred to below as the “anti-DNP antibody 1 or antigen-binding fragment 1 thereof”) comprising a light chain including a VL-CDR1 comprising the amino acid sequence represented by SEQ ID NO: 1, a VL-CDR2 comprising the amino acid sequence represented by SEQ ID NO: 2, and a VL-CDR3 comprising the amino acid sequence represented by SEQ ID NO: 3, and a heavy chain including a VH-CDR1 comprising the amino acid sequence represented by SEQ ID NO: 4, a VH-CDR2 comprising the amino acid sequence represented by SEQ ID NO: 5, and a VH-CDR3 comprising the amino acid sequence represented by SEQ ID NO: 6.

Sequence No. 1:
QEISGY
Sequence No. 2:
AAS
Sequence No. 3:
VQYASYPYT
Sequence No. 4:
GFTFSNYWMNW
Sequence No. 5:
IRLKSNNYAT
Sequence NO. 6:
TGYYYDSRYGY

The anti-DNP antibody or antigen-binding fragment thereof of the present invention is preferably a single-chain Fv (scFv).

According to a preferred aspect of the anti-DNP antibody of the present invention, the anti-DNP antibody is an antibody or antigen-binding fragment thereof (also referred to below as the “anti-DNP antibody 2 or antigen-binding fragment 2 thereof”) comprising an amino acid sequence having at least 90%, preferably at least 95%, and more preferably at least 98% homology to the amino acids of SEQ ID NO: 7 below and including: a light chain including a VL-CDR1 comprising the amino acid sequence represented by SEQ ID NO: 1, a VL-CDR2 comprising the amino acid sequence represented by SEQ ID NO: 2, and a VL-CDR3 comprising the amino acid sequence represented by SEQ ID NO: 3; and a VH-CDR1 comprising the amino acid sequence represented by SEQ ID NO: 4, a VH-CDR2 comprising the amino acid sequence represented by SEQ ID NO: 5, and a VH-CDR3 comprising the amino acid sequence represented by SEQ ID NO: 6.

Sequence No. 7:
MADYKDIVLTQSPSSLSASLGERVSLTCRSSQEISGYLGWLQQKPDGSIK
RLIYAASTLDSGVPKRFSGSRSGSDYSLTISSLESEDFADYYCVQYASYP
YTFGGGTKLEMKRGGGGSGGGGSGGGGSGGGGSQIQLQESGGGLVQPGGS
MKLSCVASGFTFSNYWMNWVRQSPEKGLDWVAEIRLKSNNYATHYAESVK
GRFTISRDDSKSSVYLQMNNLRAEDTGIYYCTGYYYDSRYGYWGQGTTVT
VSS

According to a preferred aspect of the anti-DNP antibody of the present invention, the anti-DNP antibody is an antibody or antigen-binding fragment thereof having the amino acid sequence represented by SEQ ID NO: 7 (also referred to below as the “anti-DNP antibody 3″ or antigen-binding fragment 3 thereof”).

Identity and similarity of the anti-DNP antibody can easily be computed by known methods. Such methods include, but are not limited to, the methods described in: Computational Molecular Biology, Lesk, A. M. (Ed.), Oxford University Press, New York (1988); Biocomputing: Informatics and Genome Projects, Smith, D. W. (Ed.), Academic Press, New York (1993); Computer Analysis of Sequence Data, Part 1, Griffin, A. M. and Griffin, H. G. (Eds.), Humana Press, New Jersey (1994); Sequence Analysis in Molecular Biology, von Heinje, G., Academic Press (1987); Sequence Analysis Primer, Gribskov, M. and Devereux, J. (Eds.), M. Stockton Press, New York (1991); and Carillo et al., SIAM J. Applied Math., 48: 1073 (1988).

According to another preferred aspect of the anti-DNP antibody of the present invention, the anti-DNP antibody or antigen-binding fragment thereof (also referred to below as the “anti-DNP antibody 4 or antigen-binding fragment 4 thereof”) comprises an amino acid sequence having at least 90%, preferably at least 95%, and more preferably at least 98% homology to the amino acids of SEQ ID NO: 7 and includes the amino acid sequences represented by SEQ ID NO: 1 through 6, and which comprises an amino acid sequence in which at least one, preferably one, of the substitutions below is made in the amino acid sequence represented by any of SEQ ID NO: 1 through 6:

(a) any one amino acid from among glutamic acid at position 33, tyrosine at position 37, valine at position 94, glutamine at position 95, glycine at position 159, phenylalanine at position 160, phenylalanine at position 162, asparagine at position 164, glycine at position 233, tyrosine at position 235, tyrosine at position 236, aspartic acid at position 237, arginine at position 239, tyrosine at position 240, and tyrosine at position 242 numbered from the N-terminus in SEQ ID NO: 7 is substituted with alanine; or

(b) any one amino acid from among tyrosine at position 96 and tyrosine at position 234 numbered from the N-terminus in SEQ ID NO: 7 is substituted with phenylalanine.

When application of the method of the present invention for fluorescently labeling an intracellular protein to super-resolution imaging, particularly super-resolution imaging using single-molecule localization microscopy, was investigated, it was discovered that binding/dissociation kinetics (k_{off}) of a QODE probe and a molecular tag (De-QODE tag) in which quenching is removed and fluorescence is turned ON by binding with an anti-quencher antibody can be increased by substituting at least one amino acid with alanine or phenylalanine in any of the VL-CDR1 comprising the amino acid sequence represented by SEQ ID NO: 1, the VL-CDR2 comprising the amino acid sequence represented by SEQ ID NO: 2, and the VL-CDR3 comprising the amino acid sequence represented by SEQ ID NO: 3, and the VH-CDR1 comprising the amino acid sequence represented by SEQ ID NO: 4, the VH-CDR2 comprising the amino acid sequence represented by SEQ ID NO: 5, and the VH-CDR3 comprising the amino acid sequence represented by SEQ ID NO: 6.

More specifically, the anti-DNP antibody or antigen-binding fragment thereof comprises an amino acid sequence having at least 90%, preferably at least 95%, and more preferably at least 98% homology to the amino acids of SEQ ID NO: 7, and having amino acids in which

any one amino acid from among glutamic acid at position 33 (corresponding to E33 of VL-CDR1), tyrosine at position 37 (corresponding to Y37 of VL-CDR1), valine at position 94 (corresponding to V94 of VL-CDR3), glutamine at position 95 (corresponding to Q95 of VL-CDR3), glycine at position 159 (corresponding to G159 of VH-CDR1), phenylalanine at position 160 (corresponding to F160 of VH-CDR1), phenylalanine at position 162 (corresponding to F162 of VH-CDR1), asparagine at position 164 (corresponding to N164 of VH-CDR1), glycine at position 233 (corresponding to G233 of VH-CDR3), tyrosine at position 235 (corresponding to Y235 of VH-CDR3), tyrosine at position 236 (corresponding to Y236 of VH-CDR3), aspartic acid at position 237 (corresponding to D237 of VH-CDR3), arginine at position 239 (corresponding to R239 of VH-CDR3), tyrosine at position 240, and tyrosine at position 242 (corresponding to Y240 or Y242 of VH-CDR3) numbered from the N-terminus in the amino acid sequence of VL-CDR1, VL-CDR2, VL-CDR3, VH-CDR1, VH-CDR2, or VH-CDR3 is substituted with alanine, or

(2) any one amino acid from among tyrosine at position 96 (corresponding to Y96 of VL-CDR3) and tyrosine at position 234 (Y234 of VH-CDR3) numbered from the N-terminus is substituted with phenylalanine.

According to yet another preferred aspect of the anti-DNP antibody of the present invention, the anti-DNP antibody or antigen-binding fragment thereof (also referred to below as the “anti-DNP antibody 5 or antigen-binding fragment 5 thereof”) comprises an amino acid sequence in which a substitution below is made in the amino acids of SEQ ID NO: 7:

(a) any one amino acid from among glutamic acid at position 33, tyrosine at position 37, valine at position 94, glutamine at position 95, glycine at position 159, phenylalanine at position 160, phenylalanine at position 162, asparagine at position 164, glycine at position 233, tyrosine at position 235, tyrosine at position 236, aspartic acid at position 237, arginine at position 239, tyrosine at position 240, and tyrosine at position 242 numbered from the N-terminus is substituted with alanine; or

(b) any one amino acid from among tyrosine at position 96 and tyrosine at position 234 numbered from the N-terminus is substituted with phenylalanine.

According to yet another preferred aspect of the anti-DNP antibody of the present invention, the anti-DNP antibody is an antibody or antigen-binding fragment thereof (also referred to below as the “anti-DNP antibody 6 or antigen-binding fragment 6 thereof”) in which the amino acid sequence thereof is represented by any of SEQ ID NO: 9 through 25 below.

Sequence No. 9:
MADYKDIVLTQSPSSLSASLGERVSLTCRSSQAISGYLGWLQQKPDGSIK
RLTYAASTLDSGVPKRFSGSRSGSDYSLTISSLESEDFADYYCVQYASYP
YTFGGGTKLEMKRGGGGSGGGGSGGGGSGGGGSQIQLQESGGGLVQPGGS
MKLSCVASGFTFSNYWMNWVRQSPEKGLDWVAEIRLKSNNYATHYAESVK
GRFTISRDDSKSSVYLQMNNLRAEDTGIYYCTGYYYDSRYGYWGQGTTVT
VSS
Sequence No. 10:
MADYKDIVLTQSPSSLSASLGERVSLTCRSSQEISGAYLGWLQQKPDGSI
KRLIYAASTLDSGVPKRFSGSRSGSDYSLTISSLESEDFADYYCVQYASY
PYTFGGGTKLEMKRGGGGSGGGGSGGGGSGGGGSQIQLQESGGGLVQPGG
SMKLSCVASGFTFSNYWMNWVRQSPEKGLDWVAEIRLKSNNYATHYAESV
KGRFTISRDDSKSSVYLQMNNLRAEDTGIYYCTGYYYDSRYGYWGQGTTV
TVSS
Sequence No. 11:
MADYKDIVLTQSPSSLSASLGERVSLTCRSSQEISGYLGWLQQKPDGSIK
RLIYAASTLDSGVPKRFSGSRSGSDYSLTISSLESEDFADYYCAQYASYP
YTFGGGTKLEMKRGGGGSGGGGSGGGGSGGGGSQIQLQESGGGLVQPGGS
MKLSCVASGFTFSNYWMNWVRQSPEKGLDWVAEIRLKSNNYATHYAESVK
GRFTISRDDSKSSVYLQMNNLRAEDTGIYYCTGYYYDSRYGYWGQGTTVT
VSS
Sequence No. 12:
MADYKDIVLTQSPSSLSASLGERVSLTCRSSQEISGYLGWLQQKPDGSIK
RLIYAASTLDSGVPKRFSGSRSGSDYSLTISSLESEDFADYYCVAYASYP
YTFGGGTKLEMKRGGGGSGGGGSGGGGSGGGGSQIQLQESGGGLVQPGGS
MKLSCVASGFTFSNYWMNWVRQSPEKGLDWVAEIRLKSNNYATHYAESVK
GRFTISRDDSKSSVYLQMNNLRAEDTGIYYCTGYYYDSRYGYWGQGTTVT
VSS
Sequence No. 13:
MADYKDIVLTQSPSSLSASLGERVSLTCRSSQEISGYLGWLQKKPDGSIK
RLIYAASTLDSGVPKRFSGSRSGSDYSLTISSLESEDFADYYCVQFASYP
YTFGGGTKLEMKRGGGGSGGGGSGGGGSGGGGSQIQLQESGGGLVQPGGS
MKLSCVASGFTFSNYWMNWVRQSPEKGLDWVAEIRLKSNNYATHYAESVK
GRFTISRDDSKSSVYLQMNNLRAEDTGIYYCTGYYYDSRYGYWGQGTTVT
VSS
Sequence No. 14:
MADYKDIVLTQSPSSLSASLGERVSLTCRSSQEISGYLGWLQQKPDGSIK
RLIYAASTLDSGVPKRFSGSRSGSDYSLTISSLESEDFADYYCVQYASYP
YTFGGGTKLEMKRGGGGSGGGGSGGGGSGGGGSQIQLQESGGGLVQPGGS
MKLSCVASAFTFSNYWMNWVRQSPEKGLDWVAEIRLKSNNYATHYAESVK
GRFTISRDDSKSSVYLQMNNLRAEDTGIYYCTGYYYDSRYGYWGQGTTVT
VSS
Sequence No. 15:
MADYKDIVLTQSPSSLSASLGERVSLTCRSSQEISGYLGWLQQKPEGSIK
RLIYAASTLDSGVPKRFSGSRSGSDYSLTISSLESEDFADYYCVQYASYP
YTFGGGTKLEMKRGGGGSGGGGSGGGGSGGGGSQIQLQESGGGLVQPGGS
MKLSCVASGATFSNYWMNWVRQSPEKGLDWVAEIRLKSNNYATHYAESVK
GRFTISRDDSKSSVYLQMNNLRAEDTGIYYCTGYYYDSRYGYWGQGTTVT
VSS
Sequence No. 16:
MADYKDIVLTQSPSSLSASLGERVSLTCRSSQEISGYLGWLQQKPDGSIK
RLIYAASTLDSGVPKRFSGSRSGSDYSLTISSLESEDFADYYCVQYASYP
YTFGGGTKLEMKRGGGGSGGGGSGGGGSGGGGSQIQLQESGGGLVQPGGS
MKLSCVASGFTASNYWMNWVRQSPEKGLDWVAEIRLKSNNYATHYAESVK
GRFTISRDDSKSSVYLQMNNLRAEDTGIYYCTGYYYDSRYGYWGQGTTVT
VSS
Sequence No. 17:
MADYKDIVITQSPSSLSASLGERVSLTCRSSQEISGYLGWLQQKPDGSIK
RLIYAASTLDSGVPKRFSGSRSGSDYSLTISSLESEDFADYYCVQYASYP
YTFGGGTKLEMKRGGGGSGGGGSGGGGSGGGGSQIQLQESGGGLVQPGGS
MKLSCVASGFTFSAYWMNWVRQSPEKGLDWVAEIRLKSNNYATHYAESVK
GRFTISRDDSKSSVYLQMNNLRAEDTGIYYCTGYYYDSRYGYWGQGTTVT
VSS
Sequence No. 18:
MADYKDIVLTQSPSSLSASLGERVSLTCRSSQEISGYLGWLQQKPDGSIK
RLIYAASTLDSGVPKRFSGSRSGSDYSLTISSLESEDFADYYCVQYASYP
YTFGGGTKLEMKRGGGGSGGGGSGGGGSGGGGSQIQLQESGGGLVQPGGS
MKLSCVASGFTFSNYWMNWVRQSPEKGLDWVAEIRLKSNNYATHYAESVK
GRFTISRDDSKSSVYLQMNNLRAEDTGIYYCTAYYYDSRYGYWGQGTTVT
VSS
Sequence No. 19:
MADYKDIVLTQSPSSLSASLGERVSLTCRSSQEISGYLGWLQQKPDGSIK
RLIYAASTLDSGVPKRFSGSRSGSDYSLTISSLESEDFADYYCVQYASYP
YTFGGGTKLEMKRGGGGSGGGGSGGGGSGGGGSQIQLQESGGGLVQPGGS
MKLSCVASGFTFSNYWMNWVRQSPEKGLDWVAEIRLKSNNYATHYAESVK
GRFTISRDDSKSSVYLQMNNLRAEDTGIYYCTGFYYDSRYGYWGQGTTVT
VSS
Sequence No. 20:
MADYKDIVLTQSPSSLSASLGERVSLTCRSSQEISGYLGWLQKKPDGSIK
RLIYAASTLDSGVPKRFSGSRSGSDYSLTISSLESEDFADYYCVQYASYP
YTFGGGTKLEMKRGGGGSGGGGSGGGGSGGGGSQIQLQESGGGLVQPGGS
MKLSCVASGFTFSNYWMNWVRQSPEKGLDWVAEIRLKSNNYATHYAESVK
GRFTISRDDSKSSVYLQMNNLRAEDTGIYYCTGYAYDSRYGYWGQGTTVT
VSS
Sequence No. 21:
MADYKDIVLTQSPSSLSASLGERVSLTCRSSQEISGYLGWLQQKPDGSIK
RLIYAASTLDSGVPKRFSGSRSGSDYSLTISSLESEDFADYYCVQYASYP
YTFGGGTKLEMKRGGGGSGGGGSGGGGSGGGGSQIQLQESGGGLVQPGGS
MKLSCVASGFTFSNYWMNWVRQSPEKGLDWVAEIRLKSNNYATHYAESVK
GRFTISRDDSKSSVYLQMNNLRAEDTGIYYCTGYYADSRYGYWGQGTTVT
VSS
Sequence No. 22:
MADYKDIVLTQSPSSLSASLGERVSLTCRSSQEISGYLGWLQQKPDGSIK
RLIYAASTLDSGVPKRFSGSRSGSDYSLTISSLESEDFADYYCVQYASYP
YTFGGGTKLEMKRGGGGSGGGGSGGGGSGGGGSQIQLQESGGGLVQPGGS
MKLSCVASGFTFSNYWMNWVRQSPEKGLDWVAEIRLKSNNYATHYAESVK
GRFTISRDDSKSSVYLQMNNLRAEDTGIYYCTGYYYASRYGYWGWGTTVT
VSS
Sequence No. 23:
MADYKDIVLTQSPSSLSASLGERVSLTCRSSQEISGYLGWLQQKPDGSIK
RLIYAASTLDSGVPKRFSGSRSGSDYSLTISSLESEDFADYYCVQYASYP
YTFGGGTKLEMKRGGGGSGGGGSGGGGSGGGGSQIQLQESGGGLVQPGGS
MKLSCVASGFTFSNYWMNWVRQSPEKGLDWVAEIRLKSNNYATHYAESVK
GRFTISRDDSKSSVYLQMNNLRAEDTGIYYCTGYYYDSAYGYWGQGTTVT
VSS
Sequence No. 24:
MADYKDIVLTQSPSSLSASLGERVSLTCRSSQEISGYLGWLQQKPDGSIK
RLIYAASTLDSGVPKRFSGSRSGSDYSLTISSLESEDFADYYCVQYASYP
YTFGGGTKLEMKRGGGGSGGGGSGGGGSGGGGSQIQLQESGGGLVQPGGS
MKLSCVASGFTFSNYWMNWVRQSPEKGLDWVAEIRLKSNNYATHYAESVK
GRFTISRDDSKSSVYLQMNNLRAEDTGIYYCTGYYYDSRAGYWGQGTTVT
VSS
Sequence No. 25:
MADYKDIVLTQSPSSLSASLGERVSLTCRSSQEISGYLGWLQQKPDGSIK
RLIYAASTLDSGVPKRFSGSRSGSDYSLTISSLESEDFADYYCVQYASYP
YTFGGGTKLEMKRGGGGSGGGGSGGGGSGGGGSQIQLQESGGGLVQPGGS
MKLSCVASGFTFSNYWMNWVRQSPEKGLDWVAEIRLKSNNYATHYAESVK
GRFTISRDDSKSSVYLQMNNLRAEDTGIYYCTGYYYDSRYGAWGQGTTVT
VSS

Another embodiment of the present invention is an isolated nucleic acid coding for any of the anti-DNP antibodies or antigen-binding fragments thereof described above (specifically, anti-DNP antibodies 1 through 5 and the antigen-binding fragments 1 through 5 thereof).

According to a preferred aspect of the nucleic acid of the present invention, the nucleic acid comprises a base sequence represented by SEQ ID NO: 8 below.

Sequence No. 8:
ATGGCGGACTACAAAGACATTGTGCTGACCCAGTCTCCATCCTCTTTATC
TGCCTCTCTGGGAGAAAGAGTCAGTCTCACTTGTCGGTCAAGTCAGGAAA
TTAGTGGTTACTTAGGCTGGCTTCAGCAGAAACCAGATGGAAGTATTAAA
CGCCTGATCTACGCCGCATCCACTTTAGATTCTGGTGTCCCAAAAAGGTT
CAGTGGCAGTAGGTCTGGGTCAGATTATTCTCTCACCATCAGCAGCCTTG
AGTCTGAAGATTTTGCAGACTATTATTGTGTACAATATGCTAGTTATCCG
TACACGTTCGGAGGGGGGACCAAGCTGGAAATGAAACGCGGTGGTGGTGG
TTCTGGTGGTGGTGGTTCTGGCGGCGGCGGCTCCGGTGGTGGTGGATCCC
AGATTCAGCTTCAGGAGTCTGGAGGAGGCTTGGTGCAACCTGGAGGATCC
ATGAAACTCTCCTGTGTTGCCTCTGGATTCACTTTCAGTAACTACTGGAT
GAACTGGGTCCGCCAGTCTCCAGAGAAGGGGCTTGACTGGGTTGCTGAAA
TTAGATTGAAATCTAATAATTATGCAACACATTATGCGGAGTCTGTGAAA
GGGAGGTTCACCATCTCAAGAGATGATTCCAAAAGTAGTGTCTACCTGCA
AATGAACAACTTAAGAGCTGAAGACACTGGCATTTATTACTGTACCGGTT
ATTACTACGATAGTAGGTACGGCTACTGGGGCCAAGGCACCACGGTACCC
GTCTCCTCGGCCTCG

Another embodiment of the present invention is a plasmid or vector including the nucleic acid of the present invention.

Another embodiment of the present invention is a fluorescent probe used in the method for fluorescently labeling an intracellular protein of the present invention, the fluorescent probe including a compound represented by formula (I) or formula (Ia) below or a salt thereof.

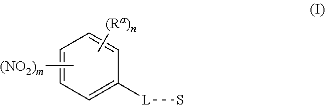

In formula (I): S is a fluorescent group, L is a linker, and R^{a} is a monovalent substituent; m is an integer of 0 to 2, and n is an integer of 0 to 2; when m is 2, n is 0; when m is 1, n is 1 or 0; when m is 0, n is 2; and when n is 2, the monovalent substituents of R^{a} may be the same or different.

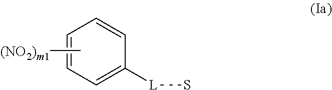

In formula (Ia), S is a fluorescent group, L is a linker, and m1 is 1 or 2.

The compounds or salts thereof (compounds of the present invention) represented by formulas (I) and (Ia) are as described above.

Non-limiting examples of the compound of the present invention are shown below.

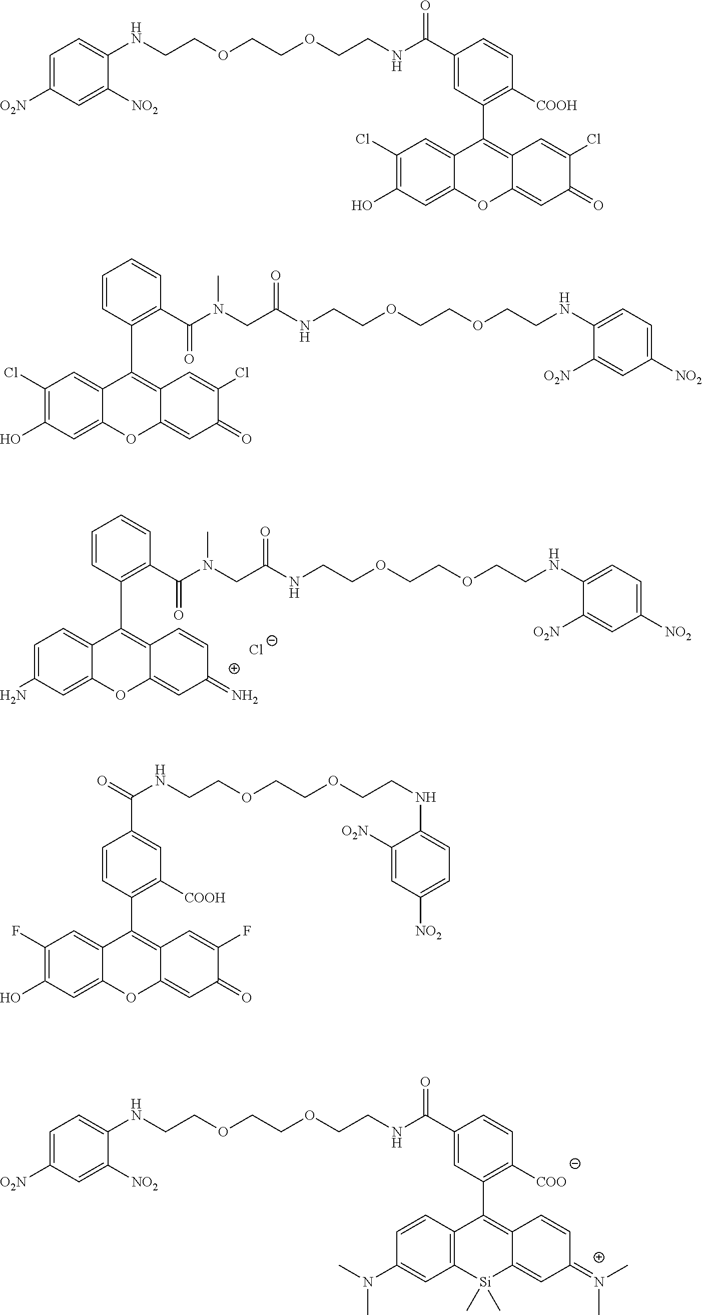

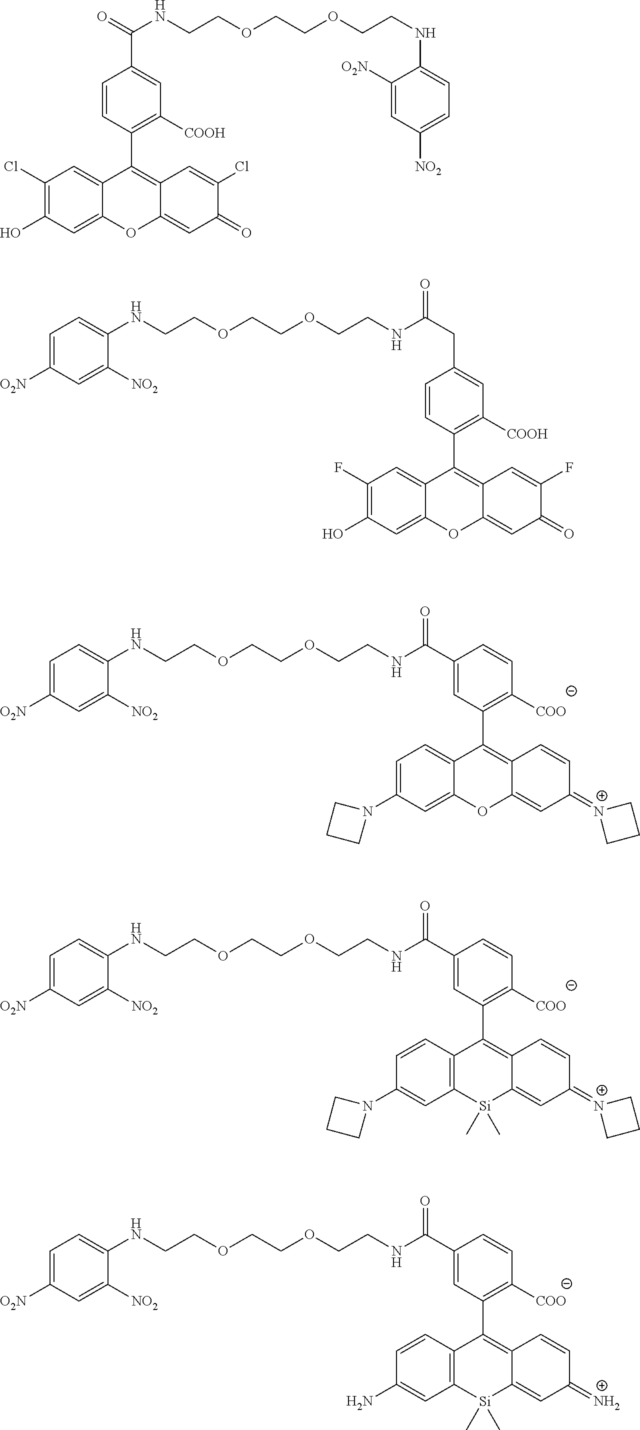

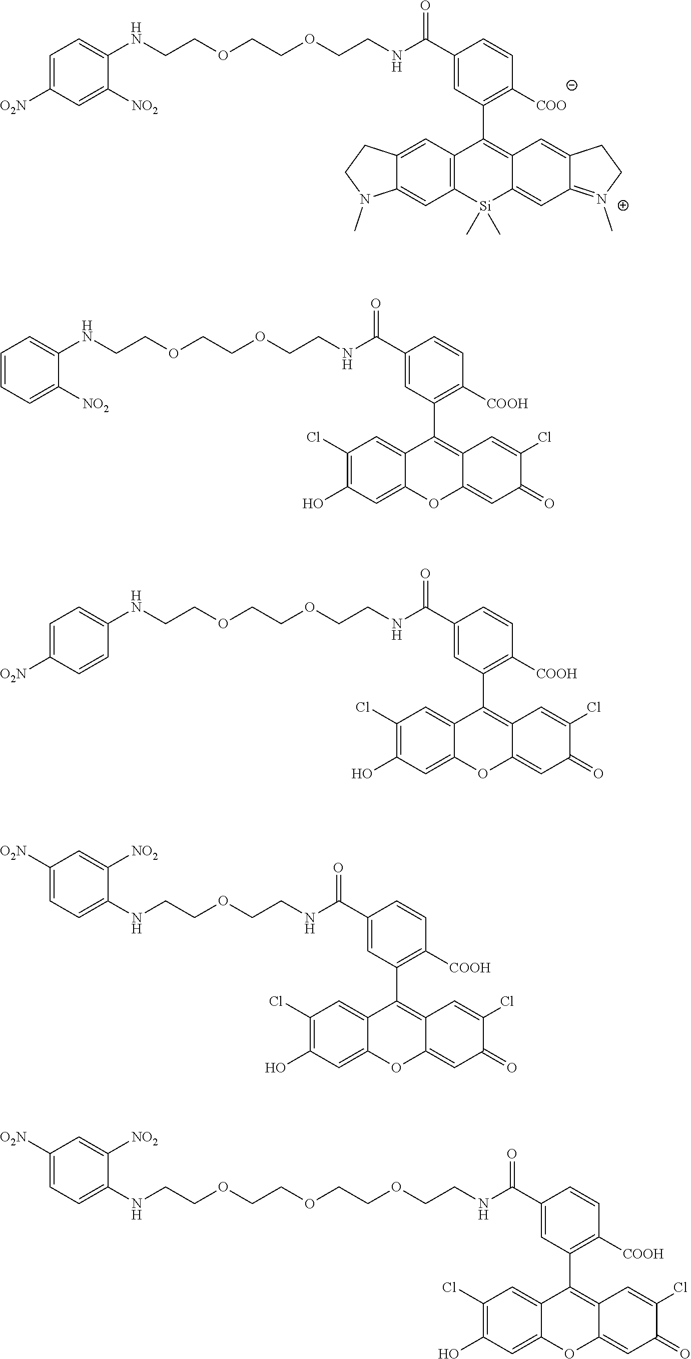

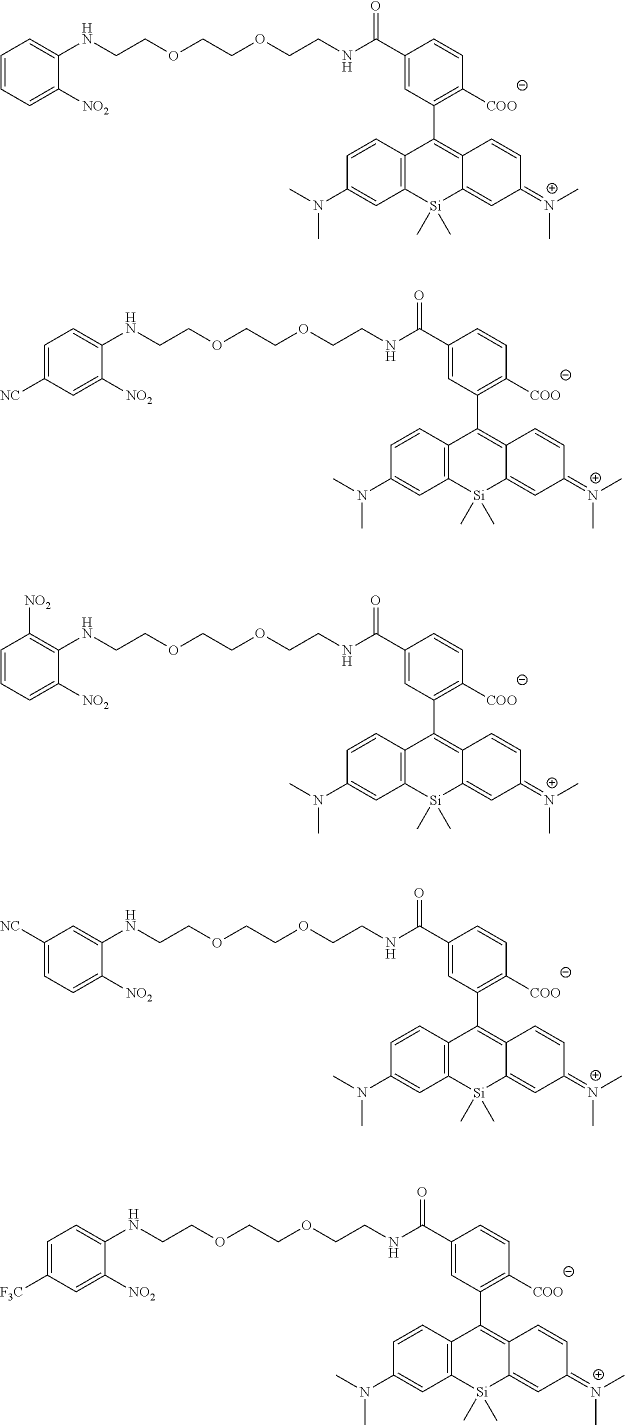

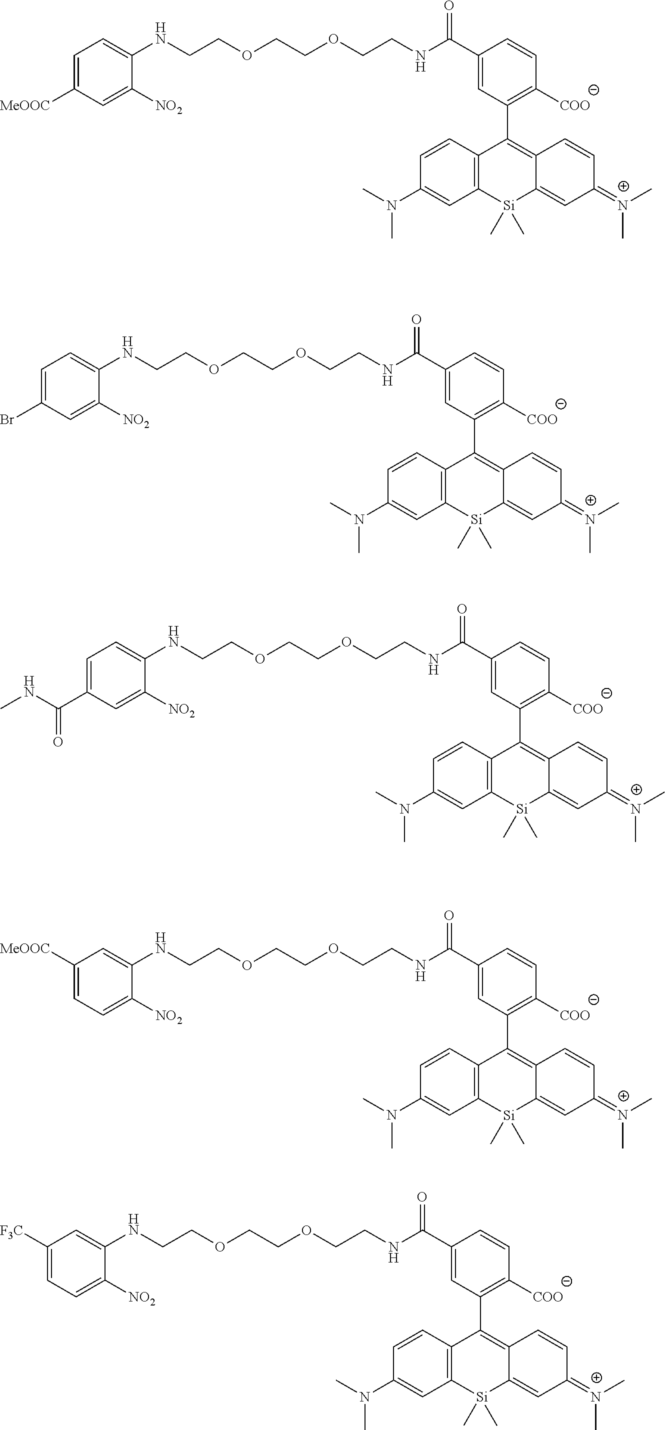

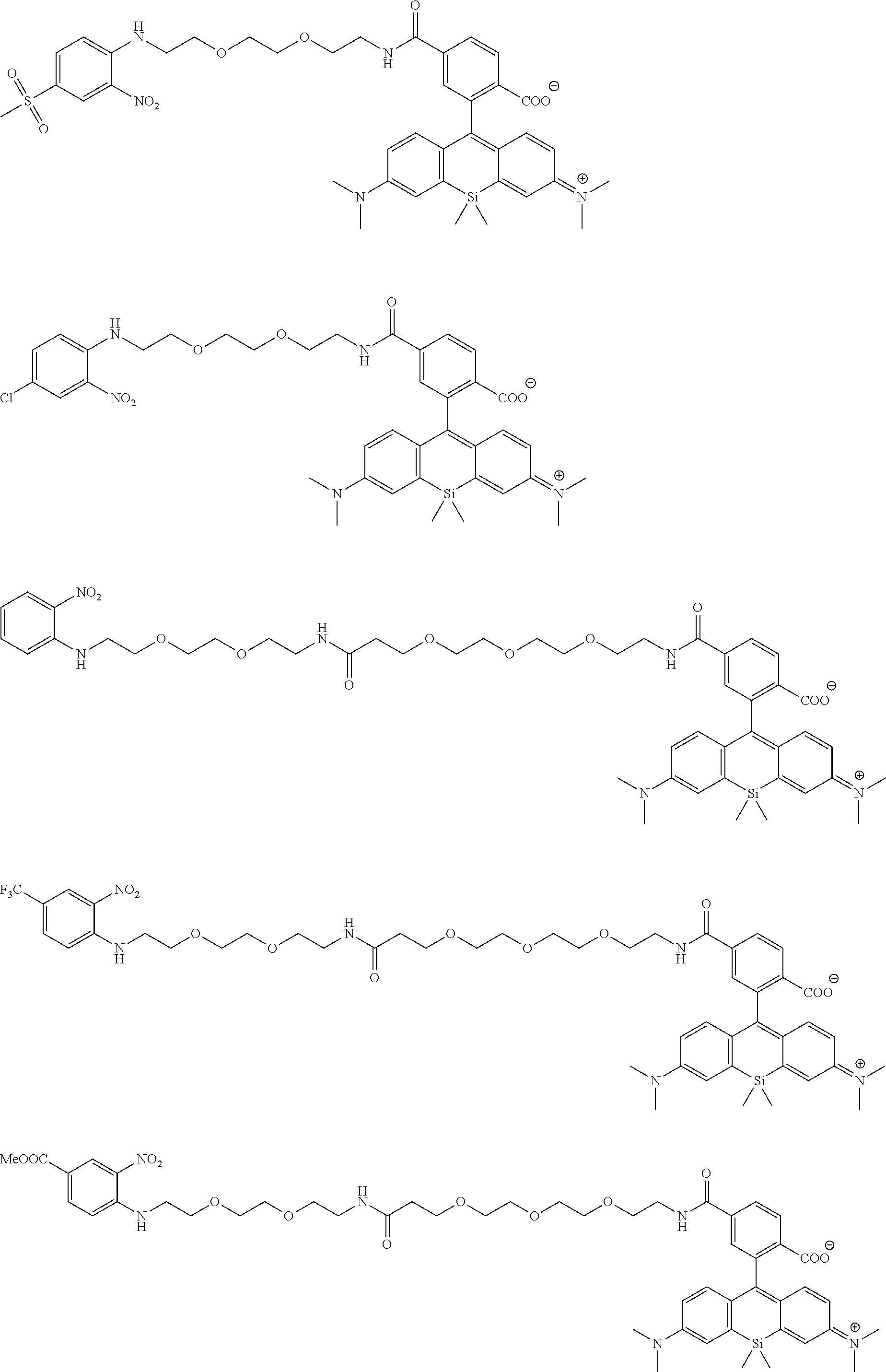

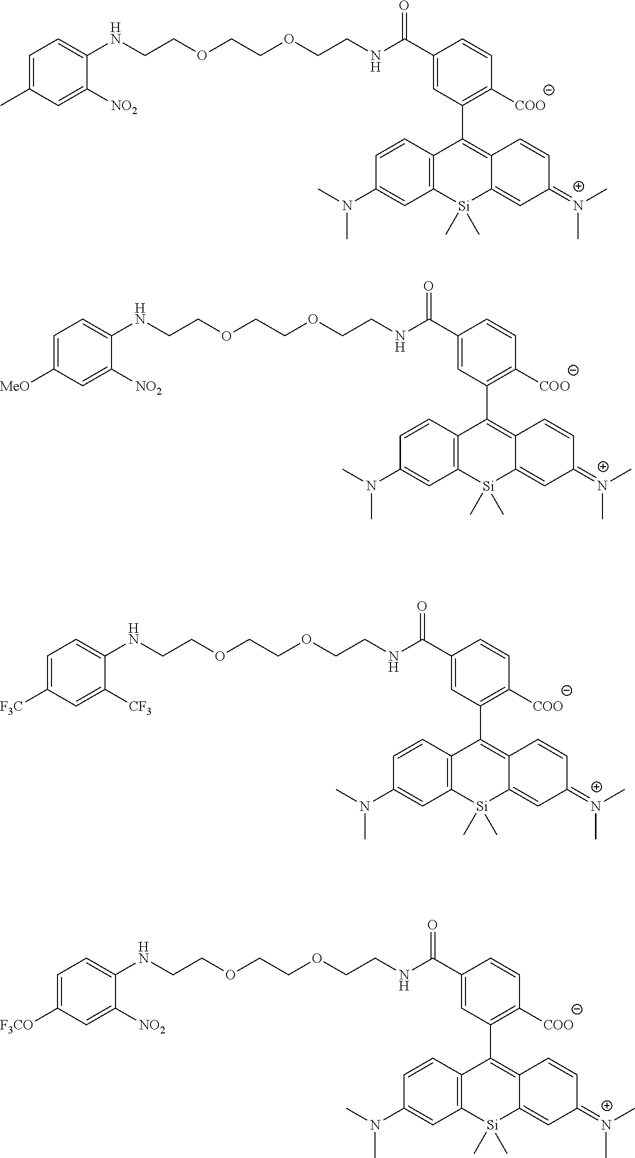

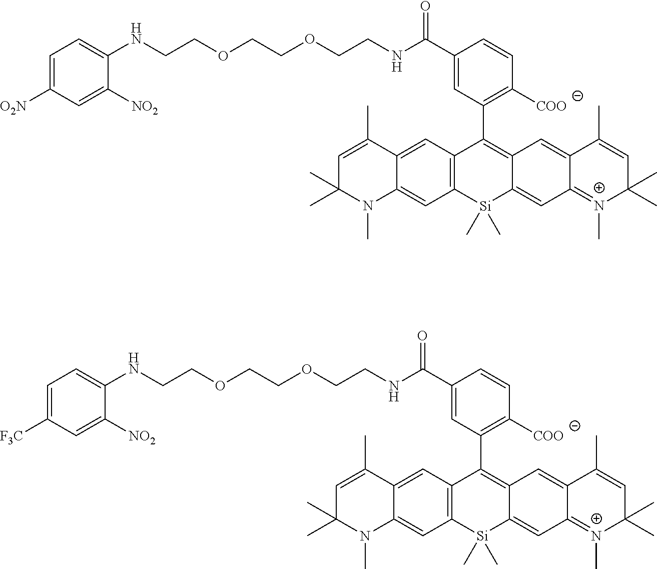

Depending on the type of substituent, the compound of the present invention sometimes has one or more asymmetric carbons, and an optical isomer or a diastereoisomer or other stereoisomer is sometimes present. Stereoisomers in pure form, any mixture of stereoisomers, and racemates and the like are all included in the scope of the present invention. The compound or salt thereof of the present invention represented by general formula (I) also sometimes exists as a hydrate or a solvate, but these substances are all included in the scope of the present invention. The type of solvent for forming a solvate is not particularly limited, but ethanol, acetone, isopropanol, and other solvents can be cited as examples thereof.

Methods for manufacturing a typical example of the compound of the present invention are specifically presented in examples of the present specification. Consequently, on the basis of the description given in the examples, a person skilled in the art could select appropriate raw materials for reaction, reaction conditions, reagents for reaction, and the like and modify or change the methods as needed, and thereby manufacture the compound of the present invention represented by general formula (I).

Methods for using the fluorescent probe of the present invention are not particularly limited, and the fluorescent probe of the present invention can be used in the same manner as a conventional and publicly known fluorescent probe. Usually, the compounds or salts thereof of the present invention are dissolved in physiological saline, a buffer solution, or another aqueous medium, or a mixture of ethanol, acetone, ethylene glycol, dimethyl sulfoxide, dimethyl formamide, or another water-miscible organic solvent and an aqueous medium, the solution is added to an appropriate buffer solution including a tissue or cell in which the fusion protein described above is expressed, and a fluorescent spectrum may be measured. The fluorescent probe of the present invention may be combined with an appropriate additive and used in the form of a composition. The fluorescent probe can be combined with a buffer, a solubilizer, a pH regulator, or other additive, for example.

Another embodiment of the present invention is a super-resolution imaging method including obtaining, in a cell, a fusion protein of a labeling object protein and an anti-DNP (dinitrophenyl compound) antibody, bringing a compound represented by formula (I) or a salt thereof into contact with the cell, and fluorescently labeling the object protein by reacting the fusion protein and the compound represented by formula (I) or a salt thereof.

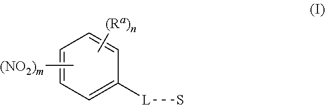

In formula (I), S, L, R^{a}, m, and n are as described above.

The super-resolution imaging method of the present invention preferably uses single-molecule localization microscopy.

A super-resolution imaging method using single-molecule localization microscopy can be performed on the basis of the disclosure in non-patent literature 13 (M. J. Rust, M. Bates, X. Zhuang, Sub-diffraction-limit imaging by stochastic optical reconstruction microscopy (STORM). Nat Methods 3, 793-795 (2006)) and non-patent literature 14(M. Heilemann, S. van de Linde, M. Schuettpelz, R. Kasper, B. Seefeldt, A. Mukherjee, P. Tinnefeld, M. Sauer, Subdiffraction-resolution fluorescence imaging with conventional fluorescent probes. Angew Chem Int Ed Engl 47, 6172-6176 (2008)), for example.

According to a preferred aspect of the super-resolution imaging method of the present invention, the anti-DNP antibody in the fusion protein is an anti-DNP antibody or antigen-binding fragment thereof which comprises an amino acid sequence having at least 90%, preferably at least 95%, and more preferably at least 98% homology to the amino acids of SEQ ID NO: 7 and includes the amino acid sequences represented by SEQ ID NO: 1 through 6, and which comprises an amino acid sequence in which at least one, preferably one, of the substitutions below is made in the amino acid sequence represented by any of SEQ ID NO: 1 through 6:

(a) any one amino acid from among glutamic acid at position 33, tyrosine at position 37, valine at position 94, glutamine at position 95, glycine at position 159, phenylalanine at position 160, phenylalanine at position 162, asparagine at position 164, glycine at position 233, tyrosine at position 235, tyrosine at position 236, aspartic acid at position 237, arginine at position 239, tyrosine at position 240, and tyrosine at position 242 numbered from the N-terminus is substituted with alanine; or

(b) any one amino acid from among tyrosine at position 96 and tyrosine at position 234 numbered from the N-terminus is substituted with phenylalanine.

Through use of an anti-DNP antibody or antigen-binding fragment thereof comprising the amino acid sequence described above as the anti-DNP antibody, it is possible to increase binding/dissociation kinetics (k_{off}) of a QODE probe and a molecular tag (De-QODE tag) in which quenching is removed and fluorescence is turned ON by binding of the molecular tag with an anti-quencher antibody, and to realize highly practical super-resolution imaging.

According to a preferred aspect of the super-resolution imaging method of the present invention, the anti-DNP antibody in the fusion protein is an anti-DNP antibody or antigen-binding fragment thereof comprising an amino acid sequence in which a substitution below is made in the amino acids of SEQ ID NO: 7:

(1) any one amino acid from among glutamic acid at position 33, tyrosine at position 37, valine at position 94, glutamine at position 95, glycine at position 159, phenylalanine at position 160, phenylalanine at position 162, asparagine at position 164, glycine at position 233, tyrosine at position 235, tyrosine at position 236, aspartic acid at position 237, arginine at position 239, tyrosine at position 240, and tyrosine at position 242 numbered from the N-terminus is substituted with alanine; or

(2) any one amino acid from among tyrosine at position 96 and tyrosine at position 234 numbered from the N-terminus is substituted with phenylalanine.

Another embodiment of the present invention is a fluorescent probe used in the super-resolution imaging method of the present invention, the fluorescent probe including a compound represented by formula (I) below or a salt thereof.

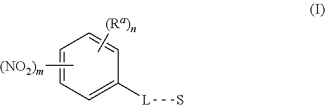

In formula (I): S is a fluorescent group, L is a linker, and R^{a} is a monovalent substituent; m is an integer of 0 to 2, and n is an integer of 0 to 2; when m is 2, n is 0; when m is 1, n is 1 or 0; when m is 0, n is 2; and when n is 2, the monovalent substituents of R^{a} may be the same or different.

The monovalent substituent represented by R^{a} is selected from the group consisting of a halogen atom, a C1-10 alkyl group, a C1-10 alkoxy group, a cyano group, an ester group, an amide group, an alkyl sulfonyl group, a C1-10 alkyl group in which at least one hydrogen atom is substituted with a fluorine atom, and a C1-10 alkoxy group in which at least one hydrogen atom is substituted with a fluorine atom.

Another embodiment of the present invention is a fluorescent probe used in the super-resolution imaging method of the present invention, the fluorescent probe including a compound represented by formula (Ib) below or a salt thereof.

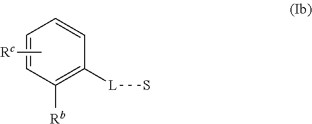

In formula (Ib), S is a fluorescent group, L is a linker, and R^{b} and R^{c} are selected from combinations below. (R^{b},R^{c}):(NO₂, p-NO₂), (NO₂, p-Br), (NO₂,p-SO₂Me), (NO₂, p-C), (NO₂, m-CN). (NO₂, p-CN), (NO₂, p-COOMe), (CF₃, p-CF₃), (NO₂, p-CONHMe), (NO₂, m-COOMe) (NO₂, H)

(Here, p- and m- represent R^{c} being in a para position and a meta position on the benzene ring, respectively, with respect to L.)

In formula (Ib), S is preferably represented by formula (III) below.

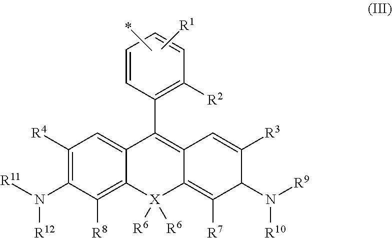

In formula (III), R¹-R⁸ and X are as described for formula (II).

In formula (Ib), L can be represented by T-Y, where Y is a bonding group for bonding with the fluorescent group S, and T represents a crosslinking group.

The bonding group represented by Y is selected from an amide group (—CONH—, —CONR′—, —R—CONH—, or —R—CONR′—), an alkylamide group (—CONH—R— or —CONR′—R—), an ester group (—COO—), an alkylester group (—R—COO— or —COO—R—), a carbonylamino group (—NHCO— or —NR′CO—), or an alkylether group (—RO— or —OR—). In these groups, R represents a divalent hydrocarbon group, preferably a C1-10 alkylene group, and more preferably a C1-5 alkylene group, and R′ represents a C1-5 alkyl.

Any crosslinking group which works as a spacer for connecting the bonding group Y and the benzene ring of the compound of formula (Ib) can be used as the crosslinking group T. Examples thereof include, but are not limited to, substituted or unsubstituted divalent hydrocarbon groups (alkanes, alkenes, alkynes, cycloalkanes, aromatic hydrocarbons, and the like), dialkylether groups (e.g., dimethyl ether, diethyl ether, methylethyl ether, and the like), an ethylene glycol group, a diethylene glycol group, a triethylene glycol group, a polyethylene glycol group, an amide group, a carbonyl or the like, and heterocyclic groups (e.g., a divalent piperidine ring or the like), and combinations of two or more of the above groups. The crosslinking group may have, at one or both ends thereof, a functional group capable of bonding to Y and the benzene ring of the compound of formula (Ib), examples of such a functional group including an amino group, an alkylamino group, an aminoalkyl group, a carbonyl group, a carboxyl group, an amide group, an alkylamide group, and the like.

The crosslinking group T also includes a group represented by the formula T₁-(W)-T₂. Each of the crosslinking groups presented as examples above can be used as T₁ and T₂. The group W, when present, is a group for connecting T₁ and T₂, and examples thereof include an amino group, an alkylamino group, an aminoalkyl group, a carbonyl group, a carboxyl group, an amide group, an alkylamide group, and the like.

Examples of such a crosslinking group include, but are not limited to, a group in which a triethylene glycol group and a diethylene glycol group are bonded via an amide group, an alkylamide group, or the like. Furthermore, the crosslinking group represented by the formula T₁-(W)-T₂ may have, at one or both ends thereof, a functional group (e.g., an amino group, an alkylamino group, an aminoalkyl group, a carbonyl group, a carboxyl group, an amide group, an alkylamide group, or the like) capable of bonding to Y and the benzene ring of the compound of formula (Ib).

A preferred aspect of the present invention is a fluorescent probe used in the super-resolution imaging method of the present invention, the fluorescent probe including a compound below or a salt thereof.

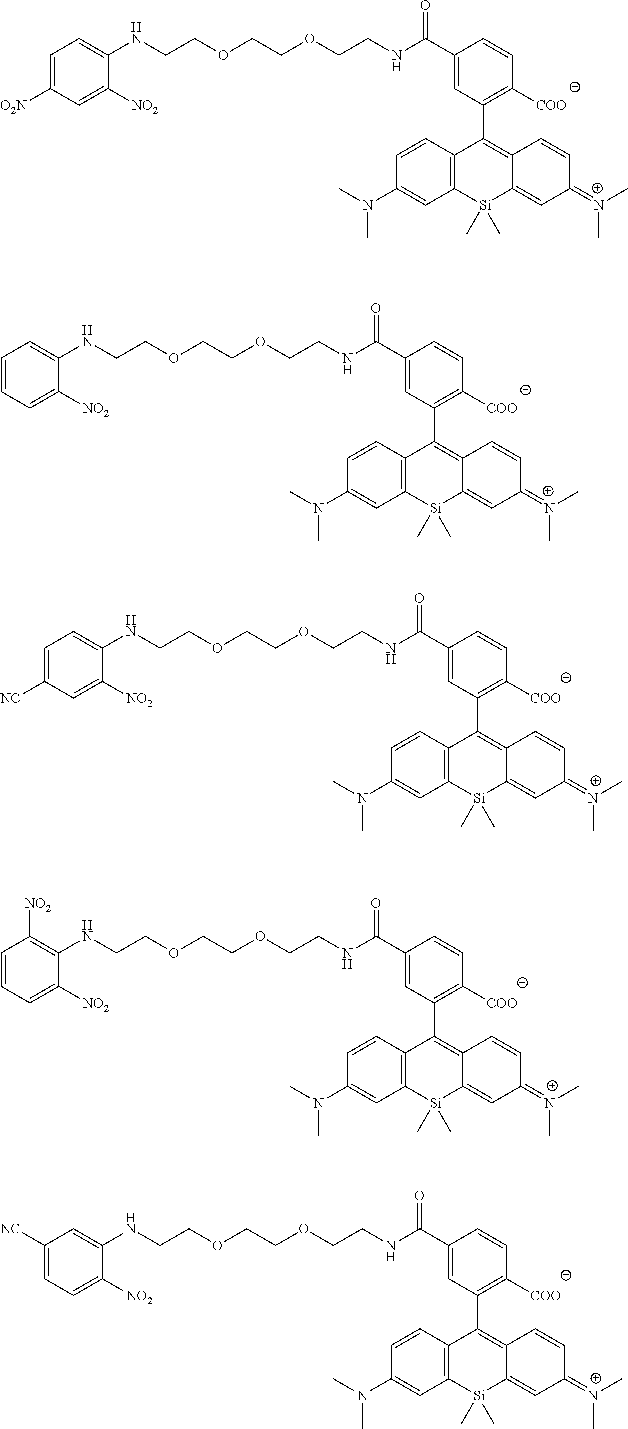

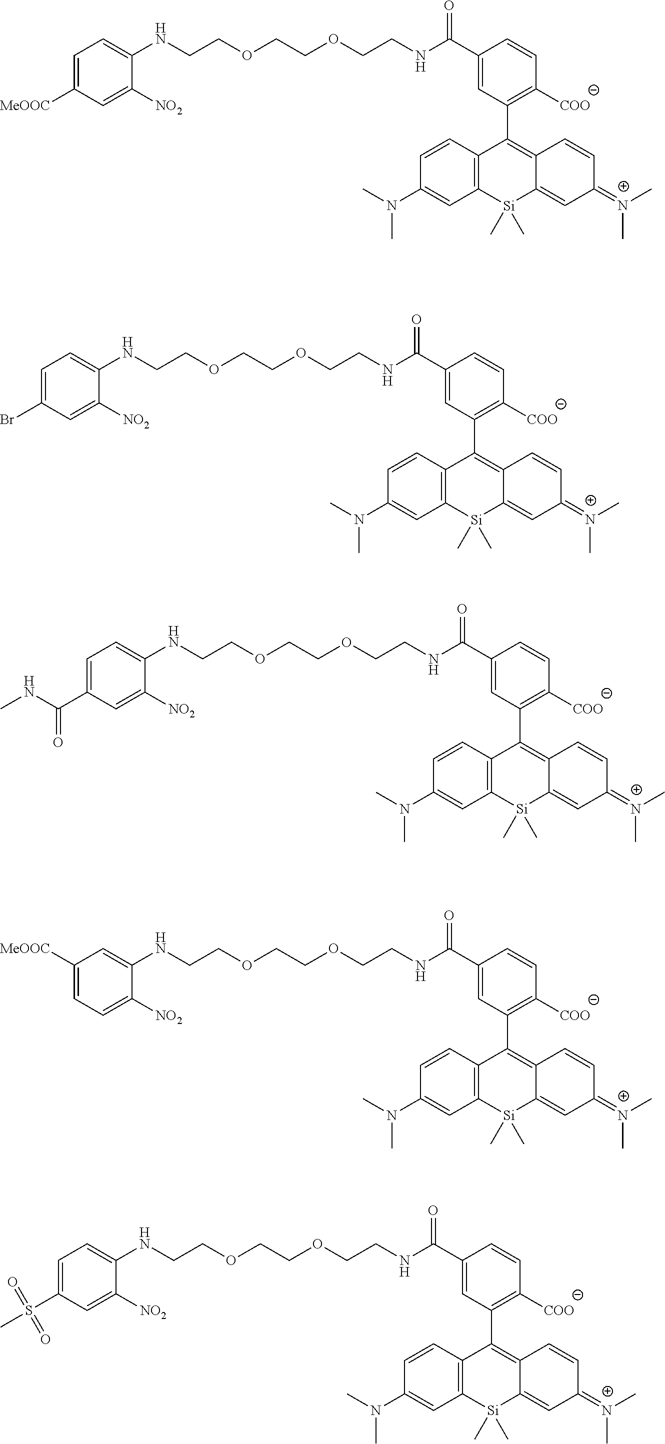

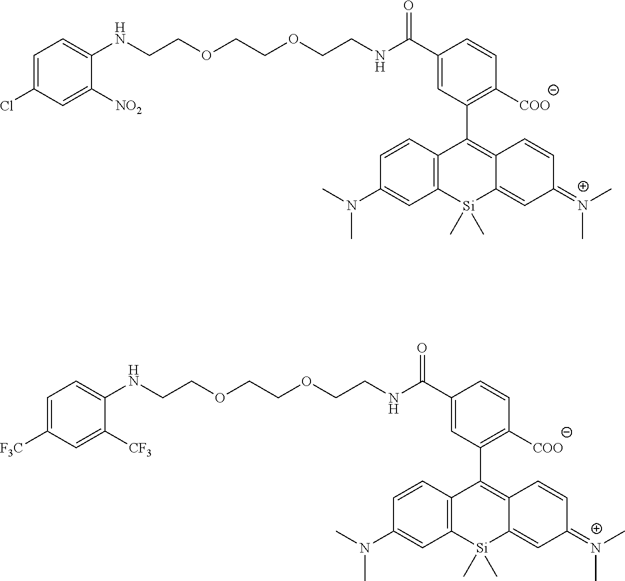

Another embodiment of the present invention is a fluorescent probe used in the fluorescent labeling method of the present invention, the fluorescent probe including the compound of the present invention or a salt thereof, and a kit for a protein fluorescent labeling method, the kit including the plasmid or vector used in the fluorescent labeling method of the present invention.

The fluorescent labeling method kit of the present invention can be suitably used in the super-resolution imaging method.

### EXAMPLES

The present invention is illustrated but not limited by the following examples.

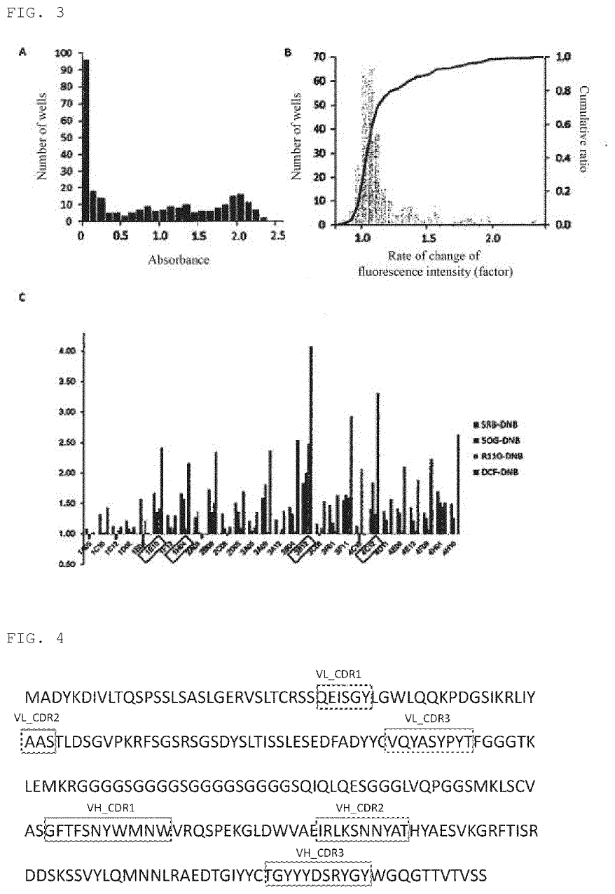

In the present example, development of a molecular tag technique for enabling ON/OFF control of fluorescence was advanced through the process illustrated in FIG. 2. Acquisition of an anti-DNP scFv clone capable of being expressed in a cell and synthesis of a fluorophore-DNP pair for significantly increasing fluorescence intensity by binding with the anti-DNP scFv clone were advanced in parallel. A fluorophore-DNP pair and anti-DNP scFv combination was obtained for which a significant fluorescence increase was exhibited in cultured cells expressing the anti-DNP scFv when loaded with the fluorophore-DNP pair. The propriety of application to fluorescence imaging in the cultured cells was investigated using the obtained combination.

1. Experimental Methods

[Acquisition of Anti-Dinitrophenol Monoclonal Antibody]

A complex of keyhole limpet hemocyanin (KLH) labeled with NHS-dinitrophenol was used as an antigen. The NHS-dinitrophenol and KLH were reacted for two hours at room temperature, and unreacted NHS-DNP was then removed using a NAP5 column (GE). An emulsion was prepared by mixing a dinitrophenol KLH conjugate and Freund's complete adjuvant, and five BALB/c mice (9-week-old females) were immunized with 0.1 mL each of the emulsion at a tail base thereof. At 19 days after immunization, lymph nodes were extracted from the mice, B cells were acquired by crushing the lymph nodes, and the B cells were suspended in 1000 μL of Lab Bunker (JUJI-FIELD), after which 500 μL of the suspension was dispensed into each of two cryotubes and stored at −80° C.

Recovered lymph node cells and myeloma cells (SP2, RIKEN BRC) were fused using GenomONE-CF (ISHIHARA SANGYO). The cell suspension after cell fusion was diluted with 44 mL of HAT medium (WAKO PURE CHEMICAL) including 10% BM Condimed H1 (ROCHE) and 10% serum and seeded on four 96-well plates (CORNING), and then cultured in 5% carbon dioxide at 37° C. At seven days after culturing, an antibody titer was evaluated using 30 μL of culture supernatant from each well. Cells from wells exhibiting increased fluorescence intensity of SRB-DNP and a high antibody titer in a hybridoma cell supernatant were selected, monoclonization thereof by limiting dilution was performed twice, and a hybridoma line was established.

[Evaluation of Antibody Titer by ELISA]

A conjugate of dinitrophenol and BSA was used as an antigen in screening. A 10 μg/mL BSA-DNP conjugate solution was added 100 μL at a time to an ELISA plate (Nunc), and adsorption was carried out for two hours at 37° C. After adsorption, the antigen solution was removed by washing with PBS, 30 μL of a hybridoma culture supernatant was added, and the plate was left for two hours at 37° C. After washing was performed three times with 200 μL of PBS, reaction was performed for 30 minutes at 37° C. with horseradish-peroxidase-labeled anti-mouse IgG antibodies. Washing with 200 μL of PBS was performed three times, and then 50 μL of a TMB color development kit (NACALAI TESQUE) was added and color development was performed. After color development, 50 μL of 1 M sulfuric acid was added and reaction was stopped, and absorbance at 450 nm was measured by a microplate reader (TECAN) using a reference wavelength of 590 nm.

[Conversion of Anti-DNP Monoclonal Antibody to Single-Chain Antibody (scFv)]

Anti-DNP monoclonal antibody-producing hybridomas in the amount of 10⁶ cells were recovered, and total RNA was acquired using RNeasy (Qiagen). With the resultant total RNA as a template, cDNA synthesis was performed using a PrimeScript RT Reagent Kit (Perfect Real Time) (TAKARA). With the resultant cDNA as a template, PCR using a degenerate primer (Kontermann, S. D. R., Antibody Engineering, Springer 1, (2010)) was performed, and cDNA fragments coding for light chain and heavy chain regions of each anti-DNP monoclonal antibody were amplified. The resultant cDNA fragments were then purified using a FastGene Gel/PCR Extraction Kit (NIPPON GENETICS), and cDNA fragments in which light chains and heavy chains are joined were acquired using overlap PCR. The resultant cDNA fragments were subcloned into a pAK400 vector (A. Krebber et al., Reliable cloning of functional antibody variable domains from hybridomas and spleen cell repertoires employing a reengineered phage display system. Journal of immunological methods 201, 35-55 (1997).) via SfiI restriction enzyme sites added to both ends of the resultant cDNA fragments. A cDNA sequence coding for the scFv was analyzed.

[MBP Fusion Protein Expression Construct]

The pAK400-scFv was HindIII digested and then smoothed using Klenow fragments (TAKARA). Furthermore, scFv cDNA fragments obtained by NcoI digestion were subcloned into NcoI/EcoRV-digested pMalc5E (pMalc5E-scFv).

[Expression and Purification of Anti-DNP scFv]

The anti-DNP scFv was expressed and purified as a fusion protein of maltose-binding protein. Escherichia coli BL21 (DE3) was transformed with the pMalc5E vector (pMalc5E-scFv) into which the purification object scFv sequence was introduced, and was cultured overnight on an LB medium plate including 100 μg/mL of ampicillin. A single colony was picked up and cultured overnight in 5 mL of a liquid LB medium including 100 μg/mL of ampicillin, and 1 mL of the resultant culture liquid was transferred to 100 mL of LB medium including 100 μg/mL of ampicillin. Shake culturing was performed at 200 rpm and a temperature of 37° C. until an optical density at 600 nm of 0.8 was reached, and after shake culturing was performed for 30 minutes at 15° C., IPTG was added to give a final concentration of 0.5 mM, and shake culturing was continued overnight. The E. coli were recovered after culturing and were disrupted using a sonicator. A supernatant was recovered by centrifuging (3000 g) an E. coli disruption liquid and purified with TALON His-tag affinity beads (TAKARA BIO), and 250 μL of a purified protein was obtained. An eluate was replaced with PBS, and yield was quantified by the Bradford method, after which the eluate was subjected to the measurements described below.

[Preparation of Animal Cell Expression Construct]

Preparation of Cytoplasmic Expression Construct

A vector was constructed to cause a fusion protein of the anti-DNP scFv and an infrared fluorescent protein TagRFP to be expressed in an animal cell. A BglII site was added to a forward primer and an EcoRI site was added to a reverse primer, and PCR was performed using pMalc5E-scFv as the template. The PCR product was digested with BglII and EcoRI, and then subcloned into the BglII/EcoRI sites of pTagRFP-C(EVROGEN) (pTagRFP-scFv). The pTagRFP-scFv was digested with NheI/BspEI and the cDNA sequence of TagRFP was excised, after which ECFP cDNA fragments amplified by PCR in which an NheI site was added to the forward primer and an EcoRI site was added to the reverse primer, were digested with NheI/BspEI and subcloned into the pTagRFP-scFv from which the cDNA sequence of TagRFP was removed by NheI/BspEI digestion (pECFP-scFv).

Preparation of Cell-Membrane-Expressed 5D4 Construct

A BspEI site was added to a forward primer and an EcoRI site was added to a reverse primer, and PCR was performed using pTagRFP-5D4 as the template. The cDNA fragments thus acquired were digested with BspEI/EcoRI, and subcloned into a vector obtained by digesting pcDNATagRFP-M13 (251-450 aa)-CAAX (supplied by Tetsuro Ariyoshi of Tokyo University) with BspEI/EcoRI and removing the M13 (251-450 aa) coding region (pTagRFP-5D4-CAAX). The pTagRFP-5D4-CAAX was digested with NheI/BspEI and the TagRFP coding region was removed, and an ECFP-5D4 coding region excised from pECFP-5D4 by NheI/BspEI digestion was subcloned (pECFP-5D4-CAAX).

Preparation of Nuclear Localization Construct

Complementary DNA fragments amplified by PCR in which an SmaI site was added to the forward primer, an SalI site was added to the reverse primer, and pECFP-5D4-CAAX was used as the template, and which were digested by SmaI/SalI, were subcloned into pCMV-SPORT from which an NheI site and EcoRI site were removed in advance and which was digested with SmaI/SalI (pCMV-SPORT6-ECFP-5D4). DNA sequences coding for a GGGS linker and a nuclear localization signal (DPKKKRKVDPKKKRKVDPKKKRKV) were inserted into an EcoRI/NotI site of pCMV-SPORT6-ECFP-5D4 (pCMV-SPORT6-ECFP-5D4-NLS).

Preparation of Endoplasmic-Reticulum Expression Construct

DNA sequences coding for an ER localization signal (GWSCIILFLVATATGAHS) and a GGGAS amino acid linker were prepared by annealing of oligo DNA and inserted into an SmaI/NheI site of pCMV-SPORT6-ECFP-5D4. Additionally, DNA sequences coding for a GGGS linker and an endoplasmic-reticulum localization signal (SEKDEL) were prepared by annealing of oligo DNA and inserted into the EcoRI/NotI site (pCMV-SPORT6-ECFP-5D4-ER).

Preparation of Tubulin Expression Construct

A β-Tubulin-Halo expression construct (TBB-Halo) (S.-n. Uno et al., A spontaneously blinking fluorophore based on intramolecular spirocyclization for live-cell super-resolution imaging. Nat Chem 6, 681-689 (2014)) was digested with SalI/NotI and a HaloTag coding region was removed therefrom, after which a DNA fragment obtained by digesting, with SalI/NotI, a 5D4 coding region to which a SalI/NotI site was added was subcloned by PCR. In the resultant plasmid, sequences coding for a linker in which GGGS occurs twice in succession were added immediately after a tubulin coding region and immediately before a 5D4 coding region of TBB-5D4 by circular PCR. The PCR product was purified and phosphorylated with T4 PNK (TOYOBO), and self-ligation was then performed using a Ligation Kit Version 2 (TAKARA). E. coli HB101 were transformed with the ligation product and cultured overnight on an LB medium plate including 100 μg/mL of ampicillin. A plasmid was acquired from E. coli propagated from a single E. coli colony (pTBB-GGGS4-5D4).

5D4-Actin Expression Construct

A 5D4 cDNA region was acquired from p5D4-actin by NheI/BspEi digestion, and an actin cDNA region was acquired from pmGFP-actin (supplied by Murakoshi Lab, National Institute for Physiological Sciences) (H. Murakoshi, H. Wang, R. Yasuda, Local, persistent activation of Rho GTPases during plasticity of single dendritic spines. Nature 472, 100-104 (2011).) by BspEi/BamHI digestion, and the cDNA regions were subcloned into an NheI/BamHI site of pcDNA3.1(+) (Invitrogen) (p5D4-actin). The p5D4-actin was digested with BspEI/BglII, and linker DNA coding for the amino acids GGGSGGGSGGGSGGGS was formed by annealing of oligo DNA and ligated thereto (p5D4-GGGS4-actin).

Preparation of Lifeact Expression Construct

In order to obtain a cDNA sequence (J. Riedl et al., Lifeact: a versatile marker to visualize F-actin. Nat Methods 5, 605-607 (2008)) coding for a Lifeact peptide, two oligo DNAs were each treated for one hour at 37° C. with T4PNK and phosphorylated, and the two phosphorylated oligo DNAs were then mixed and treated for five minutes at 95° C., and then allowed to cool to room temperature, whereby a double-stranded linker was formed. The linker was subcloned into a vector obtained by NheI digestion of pECFP-5D4 and dephosphorylation by BAP treatment (pLifeact-5D4).

Preparation of 5D4-STIM1 Expression Construct

Complementary DNA fragments amplified by PCR in which an EcoRV site was added to the forward primer, an XbaI site was added to the reverse primer, and pGFP-STIM1 (Y. Wang et al., STIM protein coupling in the activation of Orai channels. Proceedings of the National Academy of Sciences of the United States of America 106, 7391-7396 (2009).) was used as the template were digested by EcoRV/XbaI, and were subcloned into pcDNA3.1(+) (Invitrogen) which was digested with EcoRV/XbaI (p5D4-STIM1).

2. Preparation of Fluorescent Probe in which Fluorescent Dye and DNP are Combined

Methods Used in Organic Synthesis/Compound Identification

All chemical reagents and solvents were obtained from Aldrich, Nacalai Tesque, Tokyo Chemical Industry, Wako Pure Chemical Industries, Thermo Scientific, or Kanto Chemical and used without further purification.

HPLC purification was performed using an HPLC system (JASCO) provided with a pump (PU-2080) and a UV detector (MD-2010), and an Inertsil ODS-3 (5 μm, p 10 mm or i 14 mm×250 mm) (GL Sciences) was used as a reversed-phase column. At this time, samples were filtered by a PTFE filter (0.45 μm) (Millipore) and then purified under a linear gradient condition in which the liquid A (H₂O with 0.1% TFA):liquid B (CH₃CN with 0.1% TFA) ratio changed from 95:5 to 5:95 over 20 minutes. After adding a saturated saline solution to an acquired fraction, a specified substance was acquired by extraction with dichloromethane or ethyl acetate, and drying and concentration by sodium sulfate.

¹H NMR and ¹³C NMR spectra were measured at room temperature using an AVANCE III 400 spectrometer (Bruker). All chemical shifts (δ) are expressed in units of ppm, and tetramethylsilane (0 ppm) or residual solvent (CDCl₃, 7.26 ppm for ¹H, 77.16 ppm for ¹³C; CD₃OD, 3.31 ppm for ¹H, 49.00 ppm for ¹³C; Acetone-d₆, 2.05 ppm for ¹H, 29.84 ppm for ³C; CD₃CN, 1.94 ppm for ¹H, 1.32 ppm for ¹³C; DMSO-d₆, 2.50 ppm for H, 39.52 ppm for ¹³C) was used as an internal standard. Multiplicity of peaks is abbreviated in the following manner: s=singlet, d=doublet, t=triplet, q=quartet, m=multiplet, dd=double doublet, brs=broad singlet. ESI-TOF(electron spray ionization-time-of-flight). Mass spectrometry was performed using a micrOTOF II-TM mass spectrometer (Bruker).

### Abbreviations

DCM: dichloromethane

### DIEPA: N,N-diisopropylethylamine

DMAP: N,N-dimethyl-4-aminopyridine

### DMF: N,N-dimethylformamide

DMSO: dimethyl sulfoxide
DSC: N,N′-Disuccinimidyl carbonate
HATU: 2-(1H-7-azabenzotriazol-1-yl)-1,1,3,3-tetramethyluronium hexafluorophosphate methanaminium
HRMS: high resolution mass spectrometry
TEA: triethylamine
TFA: trifluoroacetic acid
TSTU:O—(N-Succinimidyl)-N,N,N′,N′-tetramethyluronium Tetrafluoroborate

### Synthesis of DNP-Amine (Compound 1)

A DMF solution (16 mL) in which 1-fluoro-2,4-dinitrobenzene (995 mg, 5.34 mmol) was dissolved was slowly dropped at 0° C. into 4 mL of a DMF solution in which 1,2-bis(2-aminoethoxy)ethane (7.92 g, 53.4 mmol) and DIPEA (9.30 mL, 53.4 mmol) were dissolved. The reaction mixture was stirred all night at room temperature, and then concentrated. DCM was added to a residue thereof, and the residue was washed with a 1 M aqueous solution of sodium hydrogen carbonate and dehydrated with sodium sulfate, and was filtered and concentrated. A crude product was purified by silica gel chromatography (elution solvent: ethyl acetate followed by methanol), and DNP-amine (1.20 g, 3.82 mmol) was acquired as a yellow oil (yield: 72%).

¹H NMR (400 MHz, CD₃OD): δ 8.88 (d, 1H, J=2.8 Hz), 8.20 (dd, 1H, J=9.6, 2.8 Hz), 7.14 (d, 1H, J=9.6 Hz), 3.82 (t, 2H, J=5.2 Hz), 3.72-3.63 (m, 6H), 3.53 (t, 2H, J=5.2 Hz), 2.79 (t, 2H, J=5.2 Hz). ¹³C NMR (100 MHz, CD₃OD): δ 149.7, 136.9, 131.3, 130.9, 124.5, 116.0, 73.6, 71.5, 71.3, 69.7, 44.0, 42.1.

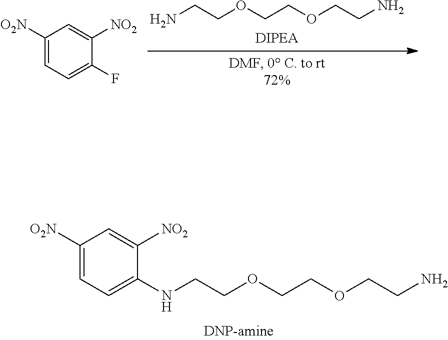 [SMILES: NCCOCCOCCN ; O=[N+]([O-])c1ccc(F)c([N+](=O)[O-])c1 ; NCCOCCOCCNc1ccc([N+](=O)[O-])cc1[N+](=O)[O-]]

Synthesis of DNP

Acetic anhydride (12.0 μL, 0.127 mmol) was dropped into 0.5 mL of an acetonitrile solution in which DNP-amine (40.0 mg, 0.127 mmol) was dissolved. The reaction mixture was stirred all night at room temperature, and then concentrated. A crude product was purified by silica gel chromatography (elution solvent: 3% methanol/ethyl acetate), and DNP (42.5 mg, 0.119 mmol) was acquired as a yellow oil (yield: 94%).

¹H NMR (400 MHz, CDCl₃): δ 9.11 (d, 1H, J=2.8 Hz), 8.88 (br s, 1H), 8.27 (dd, 1H, J=9.6, 2.8 Hz), 6.96 (d, 1H, J=9.6 Hz), 6.27 (br s, 1H), 3.86 (t, 2H, J=5.2 Hz), 3.74-3.57 (m, 8H), 3.47 (q, 2H, J=5.2 Hz), 2.00 (s, 3H). ¹³C NMR (100 MHz, CDCl₃): δ 170.4, 148.4, 136.1, 130.4, 124.3, 114.2, 70.7, 70.2, 68.2, 43.2, 39.4, 23.3. HRMS (ESI⁺) calcd. for [M+H]⁺, 357.14102; found, 357.14158 (Δ0.56 mmu).

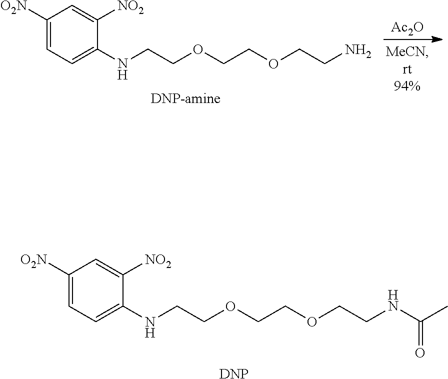 [SMILES: "Ac2O" ; NCCOCCOCCNc1ccc([N+](=O)[O-])cc1[N+](=O)[O-] ; CC(=O)NCCOCCOCCNc1ccc([N+](=O)[O-])cc1[N+](=O)[O-] ; "DNP"]

Synthesis of oNP-Amine

By the same scheme used in the synthesis of DNP-amine, oNP-amine (43.7 mg, 0.162 mmol) was acquired as an orange-colored oil (yield: 70%) using 2-fluoronitrobenzene as a starting material.

¹H NMR (400 MHz, CD₃OD): δ 8.12-8.10 (m, 2H), 7.50-7.46 (m, 1H), 7.03-7.01 (m, 1H), 6.69-6.64 (m, 1H), 3.78 (t, 2H, J=5.2 Hz), 3.68-3.66 (m, 4H), 3.53-3.50 (m, 4H), 2.77 (br s, 2H). ¹³C NMR (100 MHz, CD₃OD): δ 146.7, 137.4, 133.1, 127.5, 116.4, 115.4, 73.6, 71.5, 71.4, 70.1, 43.5, 42.1. HRMS (ESI⁺) calcd. for [M+H]⁺, 270.14483; found, 270.14584 (Δ1.01 mmu).

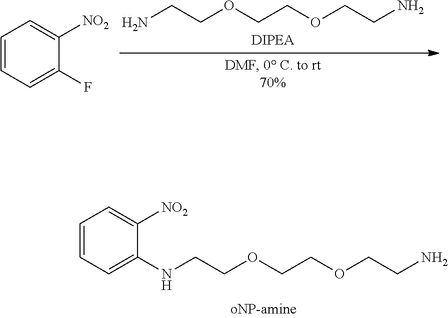 [SMILES: O=[N+]([O-])c1ccccc1F ; NCCOCCOCCN ; NCCOCCOCCNc1ccccc1[N+](=O)[O-]]

Synthesis of oNP

By the same scheme used in the synthesis of DNP, oNP (48.7 mg, 0.156 mmol) was acquired as an orange-colored oil (yield: 84%) using oNP-amine as a starting material.

¹H NMR (400 MHz, DMSO-d₆): δ 8.18 (t, 1H, J=5.2 Hz), 8.07-8.04 (m, 2H), 7.85 (br s, 1H), 7.55-7.51 (m, 1H), 7.07-7.05 (m, 1H), 6.71-6.66 (m, 1H), 3.68 (t, 2H, J=5.6 Hz), 3.60-3.48 (m, 6H), 3.40 (t, 2H, J=6.0 Hz), 3.18 (q, 2H, J=5.6 Hz), 1.79 (s, 3H). ¹³C NMR (100 MHz, CD₃OD): δ 169.3, 145.2, 136.6, 131.0, 126.2, 115.4, 114.6, 69.7, 69.6, 69.2, 68.4, 42.1, 38.6, 22.5. HRMS (ESI⁺) calcd. for [M+H]⁺, 312.15540; found, 312.15452 (Δ−0.88 mmu).

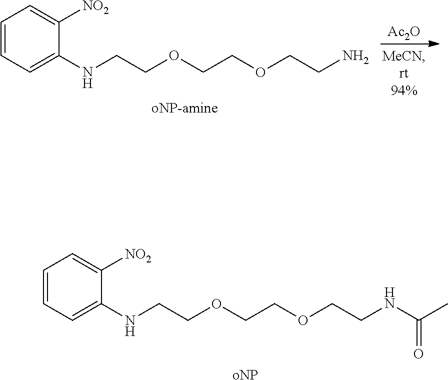 [SMILES: "Ac2O" ; NCCOCCOCCNc1ccccc1[N+](=O)[O-] ; CC(=O)NCCOCCOCCNc1ccccc1[N+](=O)[O-] ; "oNP"]

Synthesis of pNP-Amine

By the same scheme used in the synthesis of DNP-amine, pNP-amine (47.0 mg, 0.174 mmol) was acquired as a yellow solid (yield: 73%) using 4-fluoronitrobenzene as a starting material.

¹H NMR (400 MHz, CD₃OD): δ 8.04-8.01 (m, 2H), 6.66-6.64 (m, 2H), 3.72-3.69 (m, 8H), 3.41 (t, 2H, J=5.2 Hz), 3.11 (t, 2H, J=5.2 Hz). ¹³C NMR (100 MHz, CD₃OD): δ 156.0, 138.1, 127.3, 111.9, 71.4, 71.3, 70.4, 67.9, 43.8, 40.6. HRMS (ESI⁺) calcd. for [M+H]⁺, 270.14538; found, 270.14517 (Δ−0.21 mmu).

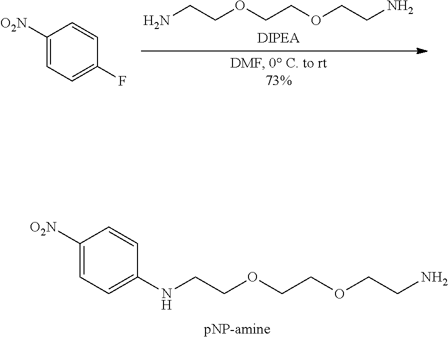 [SMILES: NCCOCCOCCN ; O=[N+]([O-])c1ccc(F)cc1 ; NCCOCCOCCNc1ccc([N+](=O)[O-])cc1]

Synthesis of pNP

By the same scheme used in the synthesis of DNP, pNP (46.8 mg, 0.150 mmol) was acquired as a yellow oil (yield: 81%) using pNP-amine as a starting material.

¹H NMR (400 MHz, DMSO-d₆): δ 7.93 (m, 2H), 7.87 (br s, 1H), 7.31 (t, 1H, J=5.6 Hz), 6.67 (m, 2H), 3.59-3.51 (m, 6H), 3.40-3.31 (m, 4H), 3.17 (q, 2H, J=5.6 Hz), 1.79 (s, 3H). ¹³C NMR (100 MHz, DMSO-d₆): δ 169.3, 154.6, 135.7, 126.2, 110.9 (br s), 69.7, 69.6, 69.2, 68.7, 42.4, 38.6, 22.6. HRMS (ESI⁺) calcd. for [M+H]⁺, 312.15540; found, 312.15644 (Δ1.04 mmu).

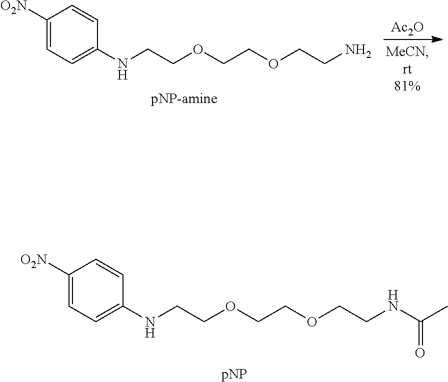 [SMILES: "Ac2O" ; NCCOCCOCCNc1ccc([N+](=O)[O-])cc1 ; CC(=O)NCCOCCOCCNc1ccc([N+](=O)[O-])cc1 ; "pNP"]

Synthesis of DNP2-Amine

By the same scheme used in the synthesis of DNP-amine, DNP2-amine (254 mg, 0.939 mmol) was acquired as a yellow solid (yield: 78%) using 2,2′-oxybis(ethylamine) as a starting material. ¹H NMR (400 MHz, CD₃OD): δ 8.95 (d, 1H, J=2.8 Hz), 8.24 (dd, 1H, J=9.6, 2.8 Hz), 7.18 (d, 1H, J=9.6 Hz), 3.79 (t, 2H, J=5.2 Hz), 3.67 (t, 2H, J=5.2 Hz), 3.57 (t, 5.2 Hz, 2H), 2.82 (t, 2H, J=5.2 Hz). ¹³C NMR (100 MHz, CD₃OD): δ 149.8, 137.0, 131.5, 130.9, 124.9, 116.0, 73.6, 69.7, 44.0, 42.2. HRMS (ESI⁻) calcd for [M−H]⁻, 269.08914; found, 269.09115 (Δ 2.01 mmu).

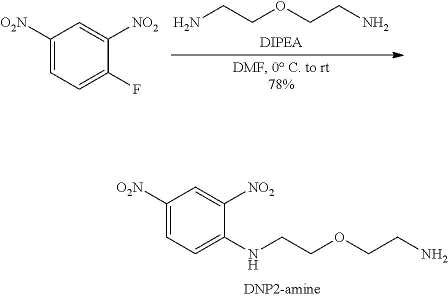 [SMILES: NCCOCCN ; O=[N+]([O-])c1ccc(F)c([N+](=O)[O-])c1 ; NCCOCCNc1ccc([N+](=O)[O-])cc1[N+](=O)[O-]]

Synthesis of DNP4-Amine

By the same scheme used in the synthesis of DNP-amine, DNP4-amine (197 mg, 0.549 mmol) was acquired as a yellow oil (yield: 83%) using 1,11-diamino-3,6,9-trioxaundecane as a starting material.

¹H NMR (400 MHz, CD₃OD): δ 8.96 (d, 1H, J=2.4 Hz), 8.24 (dd, 1H, J=9.6, 2.8 Hz), 7.19 (d, 1H, J=9.6 Hz), 3.81 (t, 2H, J=5.2 Hz), 3.69-3.60 (m, 10H), 3.51 (t, 2H, J=5.2 Hz), 2.77 (t, 2H, J=5.2 Hz). ¹³C NMR (100 MHz, CD₃OD): δ 149.8, 137.0, 131.5, 131.0, 124.6, 116.1, 73.4, 71.6, 71.58, 71.3, 69.9, 44.1, 42.

1. HRMS (ESI⁻) calcd for [M−H]⁻, 357.14157; found, 357.14466 (Δ 3.09 mmu).

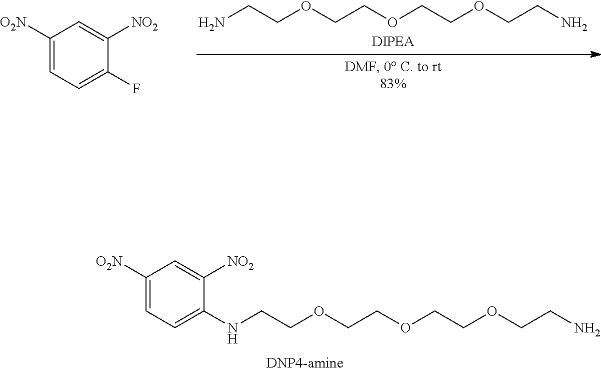 [SMILES: O=[N+]([O-])c1ccc(F)c([N+](=O)[O-])c1 ; NCCOCCOCCOCCN ; NCCOCCOCCOCCNc1ccc([N+](=O)[O-])cc1[N+](=O)[O-]]

Synthesis of DNP-NHS

A DMF solution (32 μL) of 1 M DNP-amine (10 mg, 0.032 mmol equivalent) was dropped at 0° C. into 0.5 mL of a DMF solution in which disuccinimidyl suberate (120 mg, 0.032 mmol) and DIPEA (11 μL, 0.064 mmol) were dissolved, and the reaction mixture was then stirred for two hours at room temperature. A coarse product was purified by HPLC, and DNP-NHS (7.9 mg, 0.014 mmol) was acquired as a yellow solid (yield: 44%).

¹H NMR (400 MHz, CDCl₃): δ 9.15 (d, 1H, J=2.8 Hz), 8.88 (br s, 1H), 8.29 (dd, 1H, J=9.6, 2.8 Hz), 6.94 (d, 1H, J=9.6 Hz), 6.34 (br s, 1H), 3.84 (t, 2H, J=5.2 Hz), 3.73-3.66 (m, 4H), 3.51-3.47 (m, 4H), 2.84 (br s, 4H), 2.60 (t, 2H, J=7.6 Hz), 2.24 (t, 2H, J=7.6 Hz), 1.78-1.62 (m, 4H), 1.43-1.33 (m, 4H). ¹³C NMR (100 MHz, CDCl₃): δ 174.2, 169.4, 168.7, 148.4, 136.4, 130.6, 124.5, 114.1, 70.8, 70.3, 70.2, 68.3, 43.2, 39.6, 36.3, 31.0, 28.6, 28.4, 25.7, 25.5, 24.5.

HRMS (ESI⁺) calcd. for [M+Na]⁺, 590.20688; found, 590.20688 (Δ 0.01 mmu).

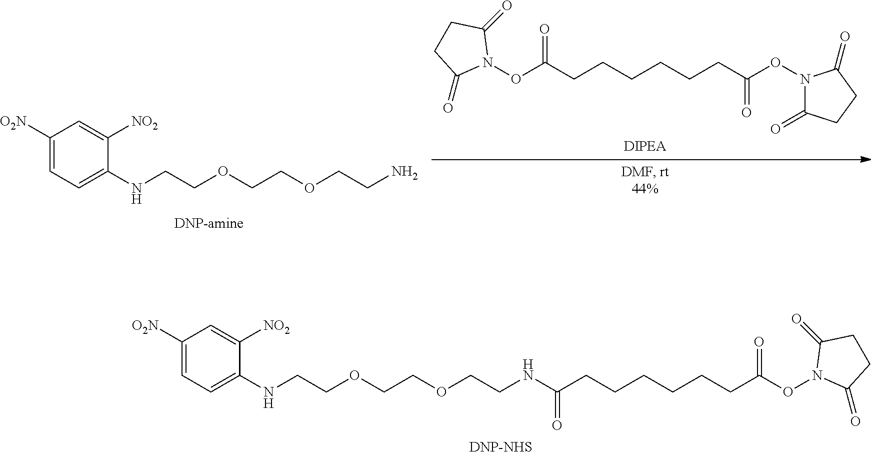

### Synthesis Example 1

### Synthesis of 6DCF-DNP (2-(2,7-dichloro-6-hydroxy-3-oxo-3H-xanthene-9-yl)-4-((2-(2-(2-((2,4-dinitrophenyl)amino)ethoxy)ethoxy)ethyl)carbamoyl)benzoate)

A reaction mixture in which 3′,6′-diacetyl-2′,7′-dichloro-6-carbonylfluoroscein pyridinium salt (Woodroofe, C. C., Masalha, R., Barnes, K. R., Frederickson, C. J. & Lippard, S. J. Membrane-permeable and-impermeable sensors of the Zinpyr family and their application to imaging of hippocampal zinc invivo. Chem. Biol. 11, 1659-1666 (2004).) (26 mg, 0.050 mmol), DNP-amine (19 mg, 0.059 mmol), HATU (23 mg, 0.059 mmol), and DIPEA (43 μL, 0.25 mmol) were dissolved in 3 mL of acetonitrile was stirred for 30 minutes at room temperature while shielded from light. The reaction mixture was concentrated, after which a coarse product was purified by silica gel chromatography (elution solvent: ethyl acetate/hexane=3/1 followed by 10% methanol/DCM), and an intermediate (24 mg, 0.029 mmol) was acquired. The acquired intermediate was dissolved in THF/water (3 mL/1 mL), 170 μL of a 1 M NaOH aqueous solution was added thereto, and the reaction solution was stirred for 30 minutes at room temperature while shielded from light. Water was added to the reaction solution, and the solution was washed with ethyl acetate, after which 300 μL of 1 M hydrochloric acid was added to a water layer, and extraction was performed with ethyl acetate. An acquired organic layer was dehydrated with sodium sulfate, and then filtered and concentrated. A coarse product was purified by HPLC, and 6DCF-DNP (15 mg, 0.020 mmol) was acquired as a yellow solid (yield of the two reactions: 40%).

¹H NMR (400 MHz, DMSO-d₆): δ 8.82 (d, 2H, J=2.8 Hz), 8.79 (t, 1H, J=5.6 Hz), 8.73 (t, 1H, J=5.6 Hz), 8.21 (dd, 1H, J=9.6, 2.4 Hz), 8.14 (dd, 1H, J=8.0, 1.2 Hz), 8.06 (d, 1H, J=8.0 Hz), 7.19 (d, 1H, J=9.6 Hz), 6.91 (s, 2H), 6.73 (s, 2H), 3.63-3.48 (m, 12H). ¹³C NMR (100 MHz, DMSO-d₆): δ 167.7, 164.7, 155.3, 151.8, 150.0, 148.3, 140.9, 134.9, 129.8, 129.6, 128.4, 127.9, 125.3, 123.5, 122.2, 116.4, 115.5, 110.0, 103.6, 81.7, 69.7, 69.5, 68.7, 68.2, 42.6. HRMS (ESI⁺) calcd. for [M+H]⁺, 741.09970; found, 741.10088 (Δ1.18

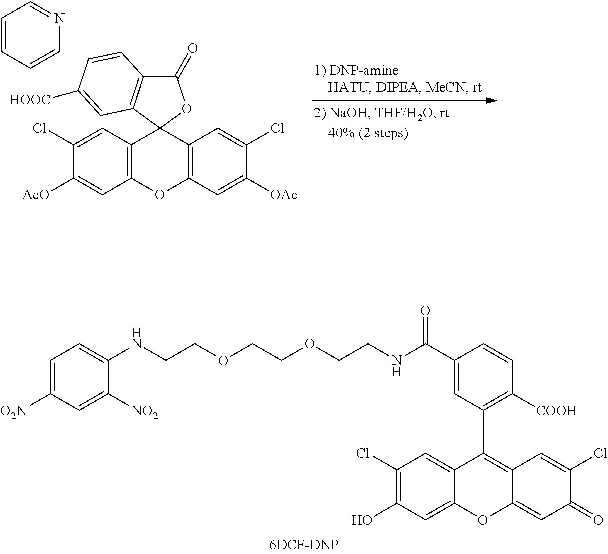

### Synthesis Example 2

### Synthesis of 6DCF-oNP (2-(2,7-dichloro-6-hydroxy-3-oxo-3H-xanthene-9-yl)-4-((2-(2-(2-((2-nitrophenyl)amino)ethoxy)ethoxy)ethyl)carbamoyl)benzoate)

By the same scheme used in the synthesis of 6DCF-DNP, 6DCF-oNP (12 mg, 0.017 mmol) was acquired as a yellow solid (yield of the two reactions: 33%) using oNP-amine as a starting material.

¹H NMR (400 MHz, DMSO-d₆): δ 11.12 (s, 2H), 8.74 (t, 1H, J=4.8 Hz), 8.14-8.02 (m, 4H), 7.69 (s, 1H), 7.53-7.49 (m, 1H), 7.02 (d, 1H, J=8.8 Hz), 6.91 (s, 2H), 6.74 (s, 2H), 6.69-6.65 (m, 1H), 3.62-3.43 (m, 12H). HRMS (ESI⁺) calcd. for [M+H]⁺, 696.11463; found, 696.11539 (Δ0.76 mmu).

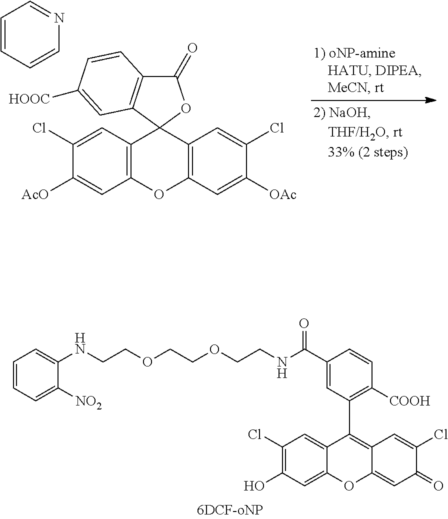

### Synthesis Example 3

### Synthesis of 6DCF-pNP (2-(2,7-dichloro-6-hydroxy-3-oxo-3H-xanthene-9-yl)-4-((2-(2-(2-((4-nitrophenyl)amino)ethoxy)ethoxy)ethyl)carbamoyl)benzoate)

By the same scheme used in the synthesis of 6DCF-DNP, 6DCF-pNP (7.7 mg, 0.011 mmol) was acquired as a yellow solid (yield of the two reactions: 29%) using pNP-amine as a starting material.

¹H NMR (400 MHz, CD₃OD): δ 8.16 (d, 1H, J=1.6 Hz), 8.14 (d, 1H, J=1.6 Hz), 8.10-7.95 (m, 2H), 7.65 (s, 1H), 6.82 (s, 2H), 6.66 (s, 1H), 6.55 (d, 2H, J=9.6 Hz), 3.61-3.52 (m, 10H), 3.26-3.24 (m, 2H). HRMS (ESI⁺) calcd. for [M+H]⁺, 696.11463; found, 696.11560 (Δ0.97 mmu).

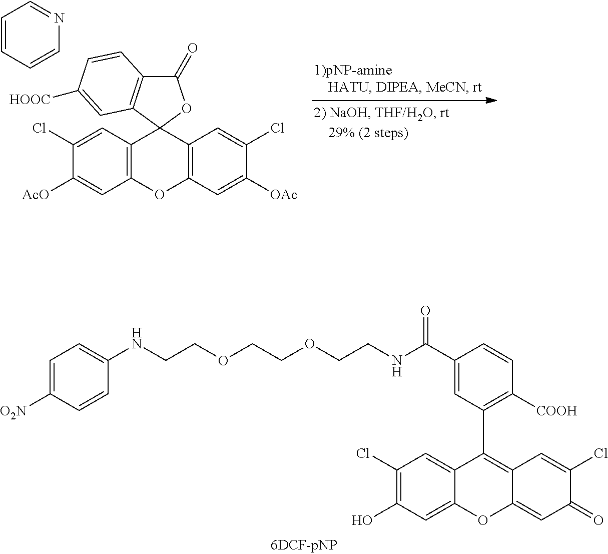

### Synthesis Example 4

### Synthesis of 6DCF-DNP2 (2-(2,7-dichloro-6-hydroxy-3-oxo-3H-xanthene-9-yl)-4-((2-(2-((2,4-dinitrophenyl)amino)ethoxy)ethyl)carbamoyl)benzoate)

By the same scheme used in the synthesis of 6DCF-DNP, 6DCF-DNP2 (4.7 mg, 0.0067 mmol) was acquired as an orange solid (yield of the two reactions: 8.9%) using DNP2-amine as a starting material.

¹H NMR (400 MHz, DMSO-d₆): δ 8.94 (d, 1H, J=2.8 Hz), 8.82 (br s, 1H), 8.25-8.19 (m, 2H), 8.07-8.05 (m, 2H), 7.80 (br s, 1H), 7.27 (d, 1H, J=9.6 Hz), 7.00 (s, 2H), 6.86 (s, 2H), 3.81 (t, 2H, J=5.2 Hz), 3.71 (q, 2H, J=5.2 Hz), 3.67 (t, 2H, J=5.6 Hz), 3.56 (q, 2H, J=5.6 Hz). HRMS (ESI⁺) calcd. for [M+H]⁺, 697.07349; found, 697.07700 (Δ 3.51 mmu).

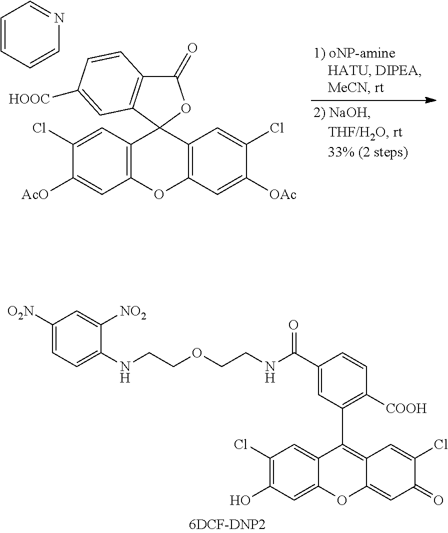

### Synthesis Example 5

### Synthesis of 6DCF-DNP4 (2-(2,7-dichloro-6-hydroxy-3-oxo-3H-xanthene-9-yl)-4-((2-(2-(2-(2-((2,4-dinitrophenyl)amino)ethoxy)ethyl)carba moyl)benzoate)

By the same scheme used in the synthesis of 6DCF-DNP, 6DCF-DNP4 (3.0 mg, 0.0038 mmol) was acquired as an orange solid (yield of the two reactions: 5.0%) using DNP4-amine as a starting material.

¹H NMR (400 MHz, DMSO-d₆): δ 8.96 (d, 1H, J=2.8 Hz), 8.83 (br s, 1H), 8.29-8.25 (m, 1H), 8.22 (dd, 1H, J=1.2, 8.0 Hz), 8.07 (dd, 1H, J=0.8, 8.0 Hz), 7.97 (t, 1H, J=5.6 Hz), 7.80 (br s, 1H), 7.26 (d, 1H, J=9.6 Hz), 7.00 (s, 2H), 6.86 (s, 2H), 3.78 (t, 2H, J=5.2 Hz), 3.69 (q, 2H, J=5.2 Hz), 3.59-3.46 (m, 12H). HRMS (ESI⁺) calcd. for [M+H]⁺, 785.12592; found, 785.12616(Δ 0.24 mmu).

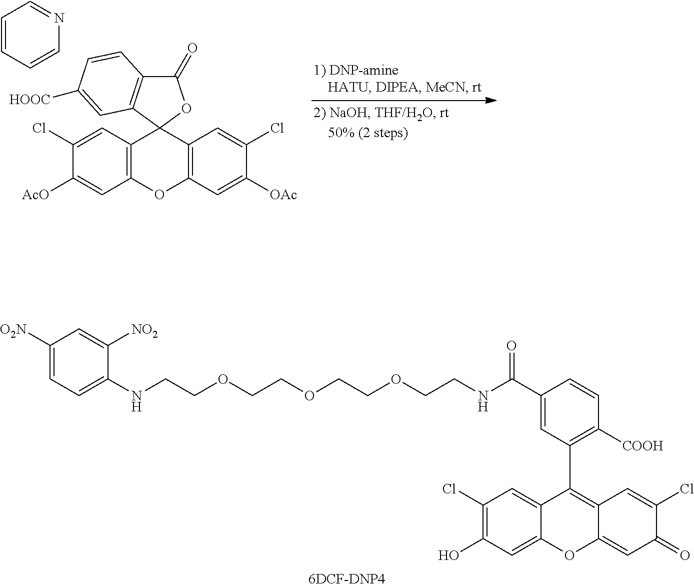

### Synthesis Example 6

### Synthesis of DCF-DNP (2-(2,7-dichloro-6-hydroxy-3-oxo-3H-xanthene-9-yl)-N-(2-((2-(2-(2-((2,4-dinitrophenyl)amino)ethoxy)ethoxy)ethyl)amino)-2-oxo ethyl)-N-methylbenzamide)

In 1 mL of DMF were dissolved 2′,7′-dichlorofluorescein (51 mg, 0.13 mmol), tert-butylsarcosinate hydrochloride (28 mg, 0.15 mmol), HATU (58 mg, 0.15 mmol), and DIPEA (111 μL, 0.64 mmol), and the reaction mixture was stirred for one day at room temperature while shielded from light. A coarse product was purified by HPLC, and a tert-butyl ester intermediate was acquired. After 1 mL of TFA was dropped into 5 mL of DCM in which the intermediate and triethyl silane (56 μL) were dissolved, the reaction mixture was stirred all night at room temperature while shielded from light. A coarse product was purified by HPLC, and an intermediate having a carboxylic acid was acquired as an orange solid. A reaction mixture in which the acquired intermediate, compound 1 (46 mg, 0.15 mmol), HATU (56 mg, 0.15 mmol), and DIPEA (106 μL, 0.61 mmol) were dissolved in 1 mL of DMF was stirred for two hours at room temperature while shielded from light. A crude product was purified by HPLC, and DCF-DNP (29 mg, 0.038 mmol) was acquired as an orange solid (yield of the three reactions: 30%).

HRMS (ESI⁻) calcd. for [M−H]⁻, 766.13244; found, 766.13345 (Δ1.01 mmu).

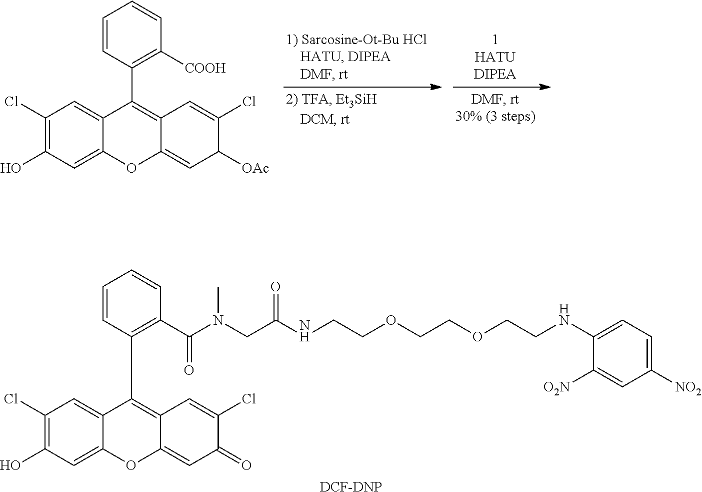 [SMILES: CC(=O)OC1C=C2Oc3cc(O)c(Cl)cc3C(c3ccccc3C(=O)O)=C2C=C1Cl ; CN(CC(=O)NCCOCCOCCNc1ccc([N+](=O)[O-])cc1[N+](=O)[O-])C(=O)c1ccccc1-c1c2cc(Cl)c(=O)cc-2oc2cc(O)c(Cl)cc12 ; "DCF-DNP"]

### Synthesis Example 7

### Synthesis of R110-DNP (6-amino-9-(2-((2-(2-((2-((((2-(2,4-dinitrophenyl)amino) ethoxy) ethoxy)ethyl)amino)-2-oxoethyl) (methyl)carbamoyl)phenyl)-3H-xanthene-3-iminium)

By the same scheme used in the synthesis of DCF-DNP, R110-DNP (4.8 mg, 0.0065 mmol) was acquired as an orange solid (yield of the three reactions: 15%) using rhodamine 110 chloride as a starting material.

HRMS (ESI⁺) calcd. for [M-Cl]⁺, 698.25690; found, 698.25345 (Δ-3.45 mmu).

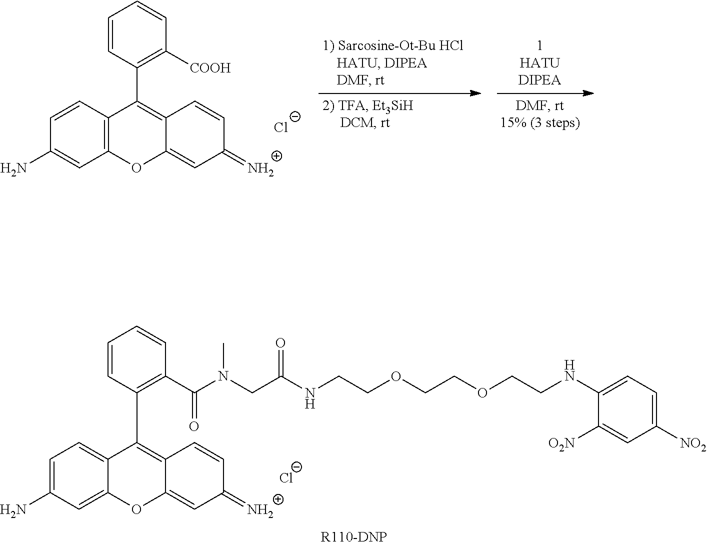 [SMILES: Nc1ccc2c(-c3ccccc3C(=O)O)c3ccc(=[NH2+])cc-3oc2c1 ; [Cl-] ; CN(CC(=O)NCCOCCOCCNc1ccc([N+](=O)[O-])cc1[N+](=O)[O-])C(=O)c1ccccc1-c1c2ccc(=[NH2+])cc-2oc2cc(N)ccc12 ; [Cl-] ; "R110-DNP"]

### Synthesis Example 8

### Synthesis of 50G-DNP (2-(2,7-difluoro-6-hydroxy-3-oxo-3H-xanthene-9-yl)-5-((2-(2-(2-((2,4-dinitrophenyl)amino)ethoxy)ethoxy)ethyl)carbamoyl)benzoate)

By the same scheme used in the synthesis of DCF-DNP, 50G-DNP (2.9 mg, 0.0041 mmol) was acquired as an orange solid (yield: 34%) using 5-carboxy-2′,7′-difluorofluorescein (W.-C. Sun, K. R. Gee, D. H. Klaubert, R. P. Haugland, Synthesis of Fluorinated Fluoresceins. The Journal of Organic Chemistry 62, 6469-6475 (1997)) as a starting material.

¹H NMR (400 MHz, DMSO-d₆): δ 8.86-8.82 (m, 3H), 8.44 (m, 1H), 8.25-8.20 (m, 2H), 7.42 (d, J=8.0 Hz, 1H), 7.24 (d, J=9.6 Hz, 1H), 6.90 (s, 2H), 6.73 (s, 2H), 3.71 (t, J=5.6 Hz, 2H), 3.61-3.45 (m, 10H). ¹³C NMR (100 MHz, DMSO-d₆): δ 167.7, 164.7, 155.3, 153.6, 150.1, 148.4, 136.5, 134.9, 129.9, 129.7, 128.7, 128.4, 127.9, 126.4, 124.1, 123.9, 123.6, 116.4, 115.6, 110.0, 103.7, 69.8, 69.6, 68.8, 68.3, 48.6, 42.7. HRMS (ESI⁻) calcd. for [M−H]⁻, 707.14425; found, 707.14452 (Δ0.27 mmu).

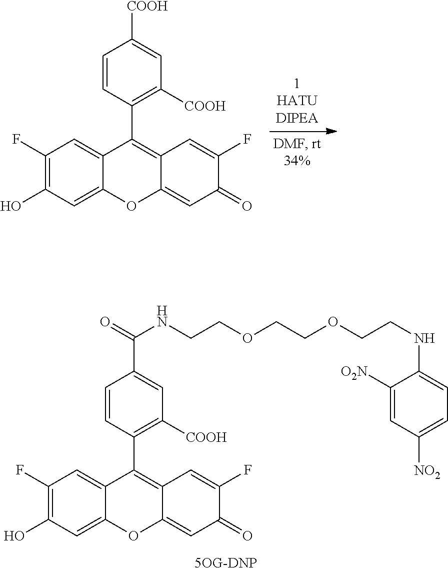

### Synthesis Example 9

### Synthesis of 6SiR-DNP (2-(7-(dimethylamino)-3-(dimethylimino)-5,5-dimethyl-3,5-dihydrodibenzo[b,e]silin-10-yl)-4-((2-(2-(2-((2,4-dinitrophenyl)amino)ethoxy)ethoxy)ethyl)carbamoyl)benzoate)

A reaction mixture in which SiR-carboxyl (G. Lukinavicius et al., A near-infrared fluorophore for live-cell super-resolution microscopy of cellular proteins. Nat Chem 5, 132-139 (2013)) (5.9 mg, 0.012 mmol), DSC (13 mg, 0.050 mmol), TEA (10 μL, 0.075 mmol), and DMAP (0.2 mg, 0.001 mmol) were dissolved in 1 mL of DMF was stirred all night at room temperature while shielded from light. A coarse product was purified by HPLC, and an intermediate was acquired as a blue solid. A reaction mixture in which the intermediate, compound1 (DNP-amine) (3.9 mg, 0.012 mmol), and DIPEA (5.3 μL) were dissolved in 0.5 mL of DMF was stirred for one hour at room temperature while shielded from light. A coarse product was purified by HPLC, and 6SiR-DNP (3.3 mg, 0.0043 mmol) was acquired as a green solid (yield of the two reactions: 34%).

¹H NMR (400 MHz, Acetone-d₆): δ 8.92 (d, J=2.8 Hz, 1H), 8.79 (br s, 1H), 8.23 (dd, J=2.8, 9.6 Hz, 1H), 8.10-8.08 (m, 1H), 7.97-7.95 (m, 2H), 7.77 (m, 1H), 7.15 (d, J=9.6 Hz, 1H), 7.11 (d, J=2.8 Hz, 2H), 6.75 (d, J=8.8 Hz, 2H), 6.64 (dd, J=2.8, 8.8 Hz, 2H), 3.75 (t, J=5.2 Hz, 2H), 2.96 (s, 12H), 0.69 (s, 3H), 0.55 (s, 3H). ¹³C NMR (100 MHz, Acetone-d₆): δ 170.0, 166.2, 156.0, 150.5, 149.5, 141.2, 138.3, 137.4, 136.6, 132.4, 130.7, 129.3, 129.0, 128.9, 126.2, 124.4, 124.0, 117.6, 116.0, 114.5, 71.1, 70.9, 70.1, 69.3, 43.8, 40.6, 40.3, 0.4, −1.1. HRMS (ESI⁺) calcd. for [M+H]⁺, 769.30118; found, 769.30220 (Δ1.02 mmu).

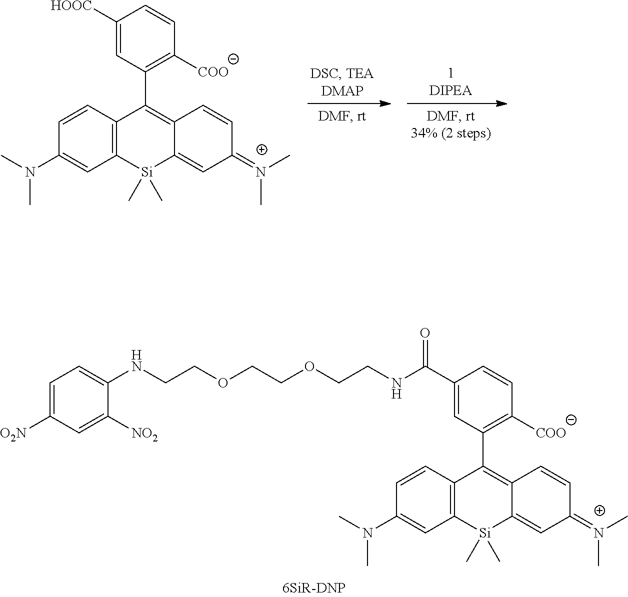

### Synthesis Example 10

### Synthesis of 5DCF-DNP (2-(2,7-dichloro-6-hydroxy-3-oxo-3H-xanthene-9-yl)-5-((2-(2-(2-((2,4-dinitrophenyl)amino)ethoxy)ethoxy)ethyl)carbamoyl)benzoate)

By the same scheme used in the synthesis of 6DCF-DNP, 5DCF-DNP (17 mg, 0.023 mmol) was acquired as a yellow solid (yield of the two reactions: 24%) using 3′,6′-diacetyl-2′,7′-dichloro-5-carboxyfluorescein (Woodroofe, C. C., Masalha, R., Barnes, K. R., Frederickson, C. J. & Lippard, S. J. Membrane-permeable and-impermeable sensors of the Zinpyr family and their application to imaging of hippocampal zinc in vivo. Chem. Biol. 11, 1659-1666 (2004).) as a starting material.

¹H NMR (400 MHz, Acetone-d₆): δ 8.96 (d, 1H, J=2.8 Hz), 8.87 (br s, 1H), 8.44-8.43 (m, 1H), 8.32-8.30 (m, 1H), 8.26-8.23 (m, 1H), 8.12-8.09 (m, 1H), 7.46-7.44 (m, 1H), 7.27-7.24 (m, 1H), 6.98 (s, 2H), 6.84 (s, 2H), 3.89-3.86 (m, 2H), 3.73-3.68 (m, 8H), 3.66-3.62 (m, 2H). ¹³C NMR (100 MHz, DMSO-d₆): δ 167.8, 164.7, 155.3, 153.6, 150.1, 148.4, 136.5, 134.9, 129.9, 129.7, 128.7, 128.4, 127.9, 126.4, 124.1, 123.9, 123.6, 116.4, 115.6, 110.0, 103.7, 69.8, 69.6, 68.8, 68.3, 48.6, 42.7. HRMS (ESI⁺) calcd for [M+H]⁺, 741.10025; found, 741.09788 (Δ-2.37 mmu).

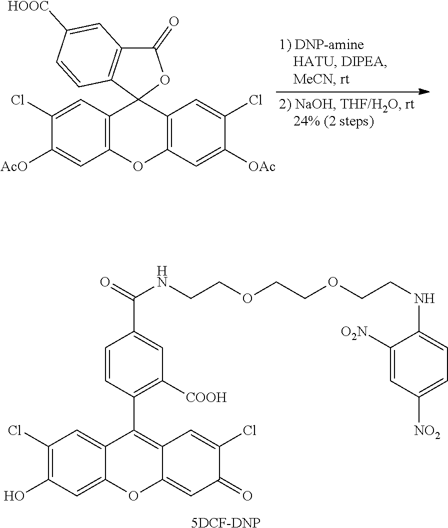

### Synthesis Example 11

### Synthesis of 60G-DNP, diAc (6-((2-(2-(2-((2,4-dinitrophenyl)amino)ethoxy)ethoxy)ethyl)carbamoyl)-2′,7′-difluoro-3-oxo-3H-spiro[isobenzofuran-1,9′-xanthene]-3′,6′-diyl diacetate)

A reaction mixture in which 6-carboxy-2′,7′-difluorofluorescein diacetate, pyridinium salt (Sun, W. C., Gee, K. R., Klaubert, D. H. & Haugland, R. P. Synthesis of fluorinated fluoresceins. J. Org. Chem. 62, 6469-6475 (1997).) (31 mg, 0.063 mmol), DNP-amine (24 mg, 0.075 mmol), HATU (29 mg, 0.075 mmol), and DIPEA (55 μL, 0.31 mmol) were dissolved in 3 mL of acetonitrile was stirred for one hour at room temperature while shielded from light. The reaction mixture was concentrated, after which a coarse product was purified by silica gel chromatography (elution solvent: ethyl acetate/hexane=3/1), and 60C-DNP, diAc (28 mg, 0.035 mmol) was acquired as a yellow solid (yield: 59%).

¹H NMR (400 MHz, DMSO-d₆): δ 8.82 (d, 2H, J=2.8 Hz), 8.80 (t, 1H, J=5.6 Hz), 8.72 (t, 1H, J=5.6 Hz), 8.22 (dd, 1H, J=9.6, 2.8 Hz), 8.18 (d, 1H, J=8.0 Hz), 8.10 (d, 1H, J=8.0 Hz), 7.81 (s, 1H), 7.51 (d, 2H, ⁴J_{HF}=6.4 Hz), 7.21 (d, 1H, J=9.6 Hz), 7.02 (d, 2H, ³J_{HF}=10.4 Hz), 3.63-3.47 (m, 12H), 2.34 (s, 6H). HRMS (ESI⁺) calcd. for [M+H]⁺, 793.17993; found, 793.17871 (Δ-1.22 mmu).

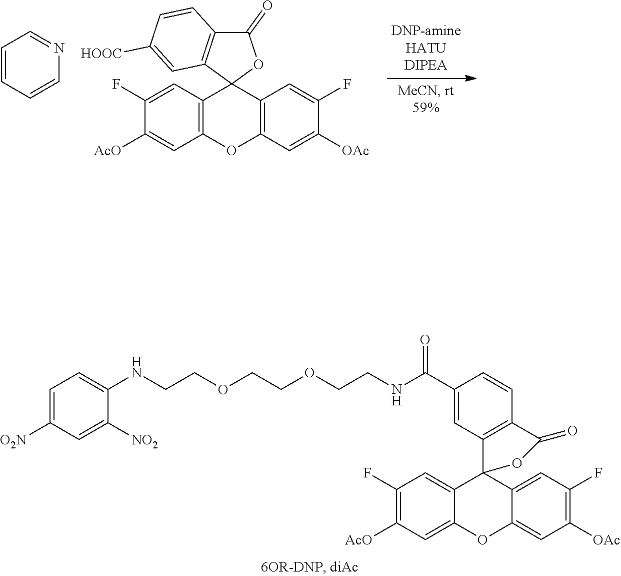 [SMILES: c1ccncc1 ; CC(=O)Oc1cc2c(cc1F)C1(OC(=O)c3ccc(C(=O)O)cc31)c1cc(F)c(OC(C)=O)cc1O2 ; CC(=O)Oc1cc2c(cc1F)C1(OC(=O)c3ccc(C(=O)NCCOCCOCCNc4ccc([N+](=O)[O-])cc4[N+](=O)[O-])cc31)c1cc(F)c(OC(C)=O)cc1O2]

### Synthesis of 60G-DNP (2-(2,7-difluoro-6-hydroxy-3-oxo-3H-xanthene-9-yl)-4-((2-(2-(2-(2,4-dinitrophenyl)amino)ethoxy)ethoxy)ethyl)carbamoyl)benzoate)

60G-DNP, diAc (28 mg, 0.035 mmol) was dissolved in THF/water (2 mL/1 mL), 320 μL of 1 M NaOH aqueous solution was added thereto, and the reaction solution was stirred for 30 minutes at room temperature while shielded from light. Water was added to the reaction solution, and the solution was washed with ethyl acetate, after which 400 μL of 1 M hydrochloric acid was added to a water layer, and extraction was performed with ethyl acetate. An acquired organic layer was dehydrated with sodium sulfate, and then filtered and concentrated. A coarse product was purified by HPLC, and 60G-DNP (17 mg, 0.024 mmol) was acquired as a yellow solid (yield: 68%).

¹H NMR (400 MHz, CD₃OD): δ 8.88 (d, 1H, J=2.8 Hz), 8.21 (dd, 1H, J=9.6, 2.8 Hz), 8.10-8.09 (m, 2H), 7.71 (m, 1H), 7.08 (d, 1H, J=9.6 Hz), 6.66-6.63 (m, 4H), 3.70 (t, 2H, J=5.6 Hz), 3.66-3.63 (m, 6H), 3.57 (t, 2H, J=5.2 Hz), 3.51 (t, 2H, J=5.2 Hz). HRMS (ESI⁺) calcd. for [M+H]⁺, 708.15153; found, 708.15223 (Δ0.70 mmu).

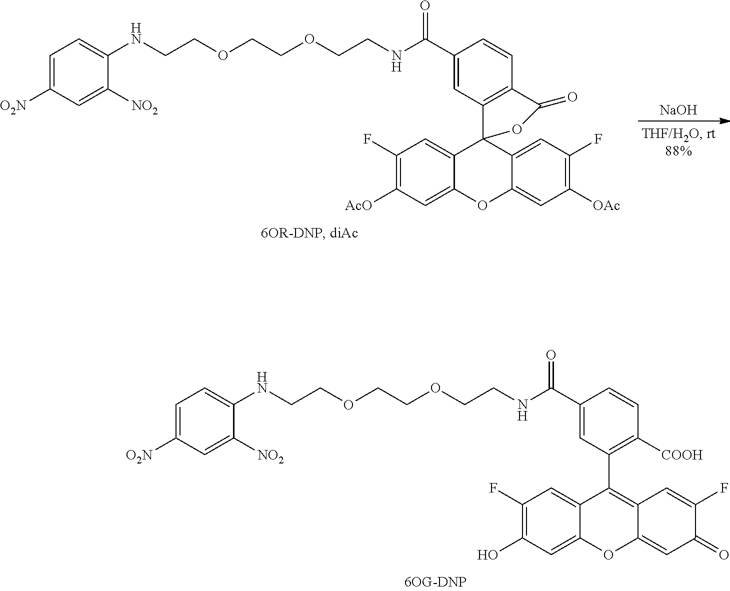 [SMILES: CC(=O)Oc1cc2c(cc1F)C1(OC(=O)c3ccc(C(=O)NCCOCCOCCNc4ccc([N+](=O)[O-])cc4[N+](=O)[O-])cc31)c1cc(F)c(OC(C)=O)cc1O2 ; "NaOH" ; O=C(NCCOCCOCCNc1ccc([N+](=O)[O-])cc1[N+](=O)[O-])c1ccc(C(=O)O)c(-c2c3cc(F)c(=O)cc-3oc3cc(O)c(F)cc23)c1]

### Synthesis Example 12

### 6JF549-DNP (2-(3-(azetidine-1-ium-1-ylidene)-6-(azetidine-1-yl)-3H-xanthene-9-yl)-4-((2-(2-(2-((2,4-dinitrophenyl)amino) ethoxy)ethoxy)ethyl)carbamoyl)benzoate)

By the same scheme used in the synthesis of 6SiR-DNP, 6JF549-DNP (12 mg, 0.016 mmol) was acquired as a violet solid (yield: 30%) using 6-carboxy-JF₅₄₉ (Grimm, J. B. et al. A general method to improve fluorophores for live-cell and single-molecule microscopy. Nat. Methods 12, 244-250 (2015).) as a starting material.

¹H NMR (400 MHz, CD₃OD): δ 8.82 (d, 1H, J=2.4 Hz), 8.32 (d, 1H, J=8.0 Hz), 8.20-8.16 (m, 2H), 7.78 (s, 1H), 7.10 (d, 1H, J=9.6 Hz), 7.00 (d, 2H, J=9.2 Hz), 6.53 (dd, 2H, J=9.2, 1.6 Hz), 6.40 (d, 2H, J=1.6 Hz), 4.26 (t, 8H, J=7.6 Hz), 3.70-3.59 (m, 10H), 3.48 (t, 2H, J=5.2 Hz), 2.55 (quint, 4H, J=7.6 Hz). ¹³C NMR (100 MHz, CD₃OD): δ 168.0, 167.2, 160.0, 158.6, 157.9, 149.7, 139.5, 137.0, 135.5, 134.8, 132.8, 132.2, 131.2, 131.0, 130.1, 124.6, 116.1, 114.7, 113.5, 95.1, 71.5, 71.2, 70.5, 69.7, 52.8, 44.0, 41.2, 30.7, 16.8. HRMS (ESI⁺) calcd for [M+H]⁺, 751.27277; found, 751.27408 (Δ1.31 mmu).

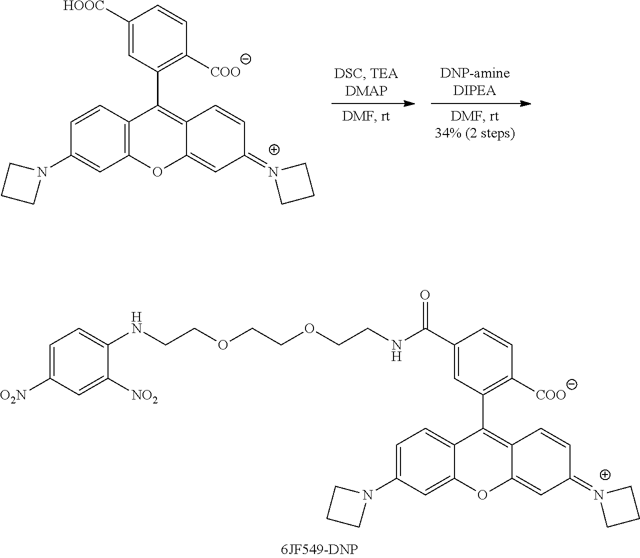

### Synthesis Example 13

### Synthesis of 6JF646, NHS

A reaction mixture in which 6-carboxy-JF₆₄₆ (Grimm, J. B. et al. A general method to improve fluorophores for live-cell and single-molecule microscopy. Nat. Methods 12, 244-250 (2015).) (7.7. mg, 0.015 mmol), DSC (16 mg, 0.062 mmol), TEA (13 μL, 0.093 mmol), and DMAP (0.2 mg, 0.002 mmol) were dissolved in 1 mL of DMF was stirred for three hours at room temperature while shielded from light.

A coarse product was purified by HPLC, and 6JF646, NHS (5.6 mg, 0.0094 mmol) was acquired as a blue solid (yield: 61%).

¹H NMR (400 MHz, Acetone-d₆): δ 8.35 (dd, 1H, J=1.2, 8.0 Hz), 8.18 (dd, 1H, J=0.8, 8.0 Hz), 7.92-7.91 (m, 1H), 6.82-6.80 (m, 4H), 6.36 (dd, 2H, J=2.8, 8.8 Hz), 3.90 (t, 8H, J=7.2 Hz), 2.95 (s, 4H), 2.35 (quint, 4H, J=7.2 Hz), 0.63 (s, 3H), 0.53 (s, 3H). ¹³C NMR (100 MHz, Acetone-d₆): δ 170.3, 169.4, 162.0, 156.5, 152.2, 137.0, 132.1 (including two peaks), 131.4, 131.3, 128.9, 128.0, 127.5, 126.6, 116.5, 113.6, 52.7, 26.4, 17.3, 0.1, −0.9. HRMS (ESI⁺) calcd. for [M+H]⁺, 594.20549 found, 594.20799 (Δ2.50 mmu).

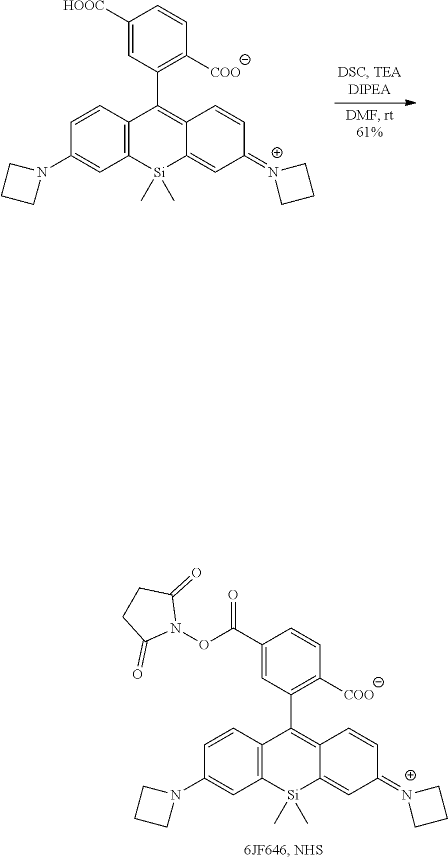

### Synthesis of 6JF646-DNP (2-(3-(azetidine-1-ium-1-ylidene)-7-(azetidine-1-yl)-5,5-dimethyl-3,5-dihydrodibenzo[b,e]silin-10-yl)-4-((2-(2-(2-((2,4-dinitrophenyl)amino)ethoxy)ethoxy)ethyl)carbamoyl)benzoate)

A reaction mixture in which 6JF646, NHS (5.0 mg, 0.0084 mmol), DNP-amine (5.3 mg, 0.017 mmol), and DIPEA (7.3 UL, 0.042 mmol) were dissolved in 0.5 mL of DMF was stirred for six hours at room temperature while shielded from light. A coarse product was purified by HPLC, and 6JF646-DNP (5.5 mg, 0.0069 mmol) was acquired as a green solid (yield: 82%).

¹H NMR (400 MHz, Acetone-d₆): δ 8.92 (d, 1H, J=2.8 Hz), 8.59 (br s, 1H), 8.24 (dd, 1H, J=2.8, 9.6 Hz), 8.10-8.07 (m, 1H), 7.96-7.94 (m, 2H), 7.77 (br s, 1H), 7.15 (d, 1H, J=9.6 Hz), 6.77 (d, 2H, J=2.4 Hz), 6.73 (d, 2H, J=8.8 Hz), 6.30 (dd, 2H, J=2.4, 8.8 Hz), 3.87 (t, 8H, J=7.2 Hz), 3.75 (t, 2H, J=5.2 Hz), 3.62-3.53 (m, 10H), 2.34 (quint, 4H, J=7.2 Hz), 0.64 (s, 3H), 0.51 (s, 3H). ¹³C NMR (100 MHz, Acetone-d₆): δ 169.9, 166.2, 155.8, 152.1, 149.6, 149.4, 141.2, 137.3, 136.6, 133.1, 131.1, 130.7, 129.3, 128.9, 126.3, 124.4, 124.1, 116.4, 116.0, 113.3, 71.1, 70.9, 70.1, 69.3, 52.8, 43.8, 43.7, 40.6, 40.5, 17.4, 0.3, −1.2. HRMS (ESI⁺) calcd. for [M+H]⁺, 793.30118; found, 793.30155 (Δ0.37 mmu).

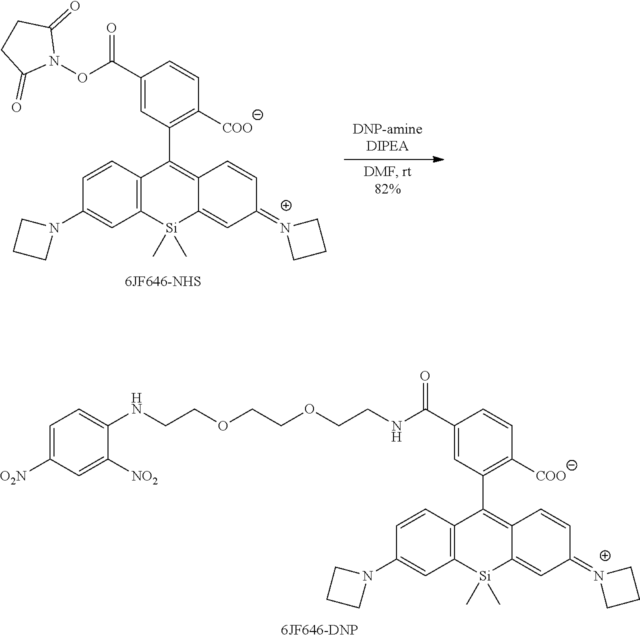

### Synthesis Example 14

### Synthesis of 6SiR600-DNP (2-(7-amino-3-imino-5,5-dimethyl-3,5-dihydrodibenzo[b,e]silin-10-yl)-4-((2-(2-(2-((2,4-dinitrophenyl)amino) ethoxy) ethoxy)ethyl)carbamoyl)benzoate)

A reaction mixture in which 6-carboxy-SiR600 (13 mg, 0.022 mmol), TSTU (7.9 mg, 0.026 mmol), and DIPEA (100 μL, 0.57 mmol) were dissolved in 1 mL of DMF was stirred for 15 minutes at room temperature while shielded from light. An acetonitrile solution (84 μL) of 1 M DNP-amine was dropped therein, and the mixture was further stirred for 30 minutes at room temperature while shielded from light. A coarse product was purified by HPLC, and an intermediate was acquired as a green solid. A reaction mixture in which the intermediate (14 mg, 0.016 mmol), tetrakis(triphenylphosphine)palladium(0) (2.4 mg, 0.0021 mmol), and 1,3-dimethylbarbituric acid (12 mg, 0.076 mmol) were dissolved in 5 mL of deoxygenated DCM was stirred all night at room temperature in an argon atmosphere. The reaction mixture was concentrated, and then purified by HPLC, and 6SiR600-DNP (3.7 mg, 0.0052 mmol) was acquired as a green solid (yield of the two reactions: 24%).

¹H NMR (400 MHz, Acetone-d₆+TFA): δ 8.89 (d, 1H, J=2.8 Hz), 8.25 (dd, 1H, J=9.6, 2.8 Hz), 8.16 (dd, 1H, J=8.0, 1.2 Hz), 8.10 (d, 2H, J=2.4 Hz), 8.04 (d, 1H, J=8.0 Hz), 8.00 (br s, 1H), 7.58 (dd, 2H, J=8.8, 2.4 Hz), 7.44 (d, 2H, J=8.8 Hz), 7.25 (d, 1H, J=9.6 Hz), 3.80 (t, 2H, J=5.6 Hz), 3.69-6.60 (m, 8H), 3.51 (t, 2H, J=5.6 Hz), 0.82 (s, 3H), 0.70 (s, 3H). HRMS (ESI⁺) calcd. for [M+H]⁺, 713.23858; found, 713.24096 (Δ2.38mmu).

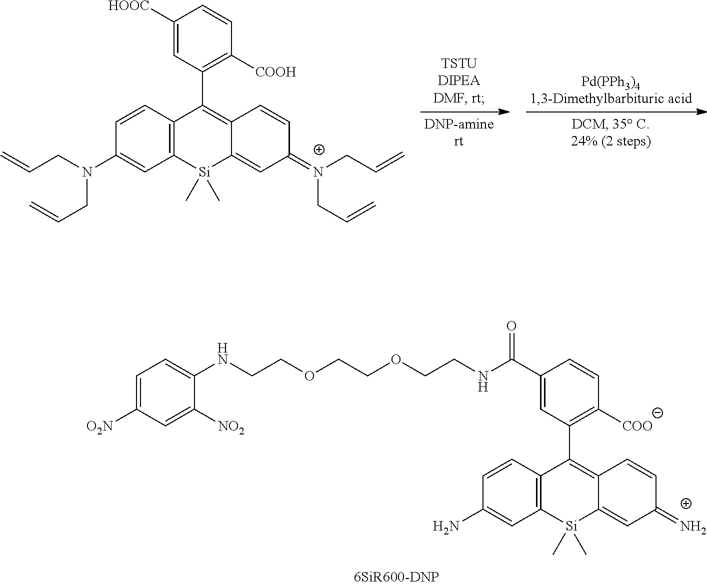

### Synthesis Example 15

### Synthesis of 6SiR700-DNP (4-((2-(2-(2-((2,4-dinitrophenyl)amino)ethoxy)ethoxy)ethyl)carbamoyl)-2-(1,9,11,11-tetramethyl-2,3,7,8,9,11-hexahydrosilino[3,2-f:5,6-f′]diindol-1-ium-5-yl)benzoate)

A reaction mixture in which 6-carboxy-SiR700 (Lukinavicius, G. et al. Fluorogenic probes for multicolor imaging in living cells. J. Am. Chem. Soc. 138, 9365-9368 (2016).) (8.3 mg, 0.016 mmol), TSTU (5.6 mg, 0.019 mmol), and DIPEA (71 μL, 0.40 mmol) were dissolved in 1 mL of DMF was stirred for 15 minutes at room temperature while shielded from light. An acetonitrile solution (59 μL) of 1 M DNP-amine was dropped therein, and the mixture was further stirred for 30 minutes at room temperature while shielded from light. A coarse product was purified by HPLC, and 6SiR700-DNP (8.6 mg, 0.011 mmol) was acquired as a green solid (yield: 70%).

¹H NMR (400 MHz, Acetone-d₆): δ 8.91 (d, 1H, J=2.4 Hz), 8.83 (br s, 1H), 8.28-8.24 (m, 1H), 8.05 (s, 2H), 7.95 (t, 1H, J=5.2 Hz), 7.68 (s, 1H), 7.18 (d, 1H, J=9.6 Hz), 6.97 (br s, 2H), 6.66 (s, 2H), 3.77 (t, 2H, J=5.2 Hz), 3.64-3.60 (m, 8H), 3.54-3.45 (m, 6H), 2.95 (s, 6H), 2.85-2.77 (m, 4H), 0.65 (s, 3H), 0.51 (s, 3H). ¹³C NMR (100 MHz, Acetone-d₆): δ 166.0, 165.9, 159.5, 156.0, 149.5, 149.4, 136.5, 133.9, 131.0, 130.8, 128.3, 124.4, 118.0, 116.2, 116.0, 115.2, 113.5, 71.1, 70.9, 70.1, 69.3, 55.5, 43.8, 43.7, 40.6, 40.5, 34.6, 27.9, −0.4, −1.2. HRMS (ESI⁺) calcd. for [M+H]⁺, 793.30118; found, 793.29938 (Δ-1.80 mmu).

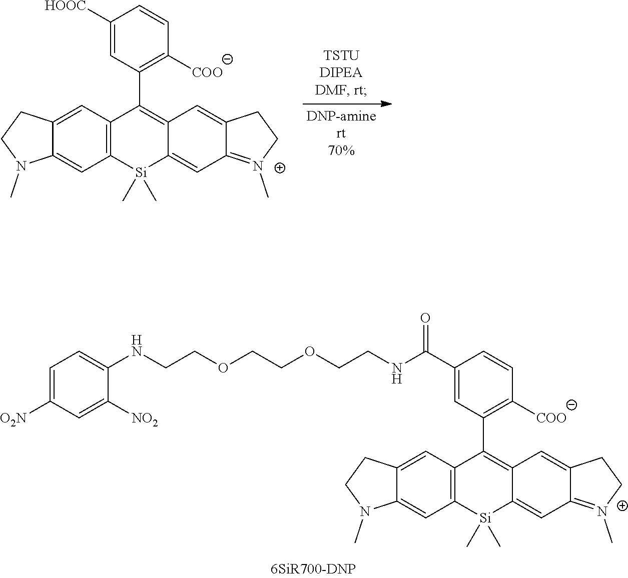

3. Measurement of Fluorescence Enhancement of Fluorophore-DNP by Supernatant of Anti-DNP-Antibody-Producing Hybridoma Culture

A 1 mM SRB-DNP/DMSO solution was diluted with PBS (pH 7.4) to prepare a 5 μM SRB-DNP/PBS solution, and 10 μL thereof was dispensed into each well of a 96-well plate (BD 353219 Imaging Plate). The SRB-DNP was identical to SR-DN1 reported in Sunbul et al., and was also synthesized by the same method (M. Sunbul, A. Jaschke, Contact-mediated quenching for RNA imaging in bacteria with a fluorophore-binding aptamer. Angewandte Chemie (International ed. in English) 52, 13401-13404 (2013).). The hybridoma clone culture solution or the hybridoma culture medium as a negative control (90 μL) was added and the mixture was stirred for 30 seconds at 1000 rpm using a Mixmate (Eppendorf), after which fluorescence was measured using an SH-9000 microplate reader (CORONA ELECTRIC CO., LTD.). Measurement wavelength condition: Ex/Em=570 nm/600 nm. For the hybridoma culture supernatant in the 27 wells exhibiting the greatest fluorescence change rate in this screening, the fluorescence enhancement effect was further investigated for four types of fluorophore-DNP pairs (SRB-DNP, 50G-DNP, R110-DNP, and DCF-DNP).

Cloning by limiting dilution was performed for the hybridomas of four wells (1E10, 1H4, 3B12, and 4C12) in which a significant fluorescence increase effect with respect to a plurality of types of fluorophore-DNP pairs was observed.

4. Cell Culture and Plasmid Introduction

HEK293T cells and HeLa cells were cultured in Dulbecco's Modified Eagle's Medium (DMEM, Wako) including 10% fetal bovine serum (FBS, SIGMA) at a carbon dioxide concentration of 5% and a temperature of 37° C.

Gene transfer into HeLa cells was performed using lipofection. For the HeLa cells under culture in a 24-well dish, 25 μL of Opti-MEM I (GIBCO) to which 0.5 μg of a plasmid had been added was added to a mixed solution of 2 μL of Lipofectamine 2000 (Invitrogen) and 25 μL of Opti-MEM I and incubated for five minutes at room temperature. This mixture was added to 500 μL of a medium 90-100% confluent with HEK293T cells and HeLa cells, and culturing was performed at 37° C. at a carbon dioxide concentration of 5%. Cell concentration was diluted to 1/10 5 to 8 hours after transfection, and cells were re-seeded on a glass dish. Cells were subjected to various imaging experiments after 24 hours had passed since transfection.

5. Live Cell Imaging

Cells were observed using an inverted microscope (IX-71, Olympus) provided with a xenon arc lamp. Images were captured using an EM-CCD camera (iXon EM+, Andor). During acquisition of a 6SiR-DNP fluorescence image, a Cy5-4040C filter set (Semrock) comprising a 608-648 nm excitation light filter, a 660 nm dichroic mirror, and a 672-712 nm absorption filter was used.

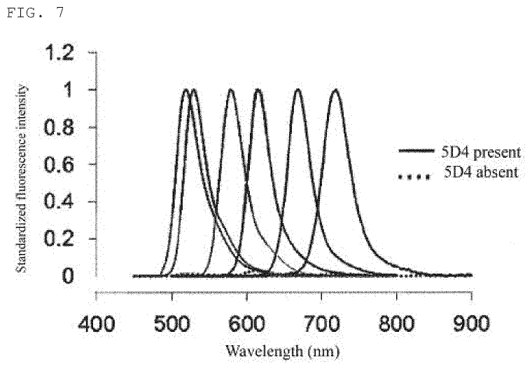

During acquisition of a CFP fluorescence image, a U-MCFPHQ filter set (Olympus) comprising a 424-438 nm excitation light filter, a 450 nm dichroic mirror, and a 460-510 nm absorption filter was used. An objective lens (10×NA 0.3, 20×NA 0.75: Olympus) was used for the screening in FIG. 3, and an oil immersion objective lens (100× NA 1.4: Olympus) was used in the observations in FIGS. 5, 8, 9, and 10.

The HeLa cell medium was drawn up and washed with HBS buffer solution (25 mM HEPES, 125 mM NaCl, 2.5 mM KCl, 2 mM CaCl₂, 1 mM MgCl₂, and 25 mM D-glucose; pH 7.4), and cells were then observed in the HBS buffer solution including 6SiR-DNP at a concentration of 100 nM or 10 nM. Image capture was started 5 minutes after the 6SiR-DNP was added. Images were analyzed using ImageJ (NIH).

6. SIM Imaging

HeLa cells were transfected with pTBB-GGGSR-5D4, and after being stripped by trypsin treatment 5-8 hours later, the cells were re-seeded at a density of 1/10 on a cover glass coated with collagen and poly-L-lysine and further cultured for 18-25 hours at 37° C. in the presence of 4% CO₂, and subjected to SIM imaging. A structured illumination image was acquired using a SIM system (Nikon). A 640-nm semiconductor laser was used for excitation, and a fluorescence image was acquired at a two-second frame rate using an objective lens (100× SR Apo TIRF, NA 1.49: Nikon) and an s-CMOS camera (ORCA Flash 4, Hamamatsu). The acquired fluorescence image was analyzed using NIS-Elements software (Nikon).

7. Experimental Results

### Example 1

Acquisition of Anti-DNP scFv

Anti-DNP monoclonal antibodies were prepared using mice (see Experimental Methods). In screening of antibody-producing hybridomas, antibody titers in the hybridoma culture supernatant were evaluated by ELISA (FIG. 3A). As a result, it was confirmed that a large amount of anti-DNP antibodies were produced. The rate of increase of fluorescence in the fluorophore-DNP pair (SRB-DNP) by the hybridoma culture supernatant was also evaluated to acquire an svFv having good efficiency of removal of fluorescence quenching (FIG. 3B).

The fluorescence enhancement effect was investigated for four types of fluorophore-DNP pairs in the hybridoma culture supernatant in the 27 wells exhibiting the greatest fluorescence change rate in the screening so far, and the hybridomas were cloned from four wells (1E10, 1H4, 3B12, and 4C12) in which a significant fluorescence increase effect was observed with respect to a plurality of types of fluorophore-DNP pairs (FIG. 3C). The four types of fluorophore-DNP pairs used herein were SRB-DNP, 50G-DNP, R110-DNP, and DCF-DNP.

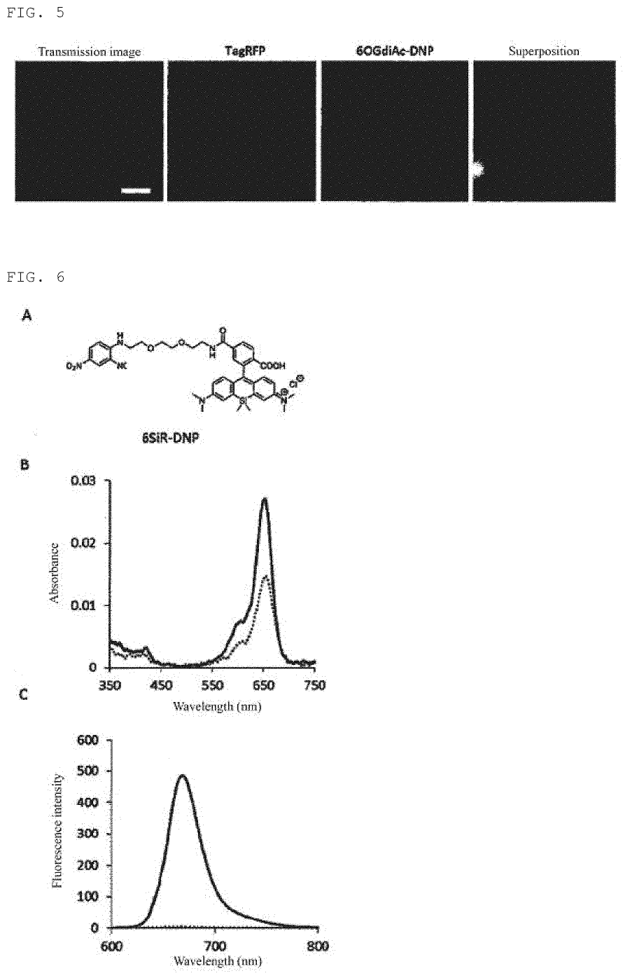

RNA was extracted from the hybridomas of the resultant four clones, and cDNA fragments of the variable regions of the light chains and heavy chains of monoclonal antibodies were obtained by reverse transcription. The cDNA fragments of the variable regions of the light chains and heavy chains were connected via a linker sequence by overlap PCR, and scFv constructs were constructed only from subclones (4E10 and 5D4) derived from wells 1E10 and 3B12, respectively. Recombinant proteins of 4E10 and 5D4 were expressed/purified in an E. coli expression system, and when the effect of the fluorophore-DNP pair (DCF-DNP) on change in fluorescence intensity was investigated, only 5D4 was found to exhibit an increase (of about 10 times) in fluorescence intensity. The nucleic acid sequence of the cDNA sequence coding for 5D4 was identified by sequencing (SEQ ID NO: 8), an amino acid sequence determined from the result thereof was analyzed using the IMGT database (http://www.imgt.org/), and a DCR region was specified (FIG. 4).

### Example 2

Expression Test of Anti-DNP scFv in Cultured Cells

The 5D4 clone for which a fluorescence-increasing effect of the scFv on the fluorophore-DNP pair was observed was expressed in HEK293T cells as a fusion protein with the fluorescent protein TagRFP, and the state of expression of the scFv in the cells or the propriety of fluorescent labeling of cytoplasm by the fluorophore-DNP pair was evaluated. Cells were loaded with 6OGdiac-DNP as the fluorophore-DNP pair to give a final concentration of 1 μM and left for 10 minutes at room temperature, and the 6OGdiAc-DNP outside the cells was then washed with HBS. The cells were then left for 10 minutes at 37° C. and subsequently observed using a fluorescence microscope, in which green fluorescence was confirmed only in the cytoplasm of TagRFP-positive HEK293T cells (FIG. 5). It was confirmed that the obtained 5D4 clone can be expressed in a cell in a state of functioning as an anti-DNP scFv, and the 5D4 clone was therefore subjected to a further development process.

### Example 3

Analysis of Fluorescence Change Characteristics of Fluorophore-DNP Pair

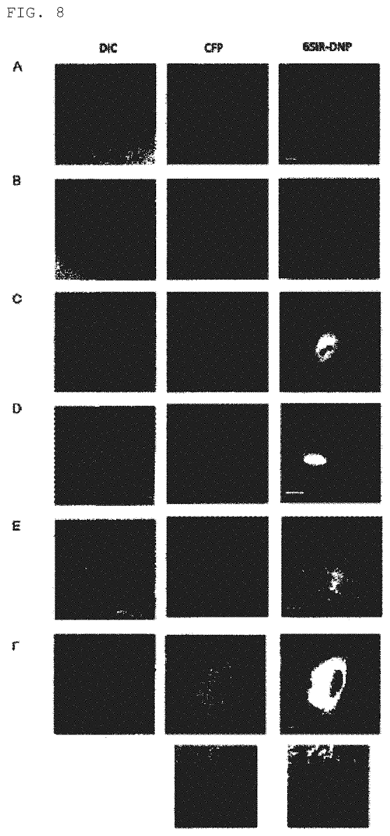

From among the fluorophore-DNP pairs prepared as described above, an absorption spectrum and a fluorescence spectrum of 6SiR-DNP, which exhibits fluorescence in a near-infrared region and in which a significant reduction of an effect of autofluorescence in application to a cell can be anticipated, were measured in the presence and absence of 5D4. When 5D4 was present, 6SiR-DNP exhibited an absorption maximum at 653 nm and a fluorescence maximum at 668 nm (FIGS. 6A and 6B). This fluorescence characteristic is similar to that of Cy5 dye, which is widely used in fluorescence imaging in cells. In the presence of 5D4, the fluorescence intensity of 6SiR-DNP was increased by a factor of 98 relative to the fluorescence intensity thereof in the absence of 5D4 (FIG. 6B). When fluorescence quantum yield of 6SiR-DNP in a state in which 6SiR-DNP is bound to 5D4 was measured, the fluorescence quantum yield in the presence of an excess of 5D4 was 0.57. This value for the fluorescence quantum yield is large, and is equal to or greater than that of Cy5 (quantum yield=0.2-0.4) and Cy5.5 (quantum yield=0.24), which are also highly versatile fluorescent dyes in cell labeling experiments (Q. Zheng et al., Ultra-stable organic fluorophores for single-molecule research. Chem Soc Rev 43, 1044-1056 (2014).). The other fluorophore-DNP pairs were also found to exhibit a similarly large increase in fluorescence intensity in the presence of 5D4 (FIG. 7). FIG. 7 shows the fluorescence spectra in the presence and absence of 5D4 of 60G-DNP, 6DCF-DNP, 6JF549-DNP, 6SiR600-DNP, 6SiR-DNP, and 6SiR700-DNP, in this order from the short wavelength side of FIG. 7.

### Example 4

Fluorescent Labeling of 5D4 Expressed at an Arbitrary Site in a Cultured Cell

5D4 expressed at an arbitrary site in a cell was labeled with 6SiR-DNP, and the ability to observe a fluorescence image using a fluorescence microscope was investigated. When cells expressing only ECFP were loaded with 6SiR-DNP (FIG. 8A), and when ECFP to which 5D4 was added was expressed in cells but the cells were not loaded with 6SiR-DNP, a fluorescence signal due to 6SiR-DNP was not observed (FIG. 8B). When 5D4 was expressed in Hela cells as a fusion protein with ECFP, a 6SiR-DNP fluorescence signal was observed covering the entire cytoplasm (FIG. 8C), the same as when 5D4 was expressed as a fusion protein with TagRFP (FIG. 5). 5D4 was expressed as a fusion protein with ECFP and a localization peptide for each of the nucleus, the cell membrane, and the endoplasmic reticulum, after which the cells were loaded with 6SiR-DNP at a final concentration of 0.1 μM, and fluorescence images were acquired in which fluorescent labeling at the targeted intracellular sites was accomplished for all the cells (FIGS. 8D, 8E, and 8F).

The above results indicate that 5D4 is stably expressed at arbitrary sites in a cell and does not lose ability to bind with DNP, and that the fluorophore-DNP pair is fluorescent only when 5D4 and DNP are bound. From the above, it was found that fluorescence imaging of intracellular organelle structure at high contrast is possible without washing of the fluorophore-DNP pair and even in the presence of unreacted 6SiR-DNP in an extracellular fluid.

### Example 5

Labeling of 5D4 Expressed as a Fusion Protein with an Arbitrary Protein

It was investigated whether a protein expressed as a fusion protein with 5D4 in a cell can be labeled with a fluorophore-dye pair.

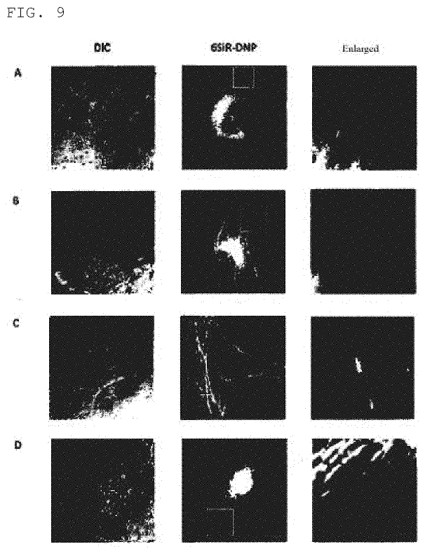

Using a β-tubulin protein as an observation object, a 5D4 fusion protein expression construct was transgenically introduced into a Hela cell. When the cell was observed using a fluorescence microscope under a condition in which 6SiR-DNP was present in the extracellular fluid, a fibrous structure characteristic of tubulin was observed (FIG. 9A). An attempt was also made to observe b-actin in the cell. When a fusion protein of β-actin and 5D4 was expressed in a HeLa cell and the cell was observed, a fibrous structure characteristic of β-actin was observed (FIG. 9B). In order to visualize F-actin internal to the cell, an expression construct in which a peptide sequence (Lifeact sequence) for specifically binding to F-actin was added to the N-terminus of 5D4 was transgenically introduced into a HeLa cell. A characteristic fibrous structure was observed that was similar to the structure observed when a fusion protein of β-actin and 5D4 was expressed in the cell (FIG. 9C).

A fusion protein of 5D4 and a STIM1 protein (Y. Baba et al., Coupling of STIM1 to store-operated Ca2+ entry through its constitutive and inducible movement in the endoplasmic reticulum. Proceedings of the National Academy of Sciences of the United States of America 103, 16704-16709 (2006)) localized on the endoplasmic reticulum and known to control calcium signaling was expressed in a HeLa cell, and when the fusion protein was stained with 6SiR-DNP, a fluorescence image of a structure running along the endoplasmic reticulum and microtubules was observed (FIG. 9D). This result agrees with the intracellular distribution of STIM1 reported in prior research.

The results of the protein labeling experiment described above indicate that 5D4 can be used as a molecular tag capable of fluorescent labeling and expression in a cell as a fusion protein with a target molecule for observation in the cell.

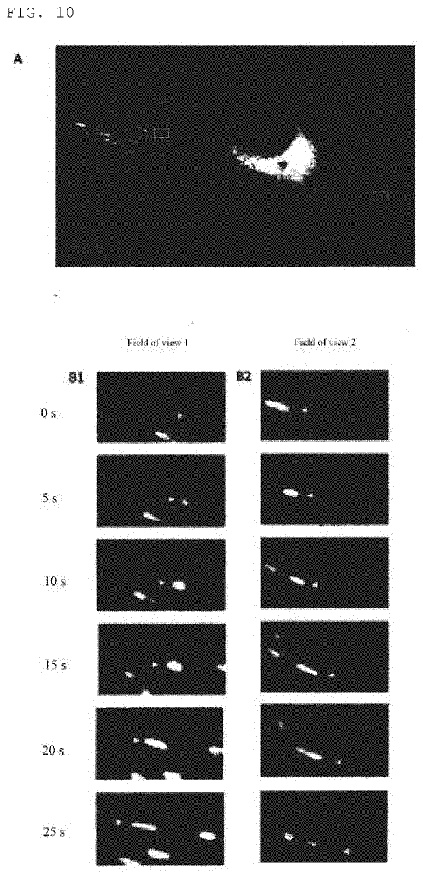

Applicability to time-lapse imaging of a protein fluorescently labeled using 5D4 was verified through kinetic observation of a STIM1 protein. Movement of a STIM1 protein to which 5D4 was added along a microtubule in the same manner as the reported GFP-STIM1 was observed, the STIM1 to which 5D4 was added having been fluorescently labeled with 6SiR-DNP (FIG. 10). The above results indicate that by using the above molecular tag, live cell imaging of a target protein can be performed, and visualization analysis of intracellular protein dynamics is possible.

### Example 6

Live Cell Super-Resolution Imaging

Development of super-resolution microscope techniques in recent years is advancing efforts to analyze the spatiotemporal dynamics of functional molecules or intracellular organelle microstructures at nanometer resolution and with high precision. Applicability of the present invention to live cell super-resolution imaging by structured illumination microscopy (SIM), which is one super-resolution imaging technique, was verified. When a fusion protein of 5D4 and tubulin was expressed in a HeLa cell, and the results of observation by a normal fluorescence microscope and SIM were compared, structures that could not be spatially separated in a normal fluorescence image were observable as being constituted from a plurality of fibrous structures (FIGS. 11A and 11B). When time-lapse imaging by SIM was performed, the structure of tubulin was stably observable for about 150 seconds (FIG. 11C). It was also possible to detect a change in a tubulin microstructure at a temporal resolution of 30 seconds (FIG. 11D). The above results indicated that labeling of intracellular molecules using the molecular tag technique developed in the present research is successful in live cell imaging of the nanoscale microstructure of tubulin by SIM, is applicable to real-time imaging, and can be utilized for continuous and high-precision dynamic analysis of intracellular molecules.

In the present invention, by causing only a tagged molecule as an analysis object to emit fluorescence in a cell, fluorescence observation of an intracellular molecule or organelle is made possible with extremely low background fluorescence. In the case of Halo tagging or SNAP tagging, a dye in which fluorescence is always ON is used, and an operation for washing away the dye is therefore necessary in order to observe with low background fluorescence. In these techniques, nonspecific adsorption inside and outside the cell is also immediately reflected in a fluorescence observation image as a fluorescence signal, and a means of reducing background fluorescence is also necessary. An advantage that a DNP tagging technique has over the existing molecular tagging techniques is that fluorescence observation with low background fluorescence can conveniently be performed merely by adding a fluorophore-dye pair to the extracellular fluid. An important feature of the DNP tagging technique is also the applicability thereof to time-lapse imaging or super-resolution imaging in live cell imaging. Fluorescence imaging by 6SiR-DNP, which emits near-infrared fluorescence, exhibits high tissue permeability and low autofluorescence in comparison with GFP fluorescence, and is therefore highly useful for fluorescence imaging of tissues as well.

In fluorescence imaging, particularly in fluorescence imaging experiments by laser microscope and other fluorescence imaging that requires irradiation of a cell location with strong excitation light, bleaching of a fluorescent dye can pose a significant obstacle to high-precision imaging or imaging that is performed over a long period of time. Application of intense excitation light or image capture under prolonged application of excitation light is necessary to obtain a high signal-to-noise ratio for observation, but the dye is bleached when intense excitation light is applied for a long time, and fluorescence imaging cannot be performed for a long time with a sustained high signal-to-noise ratio. In the fluorescence imaging using 5D4 according to the examples of the present invention, because the labeling method does not involve covalent bonding, in contrast with Halo tagging or SNAP tagging, the fluorophore-dye pair is thought to dissociate after being bleached and losing function. A process whereby unreacted fluorophore-dye in the surrounding area after dissociation of the fluorophore-dye pair re-binds with 5D4 and attains a fluorescence-ON state can be expected to repeat, and the present invention is therefore considered to be suitable for long time-lapse imaging as well. It is suggested that continuous image acquisition using excitation light having high laser intensity is actually possible in SIM imaging.

The compound 6SiR-DNP, which is one of the compounds of the present invention, is thoroughly quenched when not bound to 5D4. Consequently, the effect of fluorescence originating from 6SiR-DNP that is not bound to 5D4 even when present outside the cell on spatial resolution in observation of a target molecule or organelle for observation is suppressed to a negligible level. The fact that there is no need for a step for removing an unnecessary fluorescent dye from the system during fluorescence observation is particularly useful in high-throughput screening (HTS) for drug discovery and the like. In HTS, efficiency of the screening system as a whole is increased by reducing the number of steps such as probe washing, and numerous specimens are required to be assayed at extremely high efficiency. A method in which washing and other processing is omitted and reaction and measurement are performed successively is referred to as a “mix and measure” or “homogeneous” method, and such a method is considered desirable particularly in drug screening in which tens of thousands to hundreds of thousands of compounds are assayed. From the knowledge obtained through the present invention, in a screening system in which a DNP tag and a fluorophore-dye pair are introduced, an HTS system can be constructed in which there is no need for a washing process for excess fluorescent dye.

### Example 6

Super-Resolution Imaging by Single-Molecule Localization

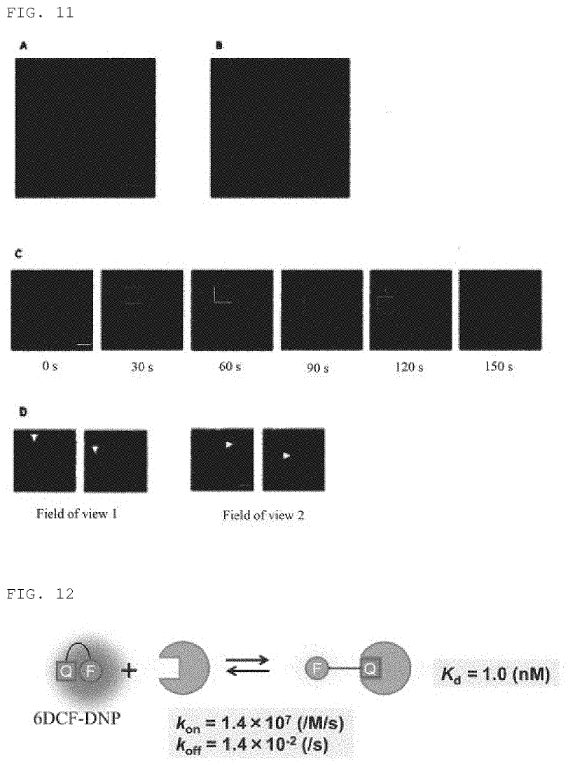

In applying the molecular tag (De-QODE tag) of the present invention to super-resolution imaging by molecular localization, the realization of fluorescence intermittency by control of De-QODE tag-probe binding/dissociation kinetics was investigated. FIG. 12 is a schematic diagram illustrating the binding/dissociation kinetics of 6DCF-DNP and 5D4, and in this case, a dissociation constant (k_{off}) for a fluorescence-OFF state is 1.4×10⁻² (/s). Molecular modification of both the De-QODE tag and the probe to increase the De-QODE tag-probe dissociation constant (k_{off}) was investigated.

(1) Anti-DNP scFv Mutant Protein Expression Construct

An amino acid mutation was introduced into an MBP-scFv protein coding region by circular PCR with pMalc5E-5D4 as a template, using a thermostable polymerase KOD Plus (TOYOBO) and forward and reverse primers including a codon modified to correspond to the desired amino acid mutation. The resultant linear PCR product was circularized using a Ligation Kit Version 2 (TAKARA), and an MBP-scFv mutant expression construct was obtained.

### (2) Probe Synthesis

### Synthesis of pCNoNP-Amine (4-((2-(2-(2-aminoethoxy)ethoxy)ethyl)amino)-3-nitrobenzonitrile)

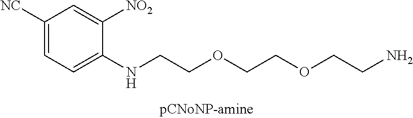 [SMILES: N#Cc1ccc(NCCOCCOCCN)c([N+](=O)[O-])c1]

By the same scheme used in the synthesis of DNP-amine, pCNoNP-amine (1.22 g, 4.14 mmol) was acquired as a yellow oil (yield: 75%) using 4-bromo-3-nitrobenzonitrile as a starting material. ¹H NMR (400 MHz, CD₃OD): δ 9.89 (br s, 1H), 9.83 (d, 1H, J=2.0 Hz), 9.02 (dd, 1H, J=9.2, 2.0 Hz), 8.44 (d, 1H, J=9.2 Hz), 5.10 (t, 2H, J=5.2 Hz), 5.00-4.88 (m, 6H), 4.76 (t, 2H, J=5.2 Hz), 4.06 (t, 2H, J=5.2 Hz). ¹³C NMR (100 MHz, CD₃OD): δ 196.1, 186.2, 180.6, 166.7, 166.0, 164.3, 145.9, 122.0, 118.8, 118.6, 117.0, 91.3, 90.2. HRMS (ESI) calcd for [M+Na]⁺, 317.12203; found, 317.12552 (Δ3.49 mmu).

### Synthesis of pBroNP-Amine (N-(2-(2-(2-aminoethoxy)ethoxy)ethyl)-4-bromo-2-nitroaniline)

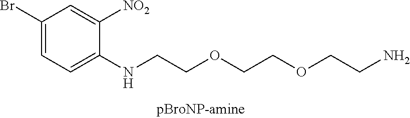 [SMILES: NCCOCCOCCNc1ccc(Br)cc1[N+](=O)[O-]]

By the same scheme used in the synthesis of DNP-amine, pBroNP-amine (1.36 g, 3.89 mmol) was acquired as an orange-colored oil (yield: 77%) using 4-bromo-1-fluoro-2-nitrobenzene as a starting material.

¹H NMR (400 MHz, CD₃OD): δ 8.21 (d, 1H, J=2.4 Hz), 7.55 (dd, 1H, J=9.2, 2.4 Hz), 7.99 (d, 1H, J=9.6 Hz), 3.77 (t, 2H, J=5.6 Hz), 3.70-3.68 (m, 2H), 3.66-3.63 (m, 2H), 3.52 (t, 2H, J=5.6 Hz), 2.77 (t, 2H, J=5.26 Hz). ¹³C NMR (100 MHz, CD₃OD): δ 145.7, 139.9, 133.3, 129.4, 117.5, 107.0, 73.6, 71.5, 71.4, 70.0, 43.6, 42.1. HRMS (ESI⁺) calcd. for [M+H]⁺, 348.05535; found, 348.05503 (Δ0.32 mmu).

### Synthesis of pCloNP-Amine (N-(2-(2-(2-aminoethoxy)ethoxy)ethyl)-4-chloro-2-nitroaniline)

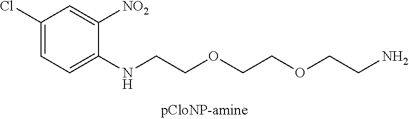 [SMILES: NCCOCCOCCNc1ccc(Cl)cc1[N+](=O)[O-]]

By the same scheme used in the synthesis of DNP-amine, pCloNP-amine (1.17 g, 3.85 mmol) was acquired as an orange-colored oil (yield: 69%) using 4-chloro-1-fluoro-2-nitrobenzene as a starting material.

¹H NMR (400 MHz, CD₃OD): δ 8.03 (d, 1H, J=9.2 Hz), 6.99 (d, 1H, J=2.0 Hz), 6.59 (dd, 1H, J=9.2, 2.0 Hz), 3.77 (t, 2H, J=5.2 Hz), 3.70-3.64 (m, 4H), 3.52 (t, 2H, J=5.2 Hz), 3.47 (t, 2H, J=5.2 Hz), 2.78 (t, 2H, J=5.2 Hz). ¹³C NMR (100 MHz, CD₃OD): δ 147.1, 143.6, 131.7, 129.1, 116.6, 114.7, 73.5, 71.5, 71.3, 70.0, 43.7, 42.1. HRMS (ESI⁺) calcd. for [M+H]⁺, 304.10586; found, 304.10603 (Δ0.17 mmu).

### Synthesis of pMeOoNP-Amine

(N-(2-(2-(2-aminoethoxy)ethoxy)ethyl)-4-methoxy-2-nitroaniline

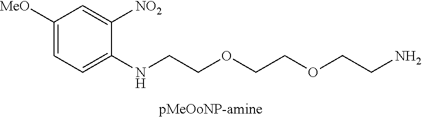 [SMILES: COc1ccc(NCCOCCOCCN)c([N+](=O)[O-])c1]

By the same scheme used in the synthesis of DNP-amine, pMeOoNP-amine (1.01 g, 3.39 mmol) was acquired as a red oil (yield: 69%) using 1-fluoro-4-methoxy-2-nitrobenzene as a starting material.

¹H NMR (400 MHz, CD₃OD): δ 7.45 (d, 1H, J=2.8 Hz), 7.12 (dd, 1H, J=9.2, 2.8 Hz), 6.89 (d, 1H, J=9.2 Hz), 3.75-3.73 (m, 5H), 3.67-3.61 (m, 4H), 3.50 (t, 2H, J=5.2 Hz), 3.44 (t, 2H, J=5.2 Hz), 2.77 (t, 2H, J=5.2 Hz). ¹³C NMR (100 MHz, CD₃OD): δ 150.9, 142.4, 131.9, 128.0, 116.7, 107.7, 73.6, 71.5, 71.3, 70.2, 56.2, 43.7, 42.2. HRMS (ESI⁺) calcd. for [M+H]⁺, 300.15540; found, 300.15689 (Δ1.49 mmu).

### Synthesis of pCF3oNP-Amine (N-(2-(2-(2-aminoethoxy) ethoxy)ethyl)-2-nitro-4-(trifluorometh yl) aniline)

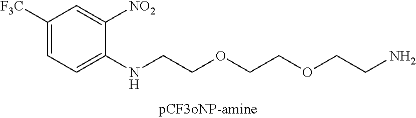 [SMILES: NCCOCCOCCNc1ccc(C(F)(F)F)cc1[N+](=O)[O-]]

By the same scheme used in the synthesis of DNP-amine, pCF3oNP-amine (1.50 g, 4.45 mmol) was acquired as a yellow oil (yield: 77%) using 1-fluoro-2-nitro-4-(trifluoromethyl)benzene as a starting material.

¹H NMR (400 MHz, CD₃OD): δ 8.36 (d, 1H, J=1.2 Hz), 7.67 (dd, 1H, J=9.2, 2.4 Hz), 7.18 (d, 1H, J=9.2 Hz), 3.79 (t, 2H, J=5.2 Hz), 3.71-3.64 (m, 4H), 3.59 (t, 2H, J=5.2 Hz), 3.52 (t, 2H, J=5.2 Hz), 2.77 (t, 2H, J=5.2 Hz). ¹³C NMR (100 MHz, CD₃OD): δ 148.3, 132.9 (q, J=3.2 Hz), 132.0, 125.8 (q, J=4.3 Hz), 125.3 (q, J=268.3 Hz), 117.8 (q, J=33.7 Hz), 116.6, 73.6, 71.6, 71.4, 69.9, 43.7, 42.1. HRMS (ESI⁺) calcd. for [M+H]⁺, 338.13222; found, 338.13308 (Δ0.86 mmu).

### Synthesis of pCOOMeoNP-Amine Methyl (4-((2-(2-(2-aminoethoxy)ethoxy)ethyl)amino)-3-nitrobenzoate

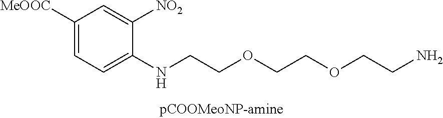 [SMILES: COC(=O)c1ccc(NCCOCCOCCN)c([N+](=O)[O-])c1]

By the same scheme used in the synthesis of DNP-amine, pCOOMeoNP-amine (1.60 g, 4.90 mmol) was acquired as a yellow oil (yield: 82%) using methyl 4-fluoro-3-nitrobenzoate as a starting material.

¹H NMR (400 MHz, CD₃OD): δ 8.63 (d, 1H, J=2.0 Hz), 7.93 (dd, 1H, J=9.2, 2.0 Hz), 7.01 (d, 1H, J=9.2 Hz), 3.86 (s, 1H), 3.79 (t, 2H, J=5.2 Hz), 3.71-3.69 (m, 2H), 3.66-3.64 (m, 2H), 3.56 (t, 2H, J=5.2 Hz), 3.52 (t, 2H, J=5.2 Hz), 2.77 (t, 2H, J=5.2 Hz). ¹³C NMR (100 MHz, CD₃OD): δ 167.0, 148.9, 136.9, 132.3, 129.9, 117.8, 115.3, 73.6, 71.5, 71.4, 69.8, 52.6, 43.7, 42.2. HRMS (ESI⁺) calcd. for [M+H]⁺, 328.15031; found, 328.15102 (Δ0.71 mmu).

### Synthesis of pCF30oNP-Amine (N-(2-(2-(2-aminoethoxy) ethoxy)ethyl)-2-nitro-4-(trifluoromethoxy)aniline)

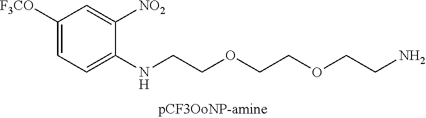 [SMILES: NCCOCCOCCNc1ccc(OC(F)(F)F)cc1[N+](=O)[O-]]

By the same scheme used in the synthesis of DNP-amine, pCF30oNP-amine (1.17 g, 3.31 mmol) was acquired as an orange-colored oil (yield: 73%) using 1-fluoro-2-nitro-4-(trifluoromethoxy)benzene as a starting material.

¹H NMR (400 MHz, CD₃OD): δ 7.99 (dd, 1H, J=2.8, 0.8 Hz), 7.43 (ddd, 1H, J=9.2, 2.8, 0.8 Hz), 7.11 (d, 1H, J=9.2 Hz), 3.79 (t, 2H, J=5.2 Hz), 3.71-3.64 (m, 4H), 3.56-3.52 (m, 4H), 2.79 (t, 2H, J=5.2 Hz). ¹³C NMR (100 MHz, CD₃OD): δ 145.7, 135.2 (q, J_{C-F}=571.9 Hz), 131.1, 122.0 (J_{C-F}=254.0 Hz), 120.0, 117.0, 73.6, 71.5, 71.3, 70.0, 43.8, 42.2. HRMS (ESI⁺) calcd. for [M+H]⁺, 354.12713; found, 354.12835 (Δ1.22 mmu).

### Synthesis of pSo2MeoNP-Amine (N-(2-(2-(2-aminoethoxy)ethoxy)ethyl)-4-methylsulfonyl)-2-nitroaniline)

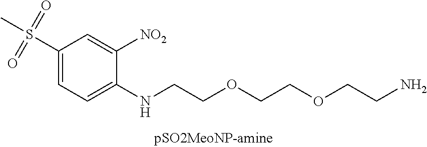 [SMILES: CS(=O)(=O)c1ccc(NCCOCCOCCN)c([N+](=O)[O-])c1]

By the same scheme used in the synthesis of DNP-amine, pSO2MeoNP-amine (0.855 g, 2.46 mmol) was acquired as an orange-colored oil (yield: 53%) using 1-fluoro-4-(methylsulfonyl)-2-nitrobenzene as a starting material.

¹H NMR (400 MHz, CD₃OD): δ 8.57 (d, 1H, J=2.4 Hz), 7.87 (dd, 1H, J=9.2, 2.4 Hz), 7.19 (d, 1H, J=9.2 Hz), 3.80 (t, 2H, J=5.2 Hz), 3.71-3.63 (m, 4H), 3.61 (t, 2H, J=5.2 Hz), 3.52 (t, 2H, J=5.2 Hz), 3.13 (s, 3H), 2.77 (t, 2H, J=5.2 Hz). ¹³C NMR (100 MHz, CD₃OD): δ 149.0, 134.6, 131.9, 128.5, 127.7, 116.7, 73.5, 71.5, 71.3, 69.8, 44.6, 43.8, 42.1. HRMS (ESI⁺) calcd. for [M+H]⁺, 348.12238; found, 348.12210 (Δ-0.28 mmu).

### Synthesis of pMeoNP-Amine (N-(2-(2-(2-aminoethoxy)ethoxy)ethyl)-4-methyl-2-nitroaniline)

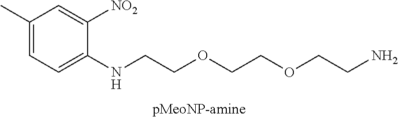 [SMILES: Cc1ccc(NCCOCCOCCN)c([N+](=O)[O-])c1]

By the same scheme used in the synthesis of DNP-amine, pMeoNP-amine (1.18 g, 4.17 mmol) was acquired as an orange-colored oil (yield: 64%) using 1-fluoro-4-methyl-2-nitrobenzene as a starting material.

¹H NMR (400 MHz, CD₃OD): δ 7.84 (d, 1H, J=0.8 Hz), 7.29-7.26 (m, 1H), 6.87 (d, 1H, J=8.8 Hz), 3.75 (t, 2H, J=5.2 Hz), 3.69-3.63 (m, 4H), 3.52 (t, 2H, J=5.2 Hz), 3.46 (t, 2H, J=5.2 Hz), 2.77 (t, 2H, J=5.2 Hz). ¹³C NMR (100 MHz, CD₃OD): δ 144.9, 138.8, 132.6, 126.6, 126.1, 115.3, 73.6, 71.5, 71.3, 70.1, 43.6, 42.2, 20.0. HRMS (ESI⁺) calcd. for [M+H]⁺, 284.16048; found, 284.16183 (D1.35 mmu).

### Synthesis of mCF3oNP-Amine (N-(2-(2-(2-aminoethoxy)ethoxy)ethyl)-2-nitro-5-(trifluoromethyl)aniline)

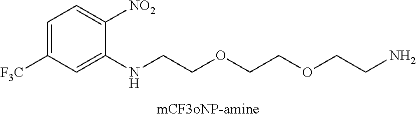 [SMILES: NCCOCCOCCNc1cc(C(F)(F)F)ccc1[N+](=O)[O-]]

By the same scheme used in the synthesis of DNP-amine, mCF3oNP-amine (1.12 g, 3.33 mmol) was acquired as an orange-colored oil (yield: 68%) using 2-fluoro-1-nitro-4-(trifluoromethyl)benzene as a starting material.

¹H NMR (400 MHz, CD₃OD): δ 8.22 (dd, 1H, J=8.8, 0.4 Hz), 7.28 (d, 1H, J=0.4 Hz), 6.85 (dd, 1H, J=8.8, 2.0 Hz), 3.80 (t, 2H, J=5.6 Hz), 3.72-3.64 (m, 4H), 3.56 (t, 2H, J=5.2 Hz), 3.52 (t, 2H, J=5.2 Hz), 2.78 (t, 2H, J=5.2 Hz). ¹³C NMR (100 MHz, CD₃OD): δ 146.3, 137.8 (q, J_{C-F}=32.2 Hz), 134.6, 128.8, 124.7 (q, J_{C-F}=271.1 Hz), 112.9 (q, J_{C-F}=4.1 Hz), 111.9 (q, J_{C-F}=3.3 Hz), 73.6, 71.6, 71.3, 70.2, 43.7, 42.2. HRMS (ESI⁺) calcd. for [M+H]⁺, 338.13222; found, 338.13335 (M1.13 mmu).

### Synthesis of mCNoNP-Amine (3-((2-(2-(2-aminoethoxy)ethoxy)ethyl)amino)-4-nitrobenzonitrile)

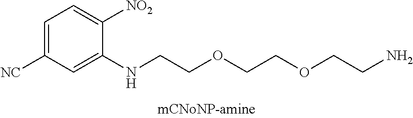 [SMILES: N#Cc1ccc([N+](=O)[O-])c(NCCOCCOCCN)c1]

By the same scheme used in the synthesis of DNP-amine, mCNoNP-amine (1.40 g, 4.74 mmol) was acquired as an orange-colored oil (yield: 97%) using 3-fluoro-4-nitrobenzonitrile as a starting material.

¹H NMR (400 MHz, (CD₃)₂CO): δ 8.29 (br s, 1H), 8.22 (d, 1H, J=8.4 Hz), 7.57 (d, 1H, J=1.6 Hz), 6.98 (dd, 1H, J=8.8, 1.6 Hz), 3.82 (t, 2H, J=5.2 Hz), 3.68-3.60 (m, 8H), 3.33-3.29 (m, 2H). ¹³C NMR (100 MHz, (CD₃)²CO): δ 145.9, 134.4, 128.4, 120.4, 119.6, 118.2, 117.6, 72.3, 71.2, 71.1, 69.7, 52.0, 43.6. HRMS (ESI⁺) calcd. for [M+Na]⁺, 317.12203; found, 317.12050 (Δ1.53 mmu).

### Synthesis of mMeOoNP-Amine (N-(2-(2-(2-aminoethoxy)ethoxy)ethyl)-5-methoxy-2-nitroaniline

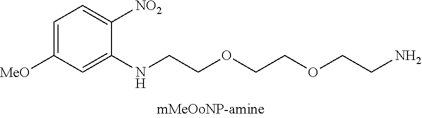 [SMILES: COc1ccc([N+](=O)[O-])c(NCCOCCOCCN)c1]

By the same scheme used in the synthesis of DNP-amine, mMeOoNP-amine (1.28 g, 4.27 mmol) was acquired as a yellow oil (yield: 73%) using 2-fluoro-4-methoxy-1-nitrobenzene as a starting material.

¹H NMR (400 MHz, CD₃OD): δ 8.03 (d, 1H, J=9.2 Hz), 6.27 (d, 1H, J=2.8 Hz), 6.23 (dd, 1H, J=9.6, 2.8 Hz), 3.86 (s, 3H), 3.78 (t, 2H, J=5.2 Hz), 3.70-3.63 (m, 4H), 3.52 (t, 2H, J=5.2 Hz), 3.48 (t, 2H, J=5.2 Hz), 2.77 (t, 2H, J=5.2 Hz). ¹³C NMR (100 MHz CD₃OD): δ 167.6, 149.2, 129.8, 127.3, 106.2, 96.3, 73.6, 71.5, 71.3, 70.1, 56.3, 43.6, 42.2. HRMS (ESI) calcd for [M+Na]*, 322.13734; found, 322.13714 (Δ0.20 mmu).

### Synthesis of mCOOMeoNP-amine Methyl (3-((2-(2-(2-aminoethoxy)ethoxy)ethyl)amino)-4-nitrobenzoate

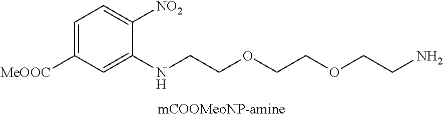 [SMILES: COC(=O)c1ccc([N+](=O)[O-])c(NCCOCCOCCN)c1]

By the same scheme used in the synthesis of DNP-amine, mCOOMeoNP-amine (1.11 g, 3.39 mmol) was acquired as an orange-colored oil (yield: 70%) using methyl 3-fluoro-4-nitrobenzoate as a starting material.

¹H NMR (400 MHz, CD₃OD): δ 7.98 (d. 1H, J=9.2 Hz), 7.37 (d, 1H, J=1.6 Hz), 7.01 (dd, 1H, J=8.8, 1.6 Hz), 3.88 (s, 3H), 3.76 (t, 2H, J=5.2 Hz), 3.71 (m, 4H), 3.51 (t, 2H, J=5.2 Hz), 3.44 (t, 2H, J=5.2 Hz), 2.77 (t, 2H, J=5.2 Hz). ¹³C NMR (100 MHz, CD₃OD): δ 166.7, 145.8, 137.3, 134.7, 127.7, 116.7, 115.9, 73.6, 71.5, 71.3, 69.9, 53.2, 43.5, 42.2. HRMS (ESI⁺) calcd. for [M+H]⁺, 328.15031; found, 328.15150 (Δ1.19 mmu).

### Synthesis of oDNP-Amine (N-(2-(2-(2-aminoethoxy) ethoxy)ethyl)-2,6-dinitroaniline)

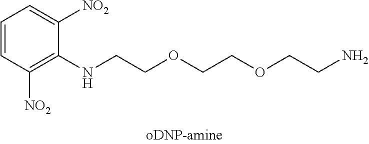 [SMILES: NCCOCCOCCNc1c([N+](=O)[O-])cccc1[N+](=O)[O-]]

By the same scheme used in the synthesis of DNP-amine, oDNP-amine (1.38 g, 4.40 mmol) was acquired as a brown oil (yield: 86%) using 2-chloro-1,3-dinitrobenzene as a starting material.

¹H NMR (400 MHz CD₃OD): δ 8.23 (d, 2H, J=8.0 Hz), 6.85 (t, 1H, J=8.0 Hz), 3.68-3.64 (m, 6H), 3.52 (t, 2H, J=5.2 Hz), 3.15 (t, 2H, J=5.2 Hz), 2.80 (t, 2H, J=5.2 Hz).

HRMS (ESI) calcd for [M+Na]⁺, 337.11186; found, 337.11280 (Δ 0.94 mmu).

### Synthesis of DCF3P-Amine (N-(2-(2-(2-aminoethoxy) ethoxy)ethyl)-2,4-bis(trifluoromethyl) aniline)

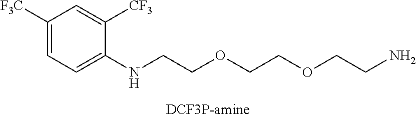 [SMILES: NCCOCCOCCNc1ccc(C(F)(F)F)cc1C(F)(F)F]

By the same scheme used in the synthesis of DNP-amine, DCF3P-amine (0.847 g, 2.35 mmol) was acquired as a colorless oil (yield: 55%) using 2-fluoro-1-nitro-4-(trifluoromethyl)benzene as a starting material.

¹H NMR (400 MHz, CD₃OD): δ 7.65-7.63 (m, 2H), 6.97 (d, 1H, J=8.4 Hz), 3.73 (t, 2H, J=5.2 Hz), 3.68-3.62 (m, 4H), 3.51 (t, 2H, J=5.2 Hz), 3.45 (t, 2H J=5.2 Hz), 2.78 (t, 2H, J=5.2 Hz). ¹³C NMR (100 MHz, CD₃OD): δ 149.7, 131.4-131.3 (m), 129.9-121.8 (m), 125.0 (m), 118.2 (q, J_{C-F}=33.4 Hz), 113.6 (q, J_{C-F}=30.2 Hz), 113.2, 73.6, 71.5, 71.4, 70.0, 43.8, 42.2. HRMS (ESI⁺) calcd. for [M+H]⁺, 361.13452; found, 361.13546 (Δ0.94 mmu).

### Synthesis of pCNoNP (N-(2-(2-(2-((4-cyano-2-nitrophenyl)amino)ethoxy)ethoxy)ethyl) acetamide)

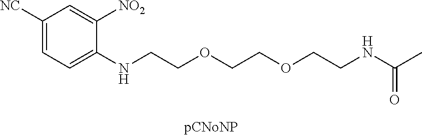 [SMILES: CC(=O)NCCOCCOCCNc1ccc(C#N)cc1[N+](=O)[O-]]

By the same scheme used in the synthesis of DNP, pCNoNP (220 mg, 0.653 mmol) was acquired as a yellow oil (yield: 96%) using pCNoNP-amine as a starting material.

¹H NMR (400 MHz, CDCl₃): δ 8.71 (br s, 1H), 8.50 (d, 1H, J=2.0 Hz), 7.62 (dd, 1H, J=9.2, 2.0 Hz), 6.94 (d, 1H, J=9.2 Hz), 6.22 (br s, 1H), 3.83 (t, 2H, J=5.2 Hz), 3.73-3.70 (m, 2H), 3.67-3.65 (m, 2H), 3.59-3.54 (m, 2H), 3.49-3.45 (m, 2H), 1.99 (s, 3H). ¹³C NMR (100 MHz, CDCl₃): δ 170.4, 147.2, 137.8, 132.3, 131.5, 118.0, 115.0, 98.3, 70.7, 70.2, 70.2, 68.3, 42.9, 39.4, 23.3. HRMS (ESI⁺) calcd. for [M+Na]⁺, 359.13259; found, 359.13431 (Δ1.49 mmu).

### Synthesis of 6SiR—NHS (2-(7-(dimethylamino)-3-(dimethylimino)-5,5-dimethyl-3,5-dihydrodibenzo[b,e]silin-10-yl)-4-(((2,5-dioxopyrrolidine-1-yl)oxy) carbonyl)benzoate)

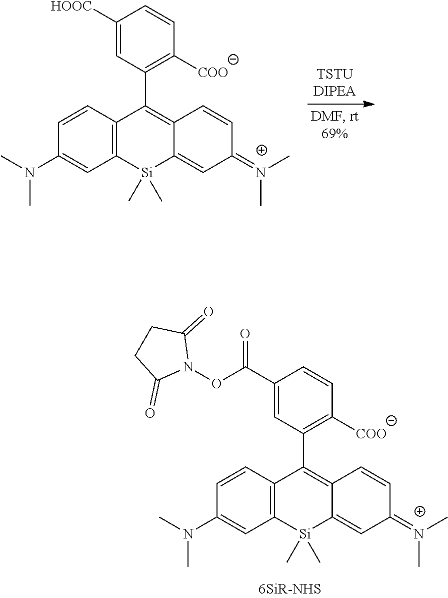

A reaction mixture in which SiR-carboxyl (G. Lukinavicius et al., A near-infrared fluorophore for live-cell super-resolution microscopy of cellular proteins. Nat Chem 5, 132-139 (2013)) (30 mg, 0.064 mmol), TSTU (23 mg, 0.076 mmol), and DIPEA (290 μL, 1.7 mmol) were dissolved in 0.5 mL of DMF was stirred for two hours at room temperature while shielded from light. After TFA (130 μL, 1.7 mmol) was added thereto, a coarse product was purified by HPLC, and 6SiR—NHS (25 mg, 0.044 mmol) was acquired as a green solid (yield: 69%).

¹H NMR (400 MHz DMSO-d₆): δ 8.36 (dd, 1H, J=8.0, 1.6 Hz), 8.20 (dd, 1H, J=8.0, 0.4 Hz), 7.943-7.938 (m, 1H), 7.20 (d, 2H, J=2.8 Hz), 6.75 (dd, 2H, J=8.8, 2.8 Hz), 2.99 (s, 12H), 2.95 (s, 4H), 0.68 (s, 3H), 0.58 (s, 3H). ¹³C NMR (100 MHz DMSO-d₆): δ 170.3, 169.4, 162.0, 156.4, 150.4, 141.5, 137.3, 132.1, 132.0, 131.4, 131.3, 129.2, 127.5, 126.6, 118.1, 115.2, 40.5, 26.3, 0.1, −0.9. HRMS (ESI⁻) calcd for C31H32N3O6Si [M+H]⁺, 570.20549; found, 570.20454 (Δ-0.95 mmu).

### Synthesis Example 16

### Synthesis of 6SiR-pCNoNP (4-((2-(2-(2-((4-cyano-2-nitrobenzyl)amino) ethoxy) ethoxy)ethyl)carbamoyl)-2-(7-(dimethylamino)-3-(dimethylamino)-5,5-dimethyl-3,5-dihydrodibenzo[b,e]silin-10-yl)benzoate)

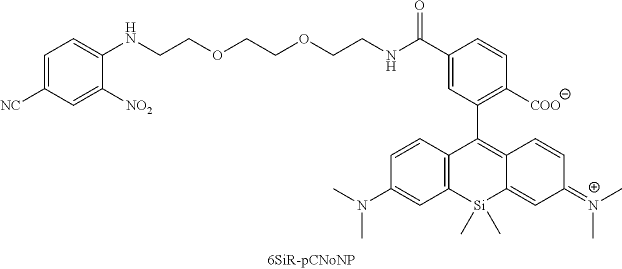

By the same scheme used in the synthesis of 6JF646-DNP, 6SiR-pCNoNP (2.4 mg, 0.0032 mmol) was acquired as a green solid (yield: 91%) using 6SiR—NHS and pCNoNP-amine as starting materials.

¹H NMR (400 MHz (CD₃)₂CO): δ 8.60 (br s, 1H), 8.43 (d, 1H, J=2.0 Hz), 8.09 (dd, 1H, J=8.0, 1.2 Hz), 7.98-7.96 (m, 2H), 7.77 (d, 1H, J=0.8 Hz), 7.71 (ddd, 1H, J=8.8, 2.0, 0.8 Hz), 7.13-7.11 (m, 3H), 6.75 (d, 2H, J=8.8 Hz), 6.65 (dd, 2H, J=8.8, 2.8 Hz), 3.73 (t, 2H, J=5.2 Hz), 3.61-3.60 (m, 6H), 5.54-3.49 (m, 4H), 2.97 (s, 12H), 0.69 (s, 3H), 0.55 (s, 3H). HRMS (ESI⁺) calcd. for [M+H]⁺, 749.31135; found, 749.31045 (Δ-0.90 mmu)

### Synthesis Example 17

### Synthesis of 6SiR-oNP (2-(7-(dimethylamino)-3-(diemthylimino)-5,5-dimethyl-3,5-dihydrobenzo[b,e]silin-10-yl)-4-(2-(2-(2-((2-(2-(2-(2-nitrophenyl)amino)ethoxy)ethoxy)ethyl)carbamoyl)benzoate)

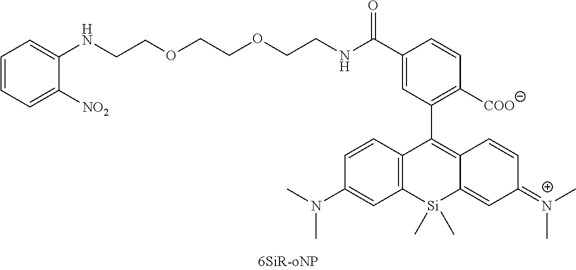

By the same scheme used in the synthesis of 6JF646-DNP, 6SiR-oNP (2.3 mg, 0.0032 mmol) was acquired as a greenish yellow solid (yield: 91%) using 6SiR—NHS and oNP-amine as starting materials.

¹H NMR (400 MHz, (CD₃)₂CO): δ 8.19 (br s, 1H), 8.10-8.05 (m, 2H), 7.98 (br s, 1H), 7.96-7.94 (dd, 1H, J=8.0, 0.4 Hz), 7.76 (d, 1H, J=0.4 Hz), 7.51-7.49 (m, 1H), 7.09 (d, 2H, J=2.8 Hz), 6.97 (d, 1H, J=8.0 Hz), 6.74 (d, 2H, J=8.8 Hz), 6.70-6.65 (m, 1H), 6.63 (dd, 2H, J=8.8, 2.8 Hz), 3.72 (t, 2H, J=5.2 Hz), 3.62-3.60 (m, 6H), 3.55-3.53 (m, 2H), 3.45-3.41 (m, 2H), 2.96 (s, 12H), 0.68 (s, 3H), 0.55 (s, 3H). HRMS (ESI⁺) calcd. for [M+H]⁺, 724.31610; found, 724.31450 (Δ-1.60mmu).

### Synthesis Example 18

### Synthesis of 6SiR-oDNP (2-(7-(dimethylamino)-3-(diemthylamino)-5,5-dimethyl-3,5-dihydrobenzo[b, e] silin-10-yl)-4-((2-(2-(2-((2,6-nitrophenyl)amino)ethoxy)ethoxy)ethyl)carbamoyl)benzoate)

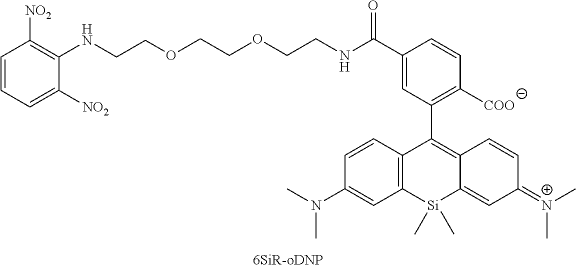

By the same scheme used in the synthesis of 6JF646-DNP, 6SiR-oDNP (2.6 mg, 0.0034 mmol) was acquired as a yellow solid (yield: 96%) using 6SiR—NHS and oDNP-amine as starting materials.

¹H NMR (400 MHz, (CD₃)₂CO): δ 8.52 (br s, 1H), 8.22 (d, 2H, J=8.4 Hz), 8.07 (dd, 1H, J=8.0, 1.2 Hz), 7.96-7.94 (m, 2H), 7.75 (d, 1H, J=0.8 Hz), 7.09 (d, 2H, J=2.8 Hz), 6.89 (t, 1H, J=8.0 Hz), 6.75 (d, 2H, J=8.8 Hz), 6.64 (dd, 2H, J=9.2, 2.8 Hz), 3.65 (t, 2H, J=2.8 Hz), 3.60-3.58 (m, 6H), 3.55-3.51 (m, 2H), 3.09-3.05 (m, 2H), 2.96 (s, 12H), 0.69 (s, 3H), 0.55 (s, 3H). HRMS (ESI⁴) calcd. for [M+H]⁺, 769.30118; found, 769.30113 (Δ−0.05 mmu).

### Synthesis Example 19

### Synthesis of 6SiR-Linker (2-(7-(dimethylamino)-3-(diemthyliminio)-5,5-dimethyl-3,5-dihydrodibenzo[b,e]silin-10-yl)-4-((2-(2-methoxyethoxy)ethyl)carbamoyl)benzoate)

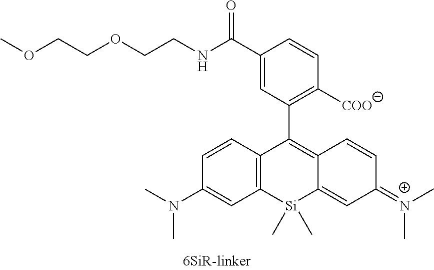

By the same scheme used in the synthesis of 6JF646-DNP, 6SiR-linker (0.8 mg, 0.0014 mmol) was acquired as a green solid (yield: 40%) using 6SiR—NHS and 2-(2-methoxyethoxy) ethane-1-amine as starting materials.

¹H NMR (400 MHz, (CD₃)₂CO): δ 8.11 (dd, 1H, J=8.0, 1.2 Hz), 8.02 (br s, 1H), 7.99 (d, 1H, J=8.0 Hz), 7.75 (s, 1H), 7.11 (d, 2H, J=2.8 Hz), 6.75 (d, 2H, J=9.2 Hz), 6.65 (dd, 2H, J=8.8, 2.8 Hz), 3.59-3.51 (m, 6H), 3.43-3.41 (m, 2H), 3.20 (s, 3H), 2.97 (s, 12H), 0.68 (s, 3H), 0.56 (s, 3H). HRMS (ESI=) calcd. for [M+H]⁺, 574.27317; found, 574.27444 (Δ1.27 mmu).

### Synthesis Example 20

### Synthesis of 6SiR-mCNoNP (4-((2-(2-(2-((5-cyano-2-nitrophenyl)amino)ethoxy)ethoxy)ethyl)carbamoyl)-2-(7-(dimethylamino)-3-(dimethylimino)-5,5-dimethyl-3,5-dihyxodibenzo[b,e]silin-10-yl)benzoate)

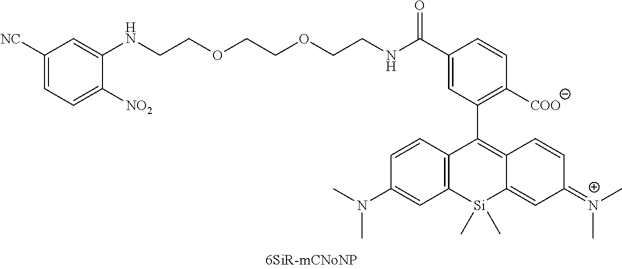

By the same scheme used in the synthesis of 6JF646-DNP, 6SiR-mCNoNP (2.0 mg, 0.0027 mmol) was acquired as a yellow solid (yield: 76%) using 6SiR—NHS and mCNoNP-amine as starting materials.

¹H NMR (400 MHz, (CD₃)₂CO): δ 8.25 (br s, 1H), 8.19 (d, 1H, J=8.8 Hz), 8.08 (d, 1H, J=8.0 Hz), 7.96-7.94 (m, 2H), 7.75 (s, 1H), 7.48 (s, 1H), 7.10 (d, 2H, J=2.4 Hz), 6.97 (d, 1H, J=8.8 Hz), 6.76 (d, 2H, J=9.2 Hz), 6.64 (dd, 2H, J=8.8, 2.8 Hz), 3.75 (t, 2H, J=5.2 Hz), 3.61-3.60 (m, 6H), 3.55-3.51 (m, 4H), 2.96 (s, 12H), 0.69 (s, 3H), 0.55 (s, 3H). HRMS (ESI⁺) calcd. for [M+H]⁺, 749.31135; found, 749.31022 (Δ-1.13 mmu).

### Synthesis Example 21

### Synthesis of 6SiR-pCF3oNP (2-(7-(dimethylamino)-3-(dimethylimino)-5,5-dimethyl-3,5-dihydrodibenzo[b,e]silin-10-yl)-4-((2-(2-(2-((2-nitro-4-(trifluoromethyl)phenyl)amino)ethoxy)ethoxy)ethyl)carbamoyl)benzoate)

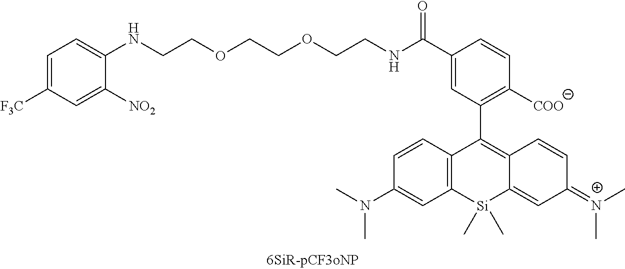

By the same scheme used in the synthesis of 6JF646-DNP, 6SiR-pCF3oNP (2.0 mg, 0.0025 mmol) was acquired as a yellowish green solid (yield: 72%) using 6SiR—NHS and pCF3oNP-amine as starting materials.

¹H NMR (400 MHz, (CD₃)₂CO): δ 8.50 (br s, 1H), 8.36 (d, 1H, J=1.2 Hz), 8.08 (dd, 1H, J=8.0, 1.6 Hz), 7.98 (br s, 1H), 7.95 (dd, 1H, J=8.0, 0.4 Hz), 7.76 (s, 1H), 7.73 (dd, 1H, J=9.2, 2.4 Hz), 7.17 (d, 1H, J=9.2 Hz), 7.10 (d, 2H, J=2.8 Hz), 6.75 (d, 2H, J=9.2 Hz), 6.64 (dd, 2H, J=8.8, 2.8 Hz), 3.74 (t, 2H, J=5.2 Hz), 3.64-3.60 (m, 6H), 3.59-3.48 (m, 4H), 2.95 (s, 12H), 0.68 (s, 3H), 0.55 (s, 3H). HRMS (ESI⁺) calcd. for [M+H]⁺, 792.303349; found, 792.30515 (Δ1.66 mmu).

### Synthesis Example 22

### Synthesis of 6SiR-pCOOMeoNP (2-(7-(dimethylamino)-3-(dimethylimino)-5,5-dimethyl-3,5-dihydrodibenzo[b,e]silin-10-yl)-4-((2-(2-(2-((4-(methoxycarbonyl)-2-nitrophenyl)amino)ethoxy)ethoxy)ethyl)carbamoyl)benzoate)

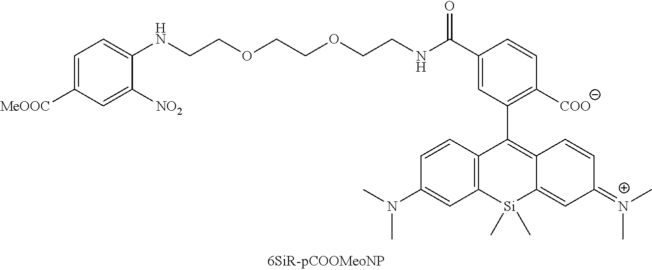

By the same scheme used in the synthesis of 6JF646-DNP, 6SiR-pCOOMeoNP (5.0 mg, 0.0064 mmol) was acquired as a green solid (yield: 91%) using 6SiR—NHS and pCOOMeoNP-amine as starting materials.

¹H NMR (400 MHz, (CD₃)₂CO): δ 8.69 (d, 1H, J=2.4 Hz), 8.56 (br s, 1H), 8.11 (dd, 1H, J=8.0, 1.2 Hz), 8.01-7.96 (m, 3H), 7.82 (br s, 2H), 7.42 (br s, 2H), 7.07 (d, 2H, J=9.2 Hz), 6.91-6.86 (m, 3H), 3.86 (s, 3H), 3.75 (t, 2H, J=5.2 Hz), 3.63-3.60 (m, 6H), 3.54-3.52 (m, 4H), 3.01 (s, 12H), 0.72 (s, 3H), 0.58 (s, 3H). HRMS (ESI⁺) calcd. for [M+H]⁺, 782.32158; found, 782.32129 (Δ−0.29 mmu).

### Synthesis Example 23

### Synthesis of 6SiR-pBroNP (4-((2-(2-(2-((4-bromo-2-nitrophenyl)amino) ethoxy) ethoxy)ethyl)carbamoyl)-2-(7-(dimethylamino)-3-(dimethylimino)-5,5-dimethyl-3,5-dihydrodibenzo[b,e]silin-10-yl)benzoate)

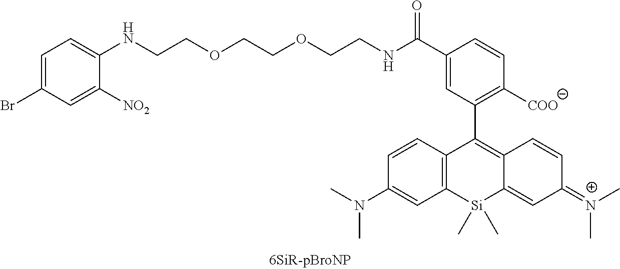

By the same scheme used in the synthesis of 6JF646-DNP, 6SiR-pBroNP (2.4 mg, 0.0030 mmol) was acquired as a green solid (yield: 85%) using 6SiR—NHS and pBroNP-amine as starting materials.

¹H NMR (400 MHz, (CD₃)₂CO): δ 8.22 (br s, 1H), 8.16 (d, 1H, J=2.4 Hz), 8.10 (dd, 1H, J=8.0, 1.2 Hz), 7.98-7.96 (m, 2H), 7.78 (s, 1H), 7.57 (dd, 1H, J=9.2, 2.4 Hz), 7.20 (br s, 2H), 6.98 (d, 1H, J=9.2 Hz), 6.79 (d, 2H, J=8.8 Hz), 6.73-6.71 (m, 2H), 3.71 (t, 2H, J=5.2 Hz), 3.62-3.59 (m, 6H), 3.55-3.51 (m, 2H), 3.45-3.41 (m, 2H), 2.98 (s, 12H), 0.70 (s, 3H), 0.56 (s, 3H). HRMS (ESI⁺) calcd. for [M+H]⁺, 802.22661; found, 802.22786 (A 1.25 mmu).

### Synthesis Example 24

### Synthesis of 6SiR-pCOONHMeoNP (2-(7-(dimethylamino)-3-(dimethylimino)-5,5-dimethyl-3,5-dihydrodibenzo[b,e] silin-10-yl)-4-((2-(2-(2-((4-(methylcarbamoyl)-2-nitrophenyl)amino)ethoxy)ethoxy)ethyl)carbamoyl)benzoate)

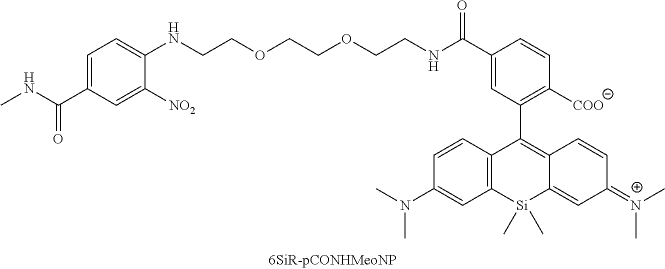

6SiR-pCOOMeoNP (2.4 mg, 0.0031 mmol) was dissolved in 0.5 mL of THF, 0.5 mL of water was added thereto, and the mixture was then stirred at room temperature while shielded from light. While reaction progress was confirmed by TLC, a 1 M sodium hydroxide aqueous solution was dropped therein 11 μL at a time. After dropping a total of 55 μL of the 1 M sodium hydroxide aqueous solution, 60 μL of 1 M hydrochloric acid was added to the reaction solution, and reaction was stopped. The reaction mixture was extracted with dichloromethane and then washed with a saturate saline solution, and dehydration with sodium sulfate, filtration, and concentration were performed. A coarse product was purified by HPLC, and an intermediate (1.0 mg, 0.0013 mmol) was acquired as a green solid. A reaction mixture in which the intermediate, TSTU (0.5 mg, 0.0016 mmol), DIPEA (5.9 μL, 0.034 mmol) were dissolved in 0.5 mL of DMF was stirred for one hour at room temperature while shielded from light. A 40% methylamine aqueous solution (0.4 μL, 0.0049 mmol) was furthermore added, and the mixture was stirred for 15 minutes. A coarse product was purified by HPLC, and 6SiR-pCONHMeoNP (1.0 mg, 0.0013 mmol) was acquired as a green solid (yield of the two reactions: 42%).

¹H NMR (400 MHz, (CD₃)₂CO): δ 8.63 (d, 1H, J=2.0 Hz), 8.41 (br s, 1H), 8.09 (d, 1H, J=8.0 Hz), 8.00-7.99 (m, 2H), 7.95 (d, 1H, J=7.6 Hz), 7.76 (m, 2H), 7.09 (d, 2H, J=2.4 Hz), 7.04 (d, 1H, J=9.2 Hz), 6.74 (d, 2H, J=8.8 Hz), 6.63 (dd, 2H, J=9.2, 2.4 Hz), 3.72 (t, 1H, J=5.2 Hz), 3.61-3.59 (m, 6H), 3.55-3.47 (m, 4H), 2.95 (s, 12H), 2.87 (d, 3H, J=4.4 Hz), 0.68 (s, 3H), 0.55 (s, 3H). HRMS (ESI⁺) calcd. for [M+H]⁺, 781.33757; found, 781.33757 (A 0.00 mmu).

### Synthesis Example 25

### Synthesis of 6SiR-mCOOMeoNP (2-(7-(dimethylamino)-3-(dimethylimino)-5,5-dimethyl-3,5-dihydrodibenzo[b,e]silin-10-yl)-4-((2-(2-(2-((5-(methoxycarbonyl)-2-nitrophenyl)amino)ethoxy)ethoxy)ethyl)carbamoyl)benzoate)

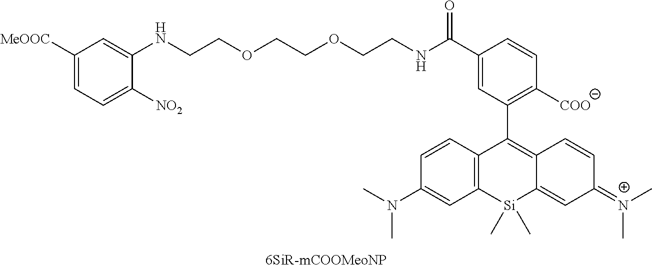

By the same scheme used in the synthesis of 6JF646-DNP, 6SiR-mCOOMeoNP (3.0 mg, 0.0038 mmol) was acquired as a yellowish green solid (yield: 55%) using 6SiR—NHS and mCOOMeoNP-amine as starting materials.

¹H NMR (400 MHz, (CD₃)₂CO): δ 8.15-8.13 (m, 2H), 8.00 (dd, 1H, J=8.0, 1.6 Hz), 7.90-7.88 (m, 2H), 7.71-7.70 (m, 1H), 7.50 (d, 1H, J=1.6 Hz), 7.20 (dd, 1H, J=8.8, 1.6 Hz), 7.09 (d, 2H, J=2.8 Hz), 6.78 (d, 2H, J=9.2 Hz), 6.65 (dd, 2H, J=8.8, 2.8 Hz), 3.92 (s, 3H), 3.72 (t, 2H, J=5.2 Hz), 3.62-3.60 (m, 6H), 3.54-3.49 (m, 2H), 3.39-3.35 (m, 2H), 2.95 (s, 12H), 0.69 (s, 3H), 0.54 (s, 3H). HRMS (ESI ⁺) calcd. for [M+H]⁺, 782.32158; found, 782.32292 (Δ1.34 mmu).

### Synthesis Example 26

### Synthesis of 6SiR-mCF3oNP (2-(7-(dimethylamino)-3-(dimethylimino)-5,5-dimethyl-3,5-dihydrodibenzo[b,e]silin-10-yl)-4-((2-(2-(2-((2-nitro-5-(trifluoromethyl)phenyl)amino)ethoxy)ethoxy)ethyl) carbamoyl)benzoate)

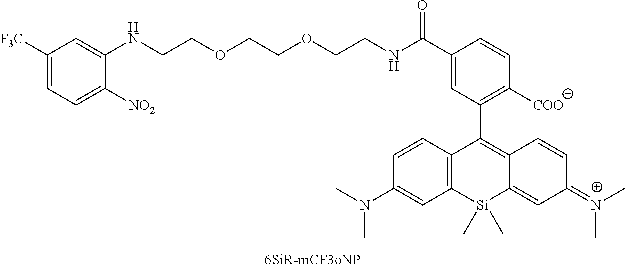

By the same scheme used in the synthesis of 6JF646-DNP, 6SiR-mCF3oNP (2.0 mg, 0.0025 mmol) was acquired as a yellowish green solid (yield: 72%) using 6SiR—NHS and mCF3oNP-amine as starting materials.

¹H NMR (400 MHz, (CD₃)₂CO): δ 8.31 (br s, 1H), 8.25 (dd, 1H, J=8.8, 0.4 Hz), 8.07 (dd, 1H, J=8.0, 1.6 Hz), 7.95-7.93 (m, 2H), 7.75-7.74 (m, 1H), 7.33 (s, 1H), 7.09 (d, 2H, J=3.2 Hz), 6.94 (dd, 1H, J=8.8, 1.6 Hz), 6.75 (d, 2H, J=9.2 Hz), 6.64 (dd, 2H, J=9.2, 3.2 Hz), 3.75 (t, 2H, J=5.2 Hz), 3.62-3.59 (m, 6H), 3.57-3.52 (m, 4H), 2.95 (s, 12H), 0.69 (s, 3H), 0.55 (s, 3H). HRMS (ESI⁺) calcd. for [M+H]⁺, 792.30349; found, 795.30297 (Δ−0.52 mmu).

### Synthesis Example 27

### Synthesis of 6SiR-pSO2MeoNP (2-(7-(dimethylamino)-3-(dimethylimino)-5,5-dimethyl-3,5-dihydrodibenzo[b,e]silin-10-yl)-4-((2-(2-(2-((4-(methylsulfonyl)-2-nitrophenyl)amino)ethoxy)ethoxy)ethyl) carbamoyl)benzoate)

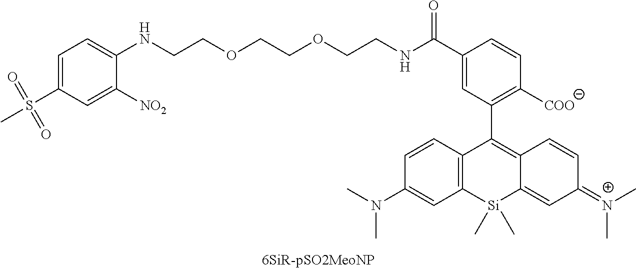

By the same scheme used in the synthesis of 6JF646-DNP, 6SiR-pSO2MeoNP (1.7 mg, 0.0018 mmol) was acquired as a yellowish green solid (yield: 60%) using 6SiR—NHS and pSO2MeoNP-amine as starting materials.

¹H NMR (400 MHz, (CD₃)₂CO): δ 8.63 (br s, 1H), 8.58 (d, 1H, J=2.4 Hz), 8.08 (dd, 1H, J=8.0, 1.6 Hz), 7.97-7.95 (m, 2H), 7.91 (dd, 1H, J=9.2, 2.4 Hz), 7.77 (d, 1H, J=0.4 Hz), 7.21-7.18 (m, 3H), 6.81-6.73 (m, 4H), 3.76 (t, 2H, J=5.2 Hz), 3.63-3.60 (m, 6H), 3.56-3.51 (m, 4H), 3.10 (s, 3H), 2.98 (s, 12H), 0.70 (s, 3H), 0.56 (s, 3H). HRMS (ESI⁺) calcd. for [M+H]⁺, 824.27560; found, 824.27660 (Δ1.00 mmu).

### Synthesis Example 28

### Synthesis of 6SiR-pCloNP (4-((2-(2-(2-((4-chloro-2-nitrophenyl)amino)ethoxy)ethoxy)ethyl)carbamoyl)-2-(7-(dimethylamino)-3-(dimethylimino)-5,5-dimeth yl-3,5-dihydrodibenzo[b,e]silin-10-yl)benzoate)

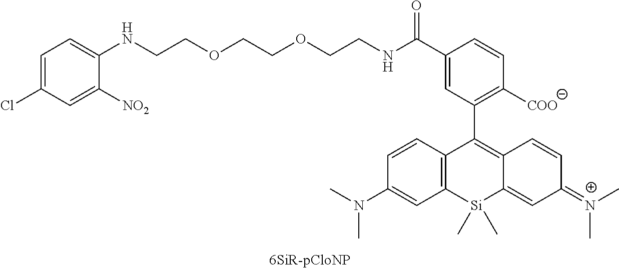

By the same scheme used in the synthesis of 6JF646-DNP, 6SiR-pCloNP (2.4 mg, 0.0030 mmol) was acquired as a green solid (yield: 85%) using 6SiR—NHS and pCloNP-amine as starting materials.

¹H NMR (400 MHz, (CD₃)₂CO): δ 8.25 (br s, 1H), 8.07-8.05 (m, 2H), 7.95-7.93 (m, 2H), 7.743-7.738 (m, 1H), 7.09 (d, 2H, J=3.2 Hz), 7.05 (d, 1H, J=2.0 Hz), 6.76 (d, 2H, J=8.8 Hz), 6.68-6.62 (m, 3H), 3.72 (t, 2H, J=5.2 Hz), 3.63-3.60 (m, 6H), 3.55-3.51 (m, 2H), 3.44-3.40 (m, 2H), 2.96 (s, 12H), 0.70 (s, 3H), 0.55 (s, 3H). HRMS (ESI⁺) calcd. for [M+H]⁺, 758.27713; found, 758.28038 (Δ3.25 mmu).

### Synthesis Example 29

### Synthesis of 6SiR-LC-oNP (2-(7-(dimethylamino)-3-(dimethylimino)-5,5-dimethyl-3,5-dihydrodibenzo[b,e]silin-10-yl)-4-((1-((2-nitrophenyl)amino)-10-oxo-3,6,13,16,19-pentaoxa-9-azahenicosan-21-yl)carbamoyl)benozate

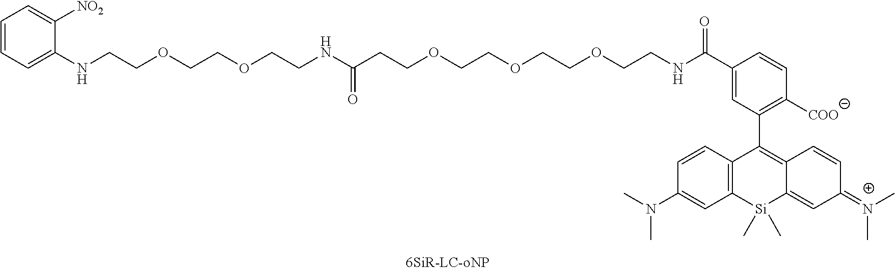

By the same scheme used in the synthesis of 6JF646-DNP, 6SiR-LC-oNP (1.7 mg, 0.0018 mmol) was acquired as a green solid (yield: 71%) using 6SiR—NHS and LC-oNP-amine as starting materials.

¹H NMR (400 MHz, (CD₃)₂CO): δ 8.22 (br s, 1H), 8.15-8.09 (m, 3H), 8.00-7.97 (dd, 1H, J=8.0, 0.8 Hz), 7.80-7.79 (m, 1H), 7.53-7.49 (m, 1H), 7.10 (d, 2H, J=2.8 Hz), 7.07-7.04 (m, 2H), 6.75 (d, 2H, J=9.2 Hz), 6.71-6.67 (m, 1H), 6.65 (dd, 2H, J=8.8, 2.8 Hz), 3.77 (t, 2H, J=5.2 Hz), 3.65-3.43 (m, 22H), 3.31-3.27 (m, 2H), 2.97 (s, 12H), 2.29 (t, 2H, J=6.0 Hz), 0.69 (s, 3H), 0.55 (s, 3H). HRMS (ESI⁺) calcd. for [M+H]⁺, 949.41380; found, 949.41383 (Δ0.00 mmu)

### Synthesis Example 30

### Synthesis of 6SiR-LC-pCF3oNP (2-(7-(dimethylamino)-3-(dimethylimino)-5,5-dimethyl-3,5-dihydrodibenzo[b,e]silin-10-yl)-4-((1-((2-nitro-4-(trifluoromethyl)phenyl)amino)-10-oxo-3,6,13,16,19-pentaoxa-9-azahenicosan-21-yl)carbamoyl)benozate)

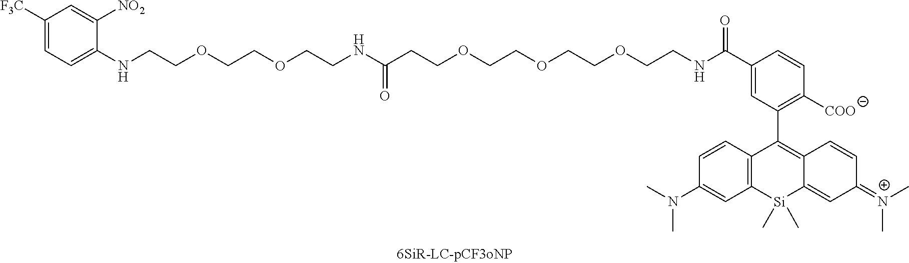

By the same scheme used in the synthesis of 6JF646-DNP, 6SiR-LC-pCF3oNP (1.8 mg, 0.0018 mmol) was acquired as a green solid (yield: 70%) using 6SiR—NHS and LC-pCF3oNP-amine as starting materials.

¹H NMR (400 MHz, (CD₃)₂CO): δ 8.55 (br s, 1H), 8.39 (d, 1H, J=1.2 Hz), 8.15-8.11 (m, 2H), 7.98 (dd, 1H, J=8.0, 0.8 Hz), 7.80-7.79 (m, 1H), 7.56 (dd, 1H, J=9.2, 2.4 Hz), 7.28 (d, 1H, J=9.2 Hz), 7.11-7.08 (m, 3H), 6.75 (d, 2H, J=9.2 Hz), 6.65 (dd, 2H, J=9.2, 2.8 Hz), 3.80 (t, 2H, J=5.2 Hz), 3.66-3.42 (m, 22H), 3.31-3.27 (m, 2H), 2.96 (s, 12H), 2.29 (t, 2H, J=6.0 Hz), 0.69 (s, 3H), 0.55 (s, 3H). HRMS (ESI⁺) calcd. for [M+H]⁺, 1017.40119; found, 1017.39989 (Δ−1.30 mmu).

### Synthesis Example 31

### Synthesis of 6SiR-LC-pCOOMeoNP (2-(7-(dimethylamino)-3-(dimethylimino)-5,5-dimethyl-3,5-dihydrodibenzo[b,e]silin-10-yl)-4-((1-((4-methoxycarbonyl)-2-nitrophenyl)amino)-10-oxo-3,6,13,16,19-pentaoxa-9-azahenicosan-21-yl)carbamoyl)benozate)

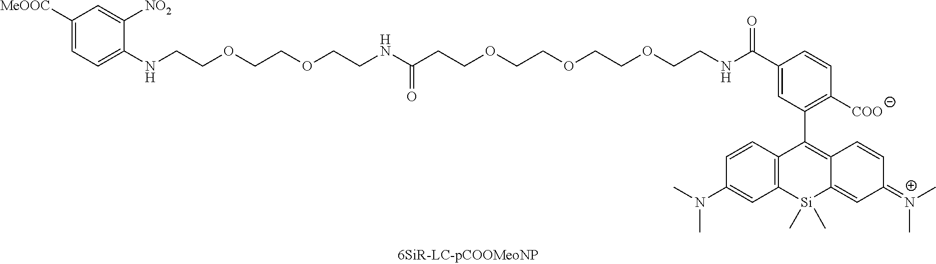

By the same scheme used in the synthesis of 6JF646-DNP, 6SiR-LC-pCOOMeoNP (1.3 mg, 0.0013 mmol) was acquired as a green solid (yield: 51%) using 6SiR—NHS and LC-pCOOMeoNP-amine as starting materials.

¹H NMR (400 MHz, (CD₃)₂CO): δ 8.75 (d, 1H, J=5.2 Hz), 8.60 (br s, 1H), 8.15-8.03 (m, 2H), 8.03-8.01 (m, 1H), 7.99-7.97 (dd, 1H, J=7.6, 0.8 Hz), 7.798-7.795 (m, 1H), 7.15 (d, 1H, J=9.2 Hz), 7.10 (d, 2H, J=9.2 Hz), 7.07 (br s, 1H), 6.75 (d, 2H, J=9.2 Hz), 6.65 (dd, 2H, J=9.2, 2.8 Hz), 3.86 (s, 3H), 3.80 (t, 2H, J=5.2 Hz), 3.65-3.42 (m, 22H), 3.32-3.27 (m, 2H), 2.97 (s, 12H), 2.28 (t, 2H, J=6.0 Hz), 0.69 (s, 3H), 0.55 (s, 3H). HRMS (ESI⁺) calcd. for [M+H]⁺, 1007.41928; found, 1007.41634 (Δ−2.94mmu)

### Synthesis Example 32

### Synthesis of 6SiR-pMeoNP (2-(7-(dimethylamino)-3-(dimethylimino)-5,5-dimethyl-3,5-dihydrodibenzo[b,e]silin-10-yl)-4-((2-(2-(2-((4-methyl-2-nitrophenyl)amino)ethoxy)ethoxy)ethyl)carbamoyl)benzoate)

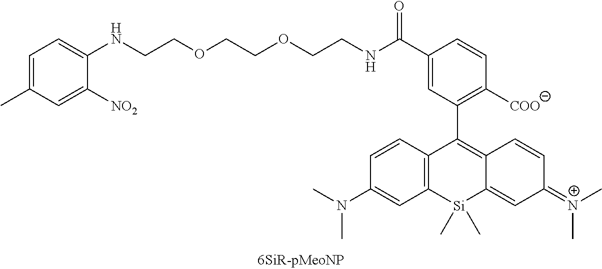

By the same scheme used in the synthesis of 6JF646-DNP, 6SiR-pMeoNP (1.7 mg, 0.0023 mmol) was acquired as a green solid (yield: 89%) using 6SiR—NHS and pMeoNP-amine as starting materials.

¹H NMR (400 MHz, (CD₃)₂CO): δ 8.09-8.07 (m, 2H), 7.95-7.93 (m, 2H), 7.87 (s, 1H), 7.78 (s, 1H), 3.34 (dd, 1H, J=8.8, 1.6 Hz), 7.09 (d, 2H, J=2.8 Hz), 6.89 (d, 1H, J=8.8 Hz), 6.74 (d, 2H, J=8.8 Hz), 6.63 (dd, 1H, J=8.8, 2.8 Hz), 3.72 (t, 2H, J=5.2 Hz), 3.62-3.60 (m, 6H), 3.55-3.51 (m, 2H), 3.42-3.38 (m, 2H), 2.95 (s, 12H), 0.68 (s, 3H), 0.55 (s, 3H). HRMS (ESI⁺) calcd. for [M+H]⁺, 738.33175; found, 738.33238 (Δ0.63 mmu).

### Synthesis Example 33

### Synthesis of 6SiR-pMeOoNP (2-(7-(dimethylamino)-3-(dimethylimino)-5,5-dimethyl-3,5-dihydrodibenzo[b,e]silin-10-yl)-4-((2-(2-(2-((4-methoxy-2-nitrophenyl)amino)ethoxy)ethoxy)ethyl)carbamoyl)benzoate)

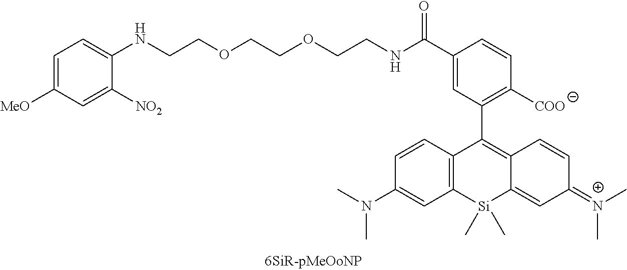

By the same scheme used in the synthesis of 6JF646-DNP, 6SiR-pMeOoNP (1.3 mg, 0.0017 mmol) was acquired as a green solid (yield: 67%) using 6SiR—NHS and pMeOoNP-amine as starting materials.

¹H NMR (400 MHz, (CD₃)₂CO): δ 8.09-8.07 (m, 2H), 7.96-7.93 (m, 2H), 7.76 (s, 1H), 7.53 (d, 1H, J=3.2 Hz), 7.20 (dd, 1H, J=9.6, 2.8 Hz), 7.09 (d, 2H, J=2.8 Hz), 7.01-6.96 (m, 1H), 6.74 (d, 2H, J=8.8 Hz), 6.63 (dd, 2H, J=9.2, 3.2 Hz), 3.79 (s, 3H), 3.70 (t, 2H, J=5.2 Hz), 3.62-3.61 (m, 6H), 3.55-3.51 (m, 2H), 3.43-3.39 (m, 2H), 2.96 (s, 12H), 0.68 (s, 3H), 0.55 (s, 3H). HRMS (ESI⁺) calcd. for [M+H]⁺, 754.32667; found, 754.32777 (Δ1.10 mmu).

### Synthesis Example 34

### Synthesis of 6SiR-DCF3P (4-((2-(2-(2-((2,4-bis(trifluoromethyl)phenyl)amino)ethoxy)ethoxy)ethyl)carbamoyl)-2-(7-(dimethylamino)-3-(dimethylimino)-5,5-dimethyl-3,5-dihydrodibenzo[b,e]silin-10-yl)benzoate)

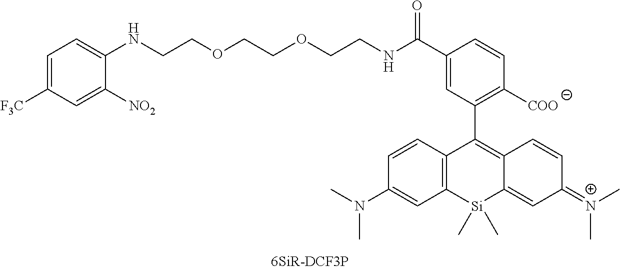

By the same scheme used in the synthesis of 6JF646-DNP, 6SiR-DCF3P (2.1 mg, 0.0026 mmol) was acquired as a pale green solid (yield: 73%) using 6SiR—NHS and DCF3P-amine as starting materials.

¹H NMR (400 MHz, (CD₃)₂CO): δ 8.10 (d, 1H, J=8.0 Hz). 8.00-7.94 (m, 2H), 7.76 (s, 1H), 7.70-7.68 (m, 2H), 7.10 (d, 2H, 2.8 Hz), 6.99 (d, 1H, J=9.2 Hz), 6.75 (d, 2H, J=9.2 Hz), 6.64 (dd, 2H, J=8.8, 2.8 Hz), 3.68 (t, 2H, J=5.6 Hz), 3.61-3.59 (m, 6H), 3.55-3.52 (m, 2H), 3.42-3.38 (m, 2H), 2.96 (s, 12H), 0.68 (s, 3H), 0.55 (s, 3H). HRMS (ESI⁺) calcd. for [M+H]⁺, 815.30579; found, 815.30687 (Δ1.08 mmu).

### Synthesis Example 35

### Synthesis of 6SiR-pCF30oNP (2-(7-(dimethylamino)-3-(dimethylimino)-5,5-dimethyl-3,5-dihydrodibenzo[b,e]silin-10-yl)-4-((2-(2-(2-((2-nitro-4-(trifluoromethoxy)phenyl)amino)ethoxy)ethoxy)ethyl)carbamoyl)benzoate)

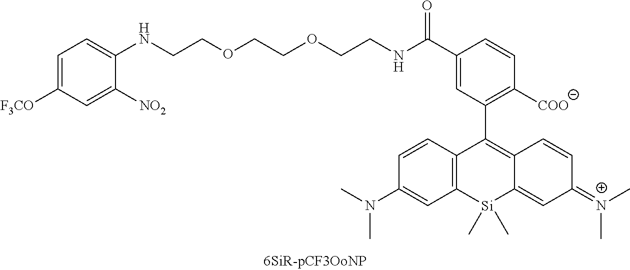

By the same scheme used in the synthesis of 6JF646-DNP, 6SiR-pCF30oNP (1.7 mg, 0.0020 mmol) was acquired as a green solid (yield: 58%) using 6SiR—NHS and pCF30oNP-amine as starting materials.

¹H NMR (400 MHz, (CD₃)₂CO): δ 8.26 (br s, 1H), 8.87 (dd, 1H, J=8.0, 1.2 Hz), 8.05 (d, 1H, J=2.8 Hz), 7.96-7.94 (m, 2H), 7.76 (s, 1H), 7.50 (dd, 1H, J=9.6, 2.8 Hz), 7.12-7.09 (m, 3H), 6.75 (d, 2H, J=8.8 Hz), 6.64 (dd, 2H, J=8.8, 2.8 Hz), 3.73 (t, 2H, J=5.2 Hz), 3.63-3.60 (m, 6H), 3.55-3.51 (m, 2H), 3.48-3.44 (m, 2H), 2.96 (s, 12H), 0.69 (s, 3H), 0.55 (s, 3H). HRMS (ESI⁺) calcd. for [M+H]⁺, 808.29840; found, 808.29775 (Δ−0.65 mmu).

### Synthesis Example 36

### Synthesis of 6SiR720-NHS (2-(1,2,2,4,8,10,10,11,13,13-decamethyl-2,10,11,13-tetrahydrosilino[3,2-g:5,6-g′]diquinoline-1-ium-6-yl)-4-(((2,5-dioxopyrrolidin-1-yl)oxy)carbonyl)benzoate)

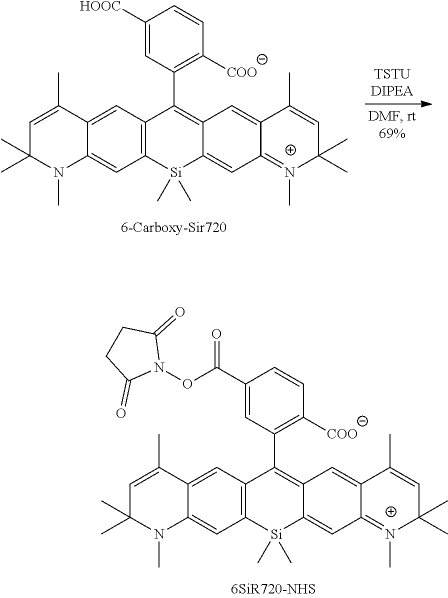

By the same scheme used in the synthesis of 6SiR—NHS, 6SiR720-NHS (11 mg, 0.016 mmol) was acquired as a green solid (yield: 69%) using 6SiR—NHS and 6-carboxy-SiR as starting materials. 1H NMR (400 MHz, (CD₃)₂CO): δ 8.41 (dd, 1H, J=8.0, 1.2 Hz), 8.34 (d, 1H, J=8.0 Hz), 8.03 (d, 1H, J=1.2 Hz), 7.14 (s, 2H), 6.64 (s, 2H), 5.472-5.470 (m, 2H), 3.14 (s, 6H), 2.95 (s, 4H), 1.63 (s, 3H), 1.62 (s, 3H), 1.42 (s, 6H), 1.40 (s, 6H), 0.66 (s, 3H), 0.58 (s, 3H). HRMS (ESI⁺) calcd. for [M+H]⁺, 702.29939; found, 702.30020 (Δ0.81 mmu).

### Synthesis Example 37

### Synthesis of 6SiR720-DNP (2-(1,2,2,4,8,10,10,11,13,13-decamethyl-2,10,11,13-tetrahydrosilino[3,2-g:5,6-g′]diquinoline-1-ium-6-yl)-4-((2-(2-(2-((2,4-dinitrophenyl)amino)ethoxy)ethoxy)ethyl)carbamoyl)benzoate)

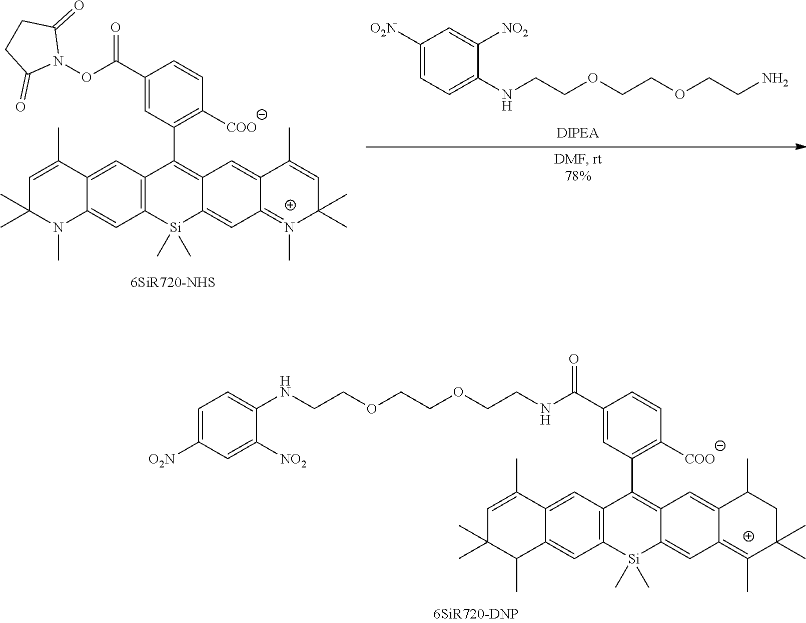

By the same scheme used in the synthesis of 6JF646-DNP, 6SiR720-DNP (7.0 mg, 0.0078 mmol) was acquired as a green solid (yield: 78%) using 6SiR720-NHS and DNP-amine as starting materials.

¹H NMR (400 MHz, (CD₃)₂CO): δ 8.91 (d, 1H, J=2.8 Hz), 8.79 (br s, 1H), 8.23 (dd, 1H, J=9.6, 2.8 Hz), 8.11 (d, 1H, J=8.0 Hz), 8.01 (d, 1H, J=8.0 Hz), 7.95 (br t, 1H, J=5.2 Hz), 7.82 (s, 1H), 7.14 (d, 1H, J=9.6 Hz), 6.88 (s, 2H), 6.55 (s, 2H), 5.34 (s, 2H), 3.76 (t, 2H, J=5.2 Hz), 3.62-3.51 (m, 10H), 2.93 (s, 6H), 1.61 (s, 6H), 1.32 (s, 6H), 1.29 (s, 6H), 0.68 (s, 3H), 0.54 (s, 3H) ¹³C NMR (100 MHz, (CD₃) ₂CO): δ 169.9, 166.2, 149.5, 145.8, 141.0, 136.6, 132.03, 131.98, 131.1, 130.7, 129.6, 129.0, 127.7, 126.6, 124.44, 124.40, 124.3, 123.3, 116.0, 115.8, 71.2, 70.9, 70.2, 69.3, 57.7, 43.9, 40.7, 31.3, 28.4, 27.8, 18.1, 0.24, −1.1. HRMS (ESI⁺) calcd. for [M+H]⁺, 901.39508; found, 901.39525 (Δ0.17 mmu).

### Synthesis Example 38

### Synthesis of 6SiR720-pCF3oNP (2-(1,2,2,4,8,10,10,11,13,13-decamethyl-2,10,11,13-tetrahydrosilino[3,2-g:5,6-g′ ] diquinoline-1-ium-6-yl)-4-((2-(2-(2-((2-nitro-4-(trifluoromethyl)phenyl)amino)ethoxy)ethoxy)ethyl)carbamoyl)benzoate)

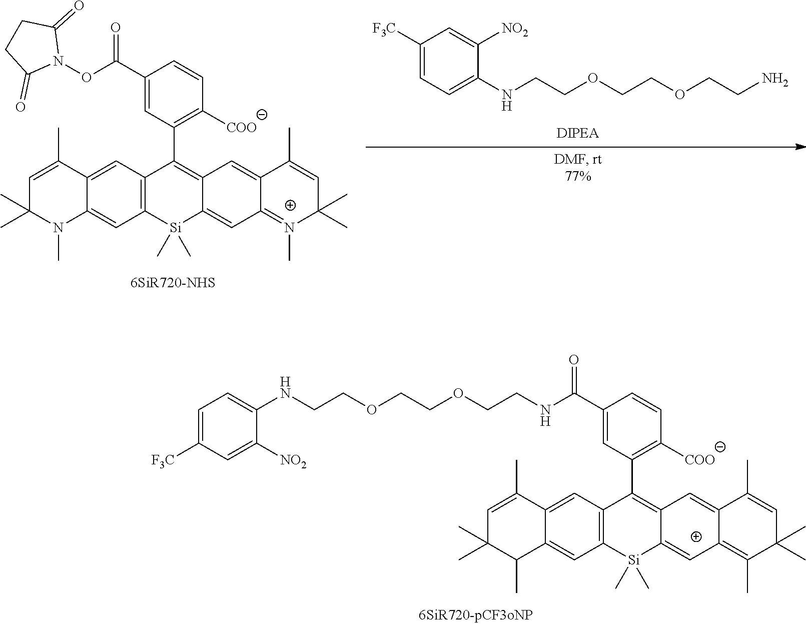

By the same scheme used in the synthesis of 6JF646-DNP, 6SiR720-pCF3 (7.1 mg, 0.0077 mmol) was acquired as a green solid (yield: 77%) using 6SiR720-NHS and pCF3-amine as starting materials.

¹H NMR (400 MHz, (CD₃)₂CO): δ 8.49 (br s, 1H), 8.36-6.35 (m, 1H), 8.10 (dd, 1H, J=8.0, 1.6 Hz), 8.01 (dd, 1H, J=8.0, 0.4 Hz), 7.97 (br t, 1H, J=5.6 Hz), 7.81 (dd, 1H, J=1.2, 0.8 Hz), 7.72 (dd, 1H, J=9.2, 2.0 Hz), 7.18 (d, 1H, J=9.2 Hz), 6.89 (s, 2H), 6.56 (s, 2H), 5.35-5.34 (m, 2H), 3.74 (t, 2H, J=5.6 Hz), 3.63-3.59 (m, 6H), 3.55-3.51 (m, 4H), 2.94 (s, 6H), 1.614 (s, 3H), 1.611 (s, 3H), 1.31 (s, 6H), 1.30 (s, 6H), 0.67 (s, 3H), 0.54 (s, 3H). HRMS (ESI⁺) calcd. for [M+H]⁺, 924.39739; found, 924.39648 (Δ−0.91mmu).

(3) Calculation of Dissociation Rate Constant of Anti-DNP scFv and Probe

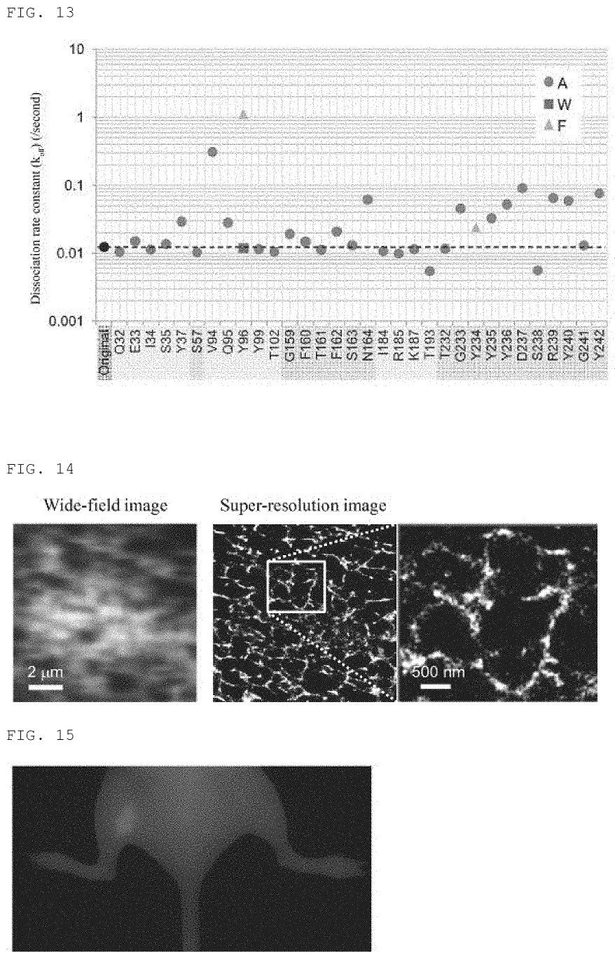

A flow channel in a stopped-flow apparatus (Bio-logic) was filled with a pretreatment liquid in which 1% w/v gelatin was dissolved in a phosphate buffer solution (pH 7.4) (PBS), and blocking was performed by leaving the apparatus at room temperature for at least 30 minutes, after which the flow channel was thoroughly washed with Milli-Q water. Using the apparatus, PBS in which MBP-5D4 or a variant thereof at a concentration of 100 nM or less and a probe at a concentration of 1 μM were dissolved, and PBS in which 100 μM DNP as a competitive substance was dissolved were mixed at a 1:1 ratio, and a fluorescence change was measured. At this time, the excitation/fluorescence wavelengths used were 510 nm/529-556 nm for the DCF probe and 650 nm/672-712 nm for the SiR probe, and measurement was performed under conditions of a temperature of 25-27C. A dissociation rate constant koff was calculated by fitting the fluorescence intensity I(t) observed with respect to time t using formula (1) below (FIG. 13 and Table 1).

I(t)=c₁·e^{-k}^{_{off}}^{t}+c₂·t+c₃ Formula (1):

In the formula cₙ (n=1-3) is a constant.

Alanine-scanning mutagenesis was performed on an scFv variable region, and the dissociation rate of each mutant and 6DCF-DNP was calculated from competition experiments with DNP, and a V94A mutant was found to have a dissociation rate of 0.31 s⁻¹, which was a rate increase of 25 times the original rate. Focusing on the 96^{th} and 234^{th} tyrosines, alanine mutation of which produces dead mutants in which no fluorescence change in competition experiments is observed, when mutants in which the 96^{th} and 234^{th} tyrosines were replaced with another aromatic amino acid were evaluated, it was clear that the dissociation rate of the Y96F mutant was made 100 times faster, being 1.1 s⁻¹ (see FIG. 13).

Furthermore, the results of evaluating the dissociation rate with 5D4 or 5D4 (Y96F) for the 6SiR-DNP derivatives obtained in synthesis examples 16 through 35 are shown in Table 1. As indicated in Table 1, the 5D4 (Y96F) 6SiR-pCNoNP combination had the highest dissociation rate of 14 s⁻¹.

TABLE 1
Probe dissociation characteristics

| Probe | R [symbol] | [symbol] | Tag | [symbol] |
|---|---|---|---|---|
| 6SiR-DNP | NO [symbol] | p-NO [symbol] | Original | 0.021 |
| 6SiR-pBroNP | NO [symbol] | p-Br | Original | 0.045 |
| 6SiR-pSO2MeoNO | NO [symbol] | p-SO [symbol] Me | Original | 0.046 |
| 6SiR-pCl [symbol] NP | NO [symbol] | p-Cl | Original | 0.048 |
| 6SiR-mCNoNP | NO [symbol] | m-CN | Original | 0.091 |
| 6SiR-pCNoNP | NO [symbol] | p-CN | Original | 0.15 |
| 6SiR-pCOOMeoNP | NO [symbol] | p-COOMe | Original | 0.31 |
| 6SiR-DCF3P | CF [symbol] | p-CF [symbol] | Original | 0.37 |
| 6SiR-pCONHM [symbol] NP | NO [symbol] | p-CONHMe | Original | 0.84 |
| 6SiR-mCOOM [symbol] NP | NO [symbol] | m-COOMe | Original | 3.3 |
| 6SiR- [symbol] NP | NO [symbol] | H | Original | 5. [symbol] |
| 6SiR-pNP | H | p-NO [symbol] | Original | N.D. |
| 6SiR-pMeoNP | NO [symbol] | p-Me | Original | N.D. |
| 6SiR-pMeOoNP | NO [symbol] | p-MeO | Original | N.D. |
| 6SiR-mCF3 [symbol] NP | NO [symbol] | m-CF [symbol] | Original | 0.0084 |
| 6SiR-pCF3oNP | NO [symbol] | p-CF [symbol] | Original | 0.0015 |
| 6SiR-pCF3OoNP | NO [symbol] | p-OCF [symbol] | Original | 0.0014 |
| 6SiR-DNP | NO [symbol] | p-NO [symbol] | Y96F | 2.3 |
| 6SiR-pCN [symbol] NP | NO [symbol] | p-CN | Y96F | 14 |

[symbol]

indicates data missing or illegible when filed

(4) Preparation of 5D4 (Y96F) ER Expression Construct

A 5D4 (Y96F) ER expression construct was introduced by circular PCR using two types of primers (Y96F_F and VLCDR3) with pECFP-5D4 as the template. The resultant linear PCR product was circularized using a Ligation Kit Version 2 (TAKARA), and an MBP-5D4 (Y96F) expression construct pECFP-5D4 (Y96F) was obtained.

(5) Super-Resolution Imaging by Single-Molecule Localization

An ECFP-5D4 (Y96F) expression plasmid (pECFP-5D4 (Y96F)-ER) to which an endoplasmic reticulum localization signal sequence was added was introduced to HeLa cells cultured on a 96-well plate, using Lipofectamine 2000. At 20 hours after plasmid introduction, the cells were treated with trypsin/EDTA and stripped, and then re-seeded on a cover glass coated with collagen/poly-L-lysine. At 20 hours after re-seeding, the cells were washed with HBS, loaded with 10 nM 6SiR-DNP, and subjected to super-resolution imaging. A specimen was excited using a 640 nm semiconductor laser, and fluorescence intermittency images of a single molecule were continuously acquired at an exposure of 16 milliseconds by a backside-illuminated cooled EM-CCD camera (iXon, Andor Technology). A centroid position of a bright point of single-molecule fluorescence in each image was determined, and a super-resolution image was reconstructed. In the super-resolution image, the structure of the endoplasmic reticulum was visualized with high spatial resolution relative to a normal fluorescence microscope image (FIG. 14).

### Example 6

In Vivo Imaging

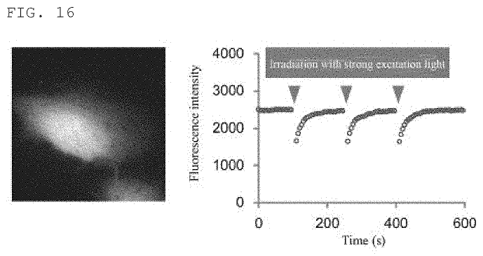

Two million SKOV3 cells stably expressing EGFP-5D4 or EGFP were subcutaneously injected at the base of each of the left and right thighs of a seven-week-old female nude mouse BALB/c-nu/nu (Japan SLC) reared for five days on an autofluorescence reduction chow D10001 (RESEARCH DIETS Inc.). After being reared for five more days using the autofluorescence reduction chow, the mouse was subjected to an in vivo imaging experiment. The mouse was anaesthetized with isoflurane, after which 10 μM 6SiR700-pCF3oNP dissolved in 100 μL of PBS was administered intravenously, and observation was performed using a Pearl Trilogy (LI-COR, Inc.) fluorescence imager. The excitation/fluorescence wavelengths used were 685 nm/720 nm. Five minutes after administration of the probe, SKOV3 cells expressing EGFP-5D4 were specifically visualized (FIG. 15). This result clearly indicates that in vivo labeling of a target cell using near-infrared fluorescence is possible by the tag/probe method developed herein.

### Example 7

Long-Term-Stable Fluorescence Imaging Based on Tag/Probe Binding/Dissociation Equilibrium

HeLa cells expressing ECFP-5D4 were immersed for one hour at room temperature in HBS including 10 nM 6SiR-pCOONHMeoNP, and fluorescence imaging thereof was performed without modification of this state with the probe present at a concentration of 10 nM in the extracellular fluid. When a whole cell was irradiated with intense excitation light, recovery of the fluorescence signal after photobleaching was observed (FIG. 16). This phenomenon results in a constant fluorescence intensity being observed on the basis of the equilibrium of binding and dissociation of the tag and the probe, and this result indicates that stable fluorescence observation is possible. In super-resolution imaging and other fluorescence observation in which photobleaching has hitherto been a serious problem, the tag/probe method developed herein has the revolutionary characteristic of making it possible to obtain a semi-permanent fluorescence signal without limitation by photobleaching.

### SEQUENCE LISTING

1. A method for fluorescently labeling an intracellular protein, said method comprising:
obtaining, in a cell, a fusion protein of a labeling object protein and an anti-DNP (dinitrophenyl compound) antibody;
bringing a compound represented by formula (I) or a salt thereof into contact with said cell; and
fluorescently labeling said object protein by reacting said fusion protein and the compound represented by formula (I) or a salt thereof. 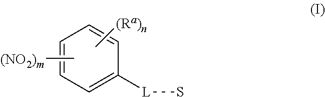
(In said formula (I):
S is a fluorescent group,
L is a linker, and
R^{a} is a monovalent substituent;
m is an integer of 0 to 2, and
n is an integer of 0 to 2;
when m is 2, n is 0;
when m is 1, n is 1 or 0;
when m is 0, n is 2; and
when n is 2, the monovalent substituents of R^{a} may be the same or different.)

2. The method according to claim 1, wherein the monovalent substituent represented by R^{a} is selected from the group consisting of a halogen atom, a C1-10 alkyl group, a C1-10 alkoxy group, a cyano group, an ester group, an amide group, an alkyl sulfonyl group, a C1-10 alkyl group in which at least one hydrogen atom is substituted with a fluorine atom, and a C1-10 alkoxy group in which at least one hydrogen atom is substituted with a fluorine atom.

3. A method for fluorescently labeling an intracellular protein, said method comprising:
obtaining, in a cell, a fusion protein of a labeling object protein and an anti-DNP (dinitrophenyl compound) antibody;
bringing a compound represented by formula (Ia) or a salt thereof into contact with said cell, and
fluorescently labeling said object protein by reacting said fusion protein and the compound represented by formula (Ia) or a salt thereof. 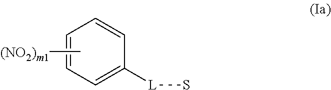
(In formula (1a):
S is a fluorescent group,
L is a linker, and
m1 is 1 or 2.)

4. The method according to claim 1, wherein:
said anti-DNP antibody in said fusion protein is an anti-DNP antibody or an antigen-binding fragment thereof comprising
a light chain including a VL-CDR1 comprising the amino acid sequence represented by SEQ ID NO: 1, a VL-CDR2 comprising the amino acid sequence represented by SEQ ID NO: 2, and a VL-CDR3 comprising the amino acid sequence represented by SEQ ID NO: 3, and
a heavy chain including a VH-CDR1 comprising the amino acid sequence represented by SEQ ID NO: 4, a VH-CDR2 comprising the amino acid sequence represented by SEQ ID NO: 5, and a VH-CDR3 comprising the amino acid sequence represented by SEQ ID NO: 6.
Sequence No. 1:
QEISGY
Sequence No. 2:
AAS
Sequence No. 3:
VQYASYPYT
Sequence No. 4:
GFTFSNYWMNW
Sequence No. 5:
IRLKSNNYAT
Sequence No. 6:
TGYYYDSRYGY

5. The method according to claim 4, wherein said anti-DNP antibody or antigen-binding fragment thereof is a single-chain Fv (scFv).

6. The method according to claim 4, wherein said anti-DNP antibody comprises an amino acid sequence having at least 90% homology to the amino acids of SEQ ID NO: 7, and includes amino acid sequences represented by SEQ ID NO: 1 through 6.
Sequence No. 7:
MADYKDIVLTQSPSSLSASLGERVSLTCRSSQEISGYLGWLQQKPDGSIK
RLIYAASTLDSGVPKRFSGSRSGSDYSLTISSLESEDFADYYCVQYASYP
YTFGGGTKLEMKRGGGGSGGGGSGGGGSGGGGSQIQLQESGGGLVQPGGS
MKLSCVASGFTFSNYWMNWVRQSPEKGLDWVAEIRLKSNNYATHYAESVK
GRFTISRDDSKSSVYLQMNNLRAEDTGIYYCTGYYYDSRYGYWGQGTTVT
VSS

7. The method according to claim 6, wherein said amino acid sequence is SEQ ID NO: 7.

8. The method according to claim 1, wherein said anti-DNP antibody in said fusion protein comprises an amino acid sequence having at least 90% homology to the amino acids of SEQ ID NO: 7 and includes the amino acid sequences represented by SEQ ID NO: 1 to 6,
and comprises an amino acid sequence in which at least one of substitutions below is made in the amino acid sequence represented by any of SEQ ID NO: 1 to 6:
(a) any one amino acid from among glutamic acid at position 33, tyrosine at position 37, valine at position 94, glutamine at position 95, glycine at position 159, phenylalanine at position 160, phenylalanine at position 162, asparagine at position 164, glycine at position 233, tyrosine at position 235, tyrosine at position 236, aspartic acid at position 237, arginine at position 239, tyrosine at position 240, and tyrosine at position 242 numbered from an N-terminus is substituted with alanine; or
(b) any one amino acid from among tyrosine at position 96 and tyrosine at position 234 numbered from the N-terminus is substituted with phenylalanine.

9. The method according to claim 1, wherein said anti-DNP antibody in said fusion protein comprises an amino acid sequence in which a substitution below is made in the amino acids of SEQ ID NO: 7:
(1) any one amino acid from among glutamic acid at position 33, tyrosine at position 37, valine at position 94, glutamine at position 95, glycine at position 159, phenylalanine at position 160, phenylalanine at position 162, asparagine at position 164, glycine at position 233, tyrosine at position 235, tyrosine at position 236, aspartic acid at position 237, arginine at position 239, tyrosine at position 240, and tyrosine at position 242 numbered from the N-terminus is substituted with alanine; or
(2) any one amino acid from among tyrosine at position 96 and tyrosine at position 234 numbered from the N-terminus is substituted with phenylalanine.

10. The method according to claim 1, wherein obtaining said fusion protein includes obtaining a polynucleotide coding for said fusion protein, obtaining a plasmid or vector capable of expressing said fusion protein, causing said fusion protein to be expressed in a cell, or isolating said expressed fusion protein.

11. The method according to claim 1, wherein said linker is represented by T-Y, where Y represents a bonding group for bonding with the fluorescent group S, and T represents a crosslinking group.

12. The method according to claim 11, wherein said bonding group is selected from an amide group, an alkylamide group, carbonylamino group, an ester group, an alkylester group, or an alkylether group.

13. The method according to claim 1, wherein S is represented by formula (II) below. 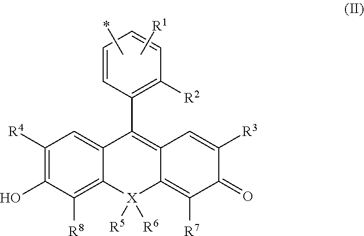
(In formula (II): R¹ represents a hydrogen atom or one to four same or different monovalent substituents which are present on a benzene ring;
R² represents a hydrogen atom, a monovalent substituent, or a bond;
R³ and R⁴ each independently represent a hydrogen atom, a C1-6 alkyl group, or a halogen atom;
R⁵ and R⁶ each independently represent a C1-6 alkyl group, an aryl group, or a bond, provided that R⁵ and R⁶ being absent when X is an oxygen atom;
R⁷ and R⁸ each independently represent a hydrogen atom, a C1-6 alkyl group, a halogen atom, or a bond;
X represents an oxygen atom or a silicon atom; and
* represents a location of bonding with L in formula (I) at any position on the benzene ring.)

14. The method according to claim 1, wherein S is represented by formula (III) below. 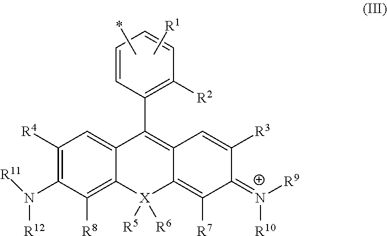
(In formula (III): R¹ to R⁸ and X are as defined in formula (II);
R⁹ and R¹⁰ each independently represent a hydrogen atom or a C1-6 alkyl group;
R⁹ and R¹⁰ may also together form a 4- to 7-membered heterocyclyl which includes a nitrogen atom to which R⁹ and R¹⁰ are bonded;
either R⁹ or R¹⁰, or both R⁹ and R¹⁰ may also respectively combine with R³ or R⁷ to form a 5- to 7-membered heterocyclyl or heteroaryl which includes a nitrogen atom to which R⁹ or R¹⁰ is bonded, and may comprise one to three additional hetero atoms selected from the group consisting of an oxygen atom, a nitrogen atom, and a sulfur atom as ring-forming members, and the heterocyclyl or heteroaryl may be furthermore substituted with a C1-6 alkyl, a C2-6 alkenyl, or a C2-6 alkynyl, a C6-10 aralkyl group, or a C6-10 alkyl-substituted alkenyl group;
R¹¹ and R¹² each independently represent a hydrogen atom or a C1-3 alkyl group;
R¹¹ and R¹² may also together form a 4- to 7-membered heterocyclyl which includes a nitrogen atom to which R¹¹ and R¹²are bonded;
either R¹¹ or R¹², or both R¹¹ and R¹² may also respectively combine with R⁴ or R⁸ to form a 5- to 7-membered heterocyclyl or heteroaryl which includes a nitrogen atom to which R¹¹ or R¹² is bonded, and may comprise one to three additional hetero atoms selected from the group consisting of an oxygen atom, a nitrogen atom, and a sulfur atom as ring-forming members, and the heterocyclyl or heteroaryl may be furthermore substituted with a C1-6 alkyl, a C2-6 alkenyl, or a C2-6 alkynyl, a C6-10 aralkyl group, or a C6-10 alkyl-substituted alkenyl group; and
* represents a location of bonding with L in formula (I) at any position on the benzene ring.)

15. An anti-DNP antibody or an antigen-binding fragment thereof comprising:
a light chain including a VL-CDR1 comprising the amino acid sequence represented by SEQ ID NO: 1, a VL-CDR2 comprising the amino acid sequence represented by SEQ ID NO: 2, and a VL-CDR3 comprising the amino acid sequence represented by SEQ ID NO: 3; and
a heavy chain including a VH-CDR1 comprising the amino acid sequence represented by SEQ ID NO: 4, a VH-CDR2 comprising the amino acid sequence represented by SEQ ID NO: 5, and a VH-CDR3 comprising the amino acid sequence represented by SEQ ID NO: 6.
Sequence No. 1:
QEISGY
Sequence No. 2:
AAS
Sequence No. 3:
VQYASYPYT
Sequence No. 4:
GFTFSNYWMNW
Sequence No. 5:
IRLKSNNYAT
Sequence No. 6:
TGYYYDSRYGY

16. The anti-DNP antibody or antigen-binding fragment thereof according to claim 15, wherein said anti-DNP antibody or antigen-binding fragment thereof is a single-chain Fv (scFv).

17. The anti-DNP antibody or antigen-binding fragment thereof according to claim 15, comprising an amino acid sequence having at least 90% homology to SEQ ID NO: 7 and including amino acid sequences represented by SEQ ID NO: 1 to 6.
Sequence No. 7:
MADYKDIVLTQSPSSLSASLGERVSLTCRSSQEISGYLGWLQQKPDGSIK
RLIYAASTLDSGVPKRFSGSRSGSDYSLTISSLESEDFADYYCVQYASYP
YTFGGGTKLEMKRGGGGSGGGGSGGGGSGGGGSQIQLQESGGGLVQPGGS
MKLSCVASGFTFSNYWMNWVRQSPEKGLDWVAEIRLKSNNYATHYAESVK
GRFTISRDDSKSSVYLQMNNLRAEDTGIYYCTGYYYDSRYGYWGQGTTVT
VSS

18. The anti-DNP antibody or antigen-binding fragment thereof according to claim 17, wherein said amino acid sequence is SEQ ID NO: 7.

19. An anti-DNP antibody or an antigen-binding fragment thereof, comprising an amino acid sequence having at least 90% homology to the amino acids of SEQ ID NO: 7 and including the amino acid sequences represented by SEQ ID NO: 1 to 6, and comprising an amino acid sequence in which at least one of substitutions below is made in the amino acid sequence represented by any of SEQ ID NO: 1 to 6:
(a) any one amino acid from among glutamic acid at position 33, tyrosine at position 37, valine at position 94, glutamine at position 95, glycine at position 159, phenylalanine at position 160, phenylalanine at position 162, asparagine at position 164, glycine at position 233, tyrosine at position 235, tyrosine at position 236, aspartic acid at position 237, arginine at position 239, tyrosine at position 240, and tyrosine at position 242 numbered from the N-terminus is substituted with alanine; or
(b) any one amino acid from among tyrosine at position 96 and tyrosine at position 234 numbered from the N-terminus is substituted with phenylalanine.

20. An anti-DNP antibody or an antigen-binding fragment thereof, comprising an amino acid sequence in which a substitution below is made in the amino acids of SEQ ID NO: 7:
(1) any one amino acid from among glutamic acid at position 33, tyrosine at position 37, valine at position 94, glutamine at position 95, glycine at position 159, phenylalanine at position 160, phenylalanine at position 162, asparagine at position 164, glycine at position 233, tyrosine at position 235, tyrosine at position 236, aspartic acid at position 237, arginine at position 239, tyrosine at position 240, and tyrosine at position 242 numbered from the N-terminus is substituted with alanine; or
(2) any one amino acid from among tyrosine at position 96 and tyrosine at position 234 numbered from the N-terminus is substituted with phenylalanine.

21. An isolated nucleic acid coding for the antibody or antigen-binding fragment thereof according to claim 15.

22. The nucleic acid according to claim 21, comprising a base sequence represented by SEQ ID NO: 8.
Sequence No. 8:
ATGGCGGACTACAAAGACATTGTGCTGACCCAGTCTCCATCCTCTTTATC
TGCCTCTCTGGGAGAAAGAGTCAGTCTCACTTGTCGGTCAAGTCAGGAAA
TTAGTGGTTACTTAGGCTGGCTTCAGCAGAAACCAGATGGAAGTATTAAA
CGCCTGATCTACGCCGCATCCACTTTAGATTCTGGTGTCCCAAAAAGGTT
CAGTGGCAGTAGGTCTGGGTCAGATTATTCTCTCACCATCAGCAGCCTTG
AGTCTGAAGATTTTGCAGACTATTATTGTGTACAATATGCTAGTTATCCG
TACACGTTCGGAGGGGGGACCAAGCTGGAAATGAAACGCGGTGGTGGTGG
TTCTGGTGGTGGTGGTTCTGGCGGCGGCGGCTCCGGTGGTGGTGGATCCC
AGATTCAGCTTCAGGAGTCTGGAGGAGGCTTGGTGCAACCTGGAGGATCC
ATGAAACTCTCCTGTGTTGCCTCTGGATTCACTTTCAGTAACTACTGGAT
GAACTGGGTCCGCCAGTCTCCAGAGAAGGGGCTTGACTGGGTTGCTGAAA
TTAGATTGAAATCTAATAATTATGCAACACATTATGCGGAGTCTGTGAAA
GGGAGGTTCACCATCTCAAGAGATGATTCCAAAAGTAGTGTCTACCTGCA
AATGAACAACTTAAGAGCTGAAGACACTGGCATTTATTACTGTACCGGTT
ATTACTACGATAGTAGGTACGGCTACTGGGGCCAAGGCACCACGGTCACC
GTCTCCTCGGCCTCG

23. An isolated nucleic acid coding for the antibody or antigen-binding fragment according to claim 20.

24. A plasmid or vector including the nucleic acid according to claim 21.

25. A fluorescent probe used in the method according to claim 1, comprising said compound represented by formula (I) or a salt thereof. 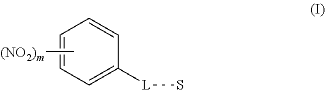
(In said formula (I):
S is a fluorescent group,
L is a linker, and
m is an integer of 1 or 2.)

26. The fluorescent probe according to claim 25, used for in vivo imaging.

27. A compound represented by a formula below, or a salt thereof.

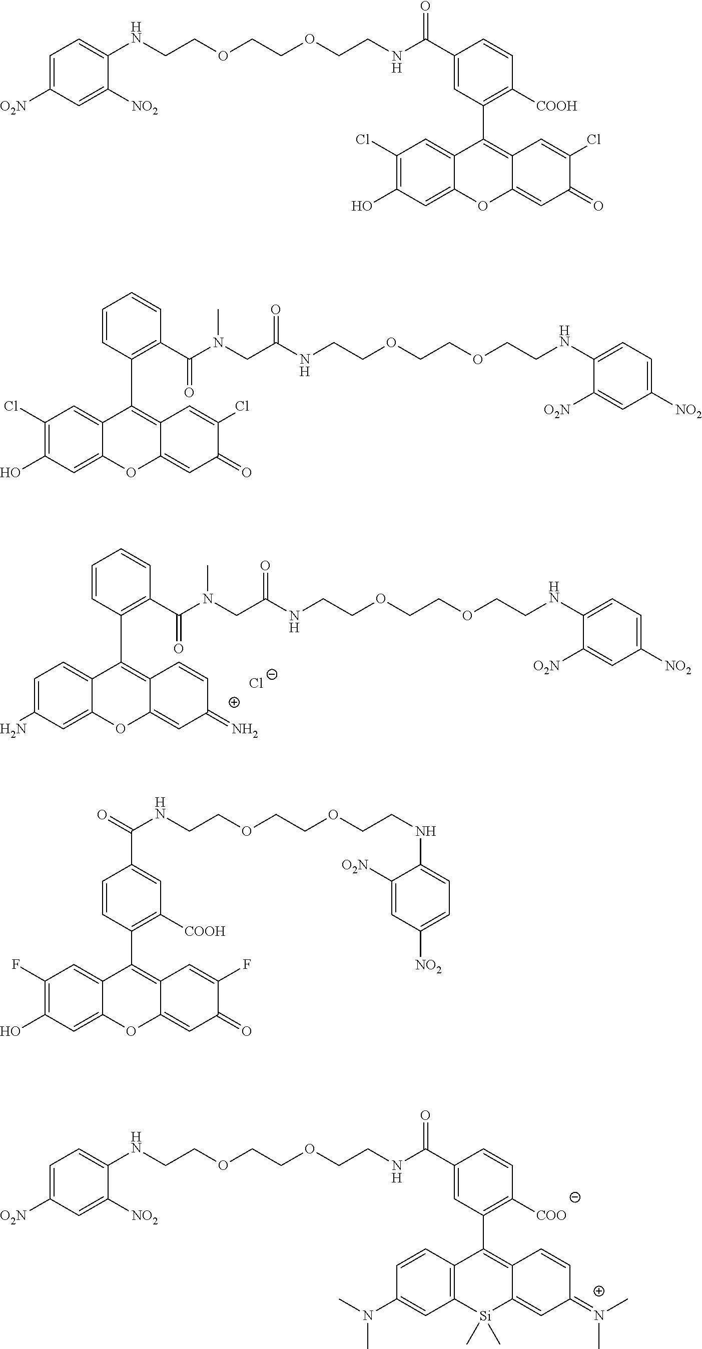

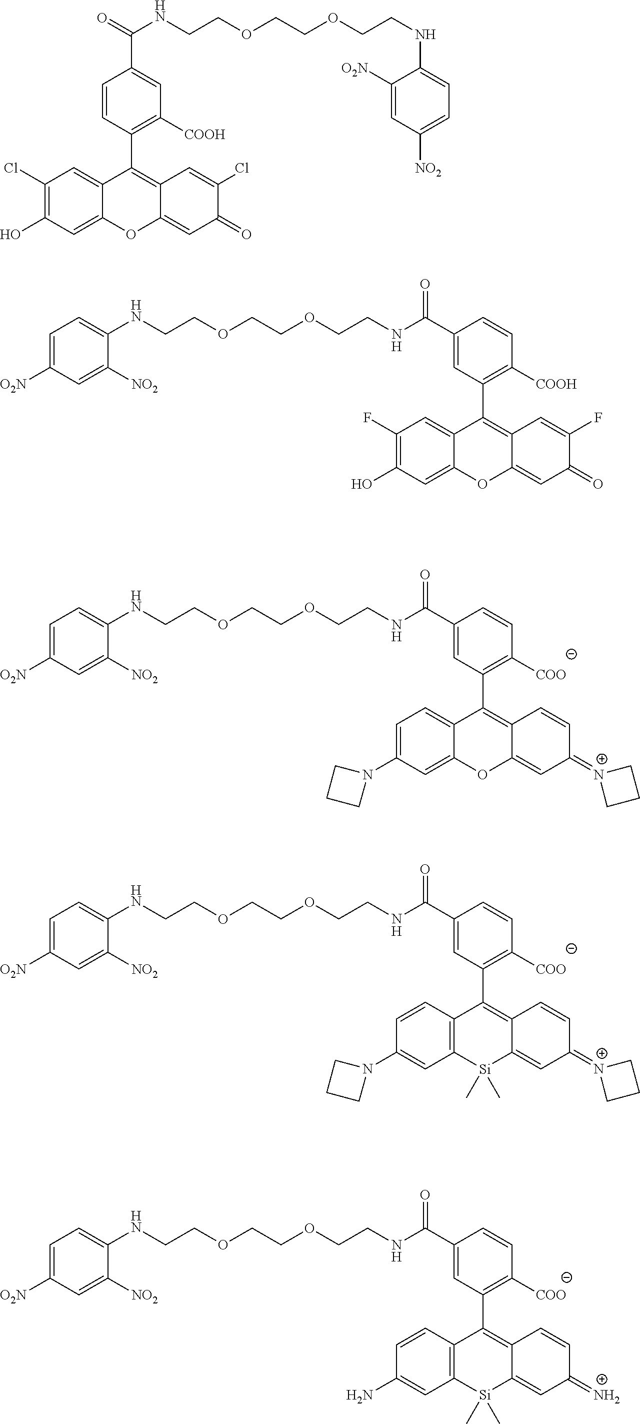

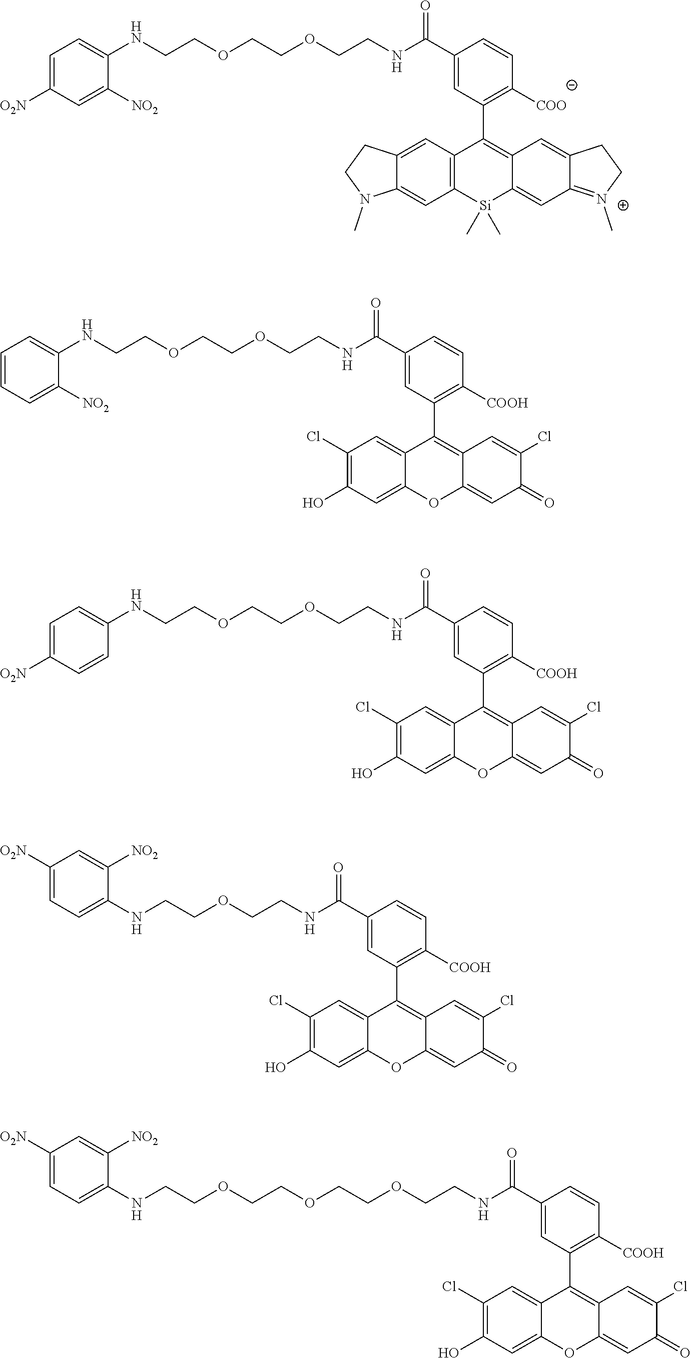

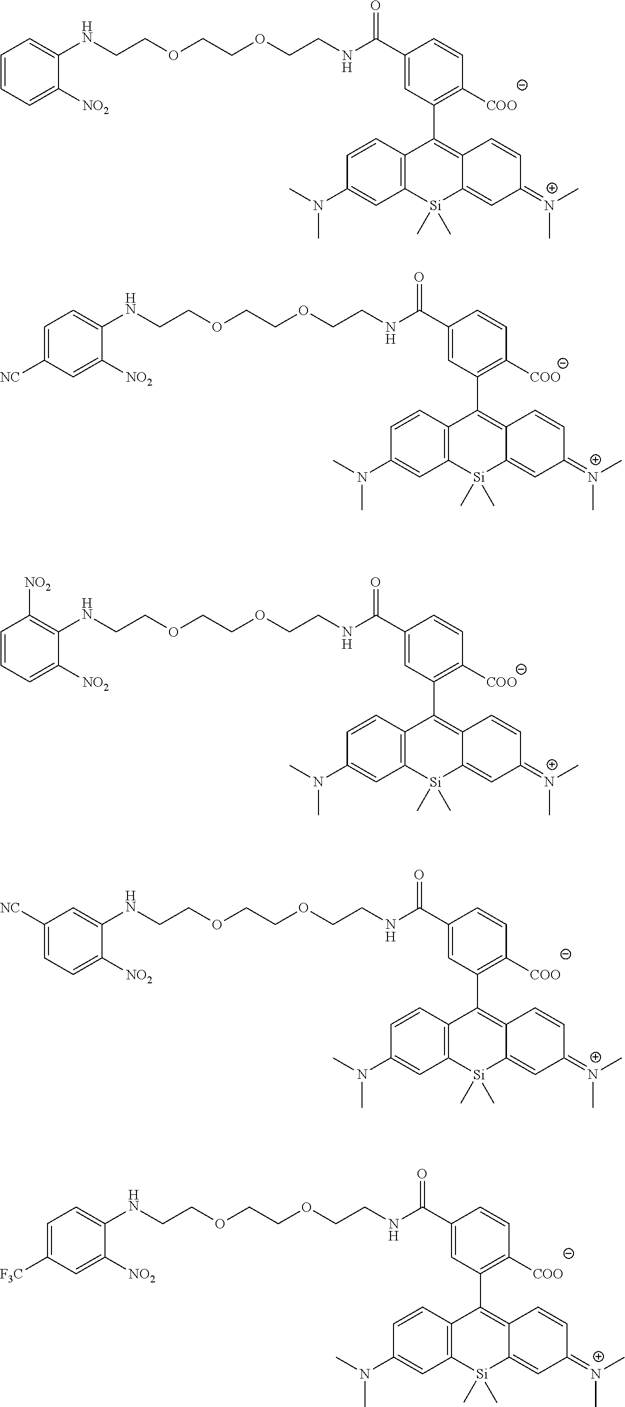

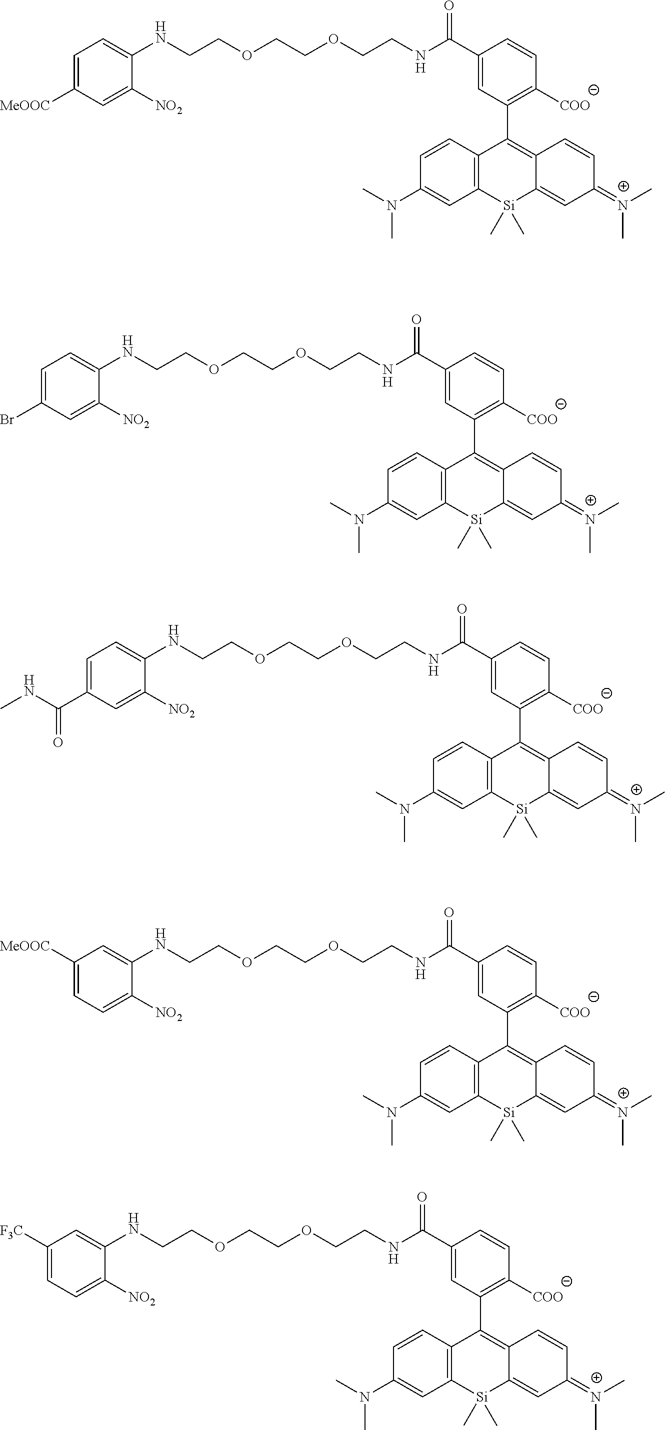

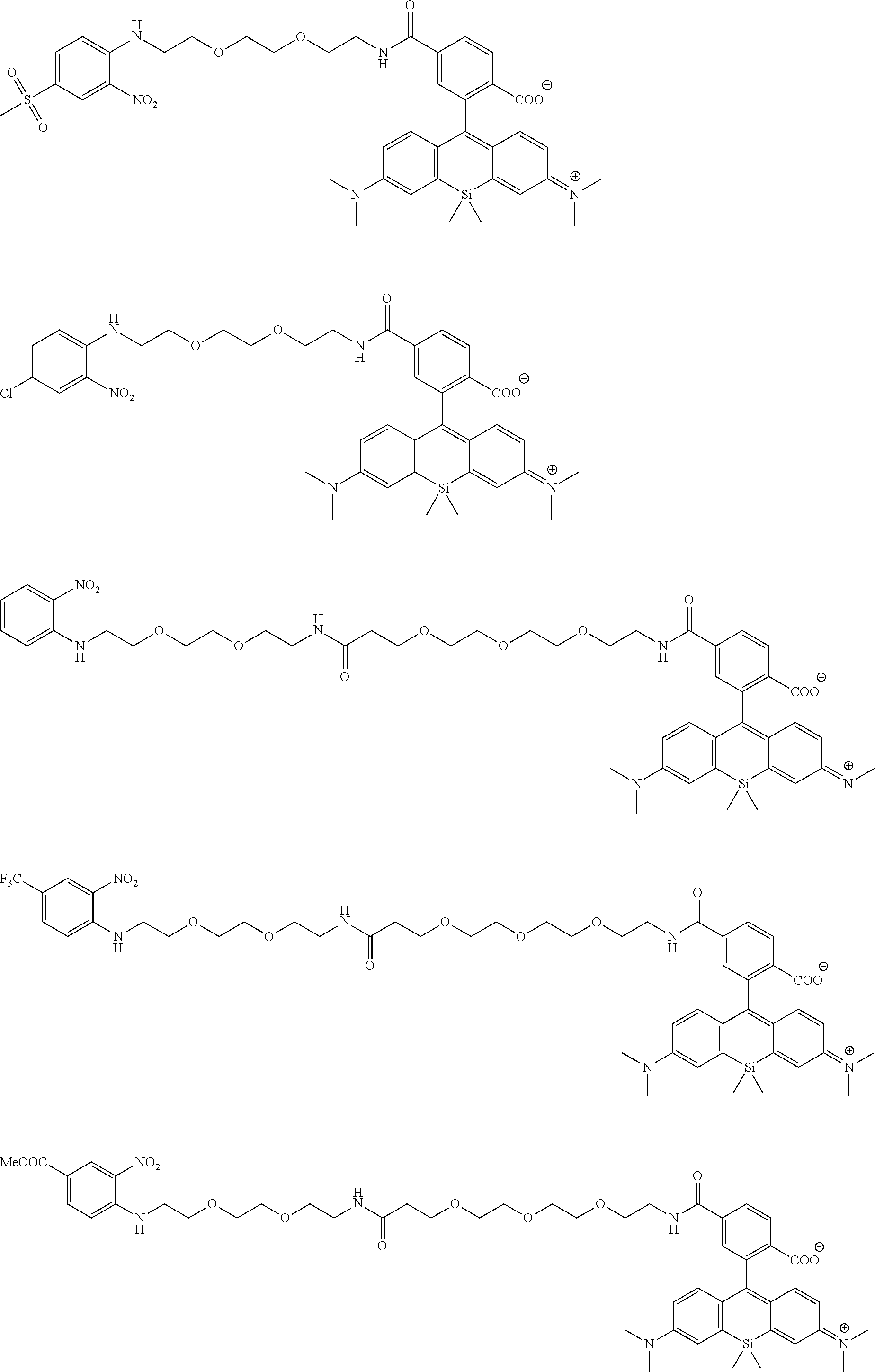

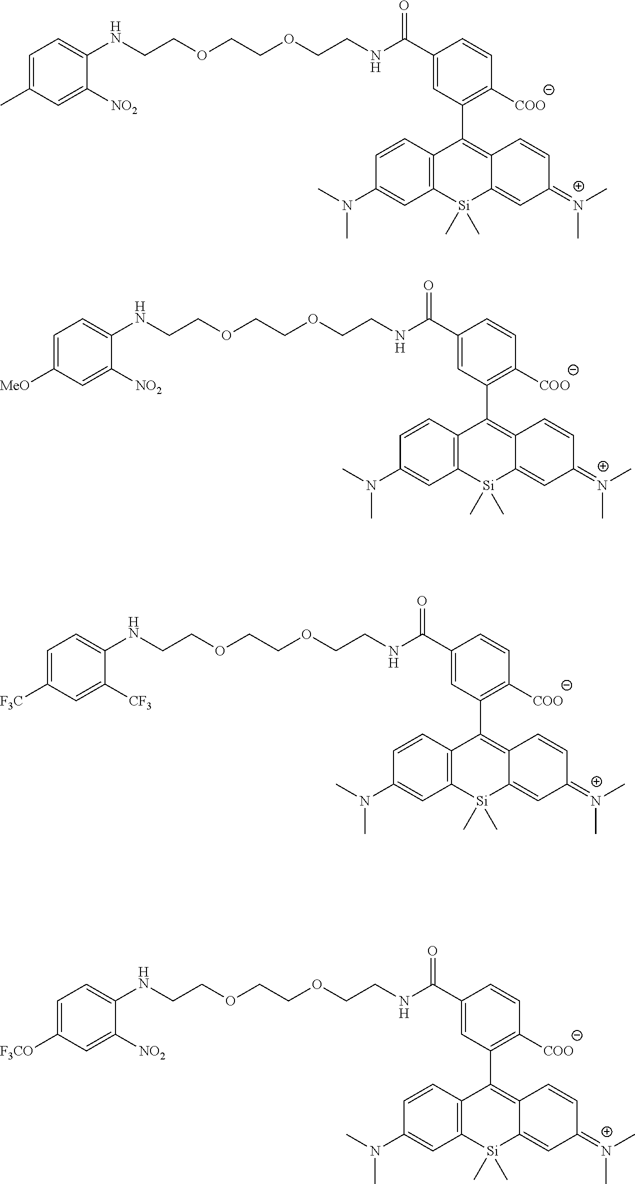

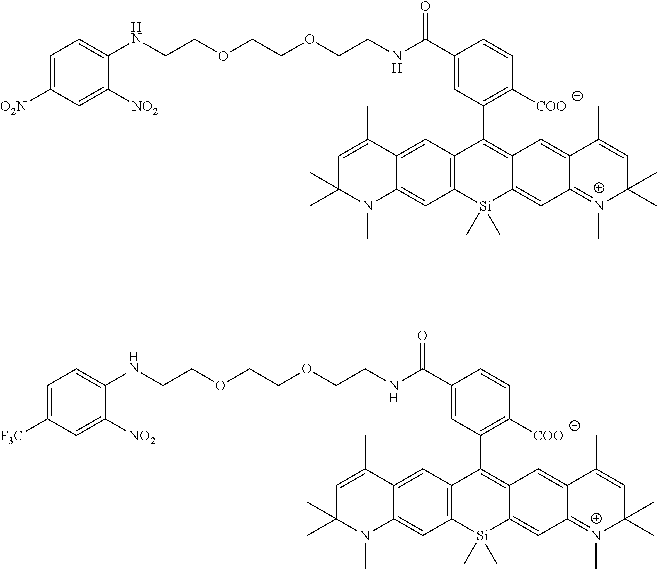

28. A super-resolution imaging method comprising:
obtaining, in a cell, a fusion protein of a labeling object protein and an anti-DNP (dinitrophenyl compound) antibody;
bringing a compound represented by formula (I) below or a salt thereof into contact with the cell; and
fluorescently labeling the object protein by reacting the fusion protein and the compound represented by formula (I) below or a salt thereof. 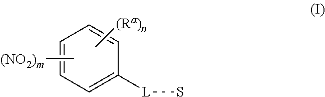
(In said formula (I):
S is a fluorescent group,
L is a linker, and
R^{a} is a monovalent substituent;
m is an integer of 0 to 2, and
n is an integer of 0 to 2;
when m is 2, n is 0;
when m is 1, n is 1 or 0;
when m is 0, n is 2; and
when n is 2, the monovalent substituents of R^{a} may be the same or different.)

29. The super-resolution imaging method according to claim 28, using single-molecule localization microscopy.

30. The super-resolution imaging method according to claim 28, wherein said anti-DNP antibody in said fusion protein comprises an amino acid sequence having at least 90% homology to the amino acids of SEQ ID NO: 7 and includes the amino acid sequences represented by SEQ ID NO: 1 to 6, and comprises an amino acid sequence in which at least one of substitutions below is made in the amino acid sequence represented by any of SEQ ID NO: 1 to 6:
(a) any one amino acid from among glutamic acid at position 33, tyrosine at position 37, valine at position 94, glutamine at position 95, glycine at position 159, phenylalanine at position 160, phenylalanine at position 162, asparagine at position 164, glycine at position 233, tyrosine at position 235, tyrosine at position 236, aspartic acid at position 237, arginine at position 239, tyrosine at position 240, and tyrosine at position 242 numbered from an N-terminus is substituted with alanine; or
(b) any one amino acid from among tyrosine at position 96 and tyrosine at position 234 numbered from the N-terminus is substituted with phenylalanine.

31. The super-resolution imaging method according to claim 28, wherein said anti-DNP antibody in said fusion protein comprises an amino acid sequence in which a substitution below is made in the amino acids of SEQ ID NO: 7:
(1) any one amino acid from among glutamic acid at position 33, tyrosine at position 37, valine at position 94, glutamine at position 95, glycine at position 159, phenylalanine at position 160, phenylalanine at position 162, asparagine at position 164, glycine at position 233, tyrosine at position 235, tyrosine at position 236, aspartic acid at position 237, arginine at position 239, tyrosine at position 240, and tyrosine at position 242 numbered from the N-terminus is substituted with alanine; or
(2) any one amino acid from among tyrosine at position 96 and tyrosine at position 234 numbered from the N-terminus is substituted with phenylalanine.

32. A fluorescent probe used in the super-resolution imaging method according to claim 28, said fluorescent probe comprising a compound represented by formula (I) below or a salt thereof. 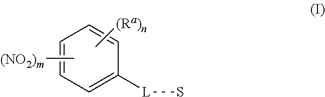
(In said formula (I): S is a fluorescent group, L is a linker, and R^{a} is a monovalent substituent; m is an integer of 0 to 2, n is an integer of 0 to 2; when m is 2, n is 0; when m is 1, n is 1 or 0; when m is 0, n is 2; and when n is 2, the monovalent substituents of R^{a} may be the same or different.)

33. The fluorescent probe according to claim 31, wherein the monovalent substituent represented by R^{a} is selected from the group consisting of a halogen atom, a C1-10 alkyl group, a C1-10 alkoxy group, a cyano group, an ester group, an amide group, an alkyl sulfonyl group, a C1-10 alkyl group in which at least one hydrogen atom is substituted with a fluorine atom, and a C1-10 alkoxy group in which at least one hydrogen atom is substituted with a fluorine atom.

34. The fluorescent probe used in a super-resolution imaging method according to claim 32, including a compound represented by formula (Ib) below or a salt thereof. 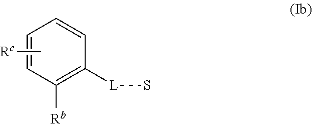
In formula (Ib), S is a fluorescent group, L is a linker, and R^{b} and R^{c} are selected from combinations below.
(R^{b}, R^{c}):(NO₂, p-NO₂), (NO₂, p-Br), (NO₂, p-SO₂Me), (NO₂, p-Cl), (NO₂, m-CN), (NO₂, p-CN), (NO₂, p-COOMe), (CF₃, p-CF₃), (NO₂, p-CONHMe), (NO₂, m-COOMe), (NO₂, H)
(Here, p- and m- represent R^{c} being in a para position and a meta position on the benzene ring, respectively, with respect to L.)


SEQUENCE LISTING (25 sequences)

SEQ ID NO: 1 - protein, 6 aa, Mus musculus
QEISGY

SEQ ID NO: 2 - protein, 3 aa, Mus musculus
AAS

SEQ ID NO: 3 - protein, 9 aa, Mus musculus
VQYASYPYT

SEQ ID NO: 4 - protein, 11 aa, Mus musculus
GFTFSNYWMNW

SEQ ID NO: 5 - protein, 10 aa, Mus musculus
IRLKSNNYAT

SEQ ID NO: 6 - protein, 11 aa, Mus musculus
TGYYYDSRYGY

SEQ ID NO: 7 - protein, 253 aa, Artificial Sequence
  analogous
MADYKDIVLTQSPSSLSASLGERVSLTCRSSQEISGYLGWLQQKPDGSIKRLIYAASTLDSGVPKRFSGSRSGSDYSLTISSLESEDFADYYCVQYASYPYTFGGGTKLEMKRGGGGSGGGGSGGGGSGGGGSQIQLQESGGGLVQPGGSMKLSCVASGFTFSNYWMNWVRQSPEKGLDWVAEIRLKSNNYATHYAESVKGRFTISRDDSKSSVYLQMNNLRAEDTGIYYCTGYYYDSRYGYWGQGTTVTVSS

SEQ ID NO: 8 - DNA, 765 bp, Artificial Sequence
  analogous
atggcggactacaaagacattgtgctgacccagtctccatcctctttatctgcctctctgggagaaagagtcagtctcacttgtcggtcaagtcaggaaattagtggttacttaggctggcttcagcagaaaccagatggaagtattaaacgcctgatctacgccgcatccactttagattctggtgtcccaaaaaggttcagtggcagtaggtctgggtcagattattctctcaccatcagcagccttgagtctgaagattttgcagactattattgtgtacaatatgctagttatccgtacacgttcggaggggggaccaagctggaaatgaaacgcggtggtggtggttctggtggtggtggttctggcggcggcggctccggtggtggtggatcccagattcagcttcaggagtctggaggaggcttggtgcaacctggaggatccatgaaactctcctgtgttgcctctggattcactttcagtaactactggatgaactgggtccgccagtctccagagaaggggcttgactgggttgctgaaattagattgaaatctaataattatgcaacacattatgcggagtctgtgaaagggaggttcaccatctcaagagatgattccaaaagtagtgtctacctgcaaatgaacaacttaagagctgaagacactggcatttattactgtaccggttattactacgatagtaggtacggctactggggccaaggcaccacggtcaccgtctcctcggcctcg

SEQ ID NO: 9 - protein, 253 aa, Artificial Sequence
  analogous
MADYKDIVLTQSPSSLSASLGERVSLTCRSSQAISGYLGWLQQKPDGSIKRLIYAASTLDSGVPKRFSGSRSGSDYSLTISSLESEDFADYYCVQYASYPYTFGGGTKLEMKRGGGGSGGGGSGGGGSGGGGSQIQLQESGGGLVQPGGSMKLSCVASGFTFSNYWMNWVRQSPEKGLDWVAEIRLKSNNYATHYAESVKGRFTISRDDSKSSVYLQMNNLRAEDTGIYYCTGYYYDSRYGYWGQGTTVTVSS

SEQ ID NO: 10 - protein, 253 aa, Artificial Sequence
  analogous
MADYKDIVLTQSPSSLSASLGERVSLTCRSSQEISGALGWLQQKPDGSIKRLIYAASTLDSGVPKRFSGSRSGSDYSLTISSLESEDFADYYCVQYASYPYTFGGGTKLEMKRGGGGSGGGGSGGGGSGGGGSQIQLQESGGGLVQPGGSMKLSCVASGFTFSNYWMNWVRQSPEKGLDWVAEIRLKSNNYATHYAESVKGRFTISRDDSKSSVYLQMNNLRAEDTGIYYCTGYYYDSRYGYWGQGTTVTVSS

SEQ ID NO: 11 - protein, 253 aa, Artificial Sequence
  analogous
MADYKDIVLTQSPSSLSASLGERVSLTCRSSQEISGYLGWLQQKPDGSIKRLIYAASTLDSGVPKRFSGSRSGSDYSLTISSLESEDFADYYCAQYASYPYTFGGGTKLEMKRGGGGSGGGGSGGGGSGGGGSQIQLQESGGGLVQPGGSMKLSCVASGFTFSNYWMNWVRQSPEKGLDWVAEIRLKSNNYATHYAESVKGRFTISRDDSKSSVYLQMNNLRAEDTGIYYCTGYYYDSRYGYWGQGTTVTVSS

SEQ ID NO: 12 - protein, 253 aa, Artificial Sequence
  analogous
MADYKDIVLTQSPSSLSASLGERVSLTCRSSQEISGYLGWLQQKPDGSIKRLIYAASTLDSGVPKRFSGSRSGSDYSLTISSLESEDFADYYCVAYASYPYTFGGGTKLEMKRGGGGSGGGGSGGGGSGGGGSQIQLQESGGGLVQPGGSMKLSCVASGFTFSNYWMNWVRQSPEKGLDWVAEIRLKSNNYATHYAESVKGRFTISRDDSKSSVYLQMNNLRAEDTGIYYCTGYYYDSRYGYWGQGTTVTVSS

SEQ ID NO: 13 - protein, 253 aa, Artificial Sequence
  analogous
MADYKDIVLTQSPSSLSASLGERVSLTCRSSQEISGYLGWLQQKPDGSIKRLIYAASTLDSGVPKRFSGSRSGSDYSLTISSLESEDFADYYCVQFASYPYTFGGGTKLEMKRGGGGSGGGGSGGGGSGGGGSQIQLQESGGGLVQPGGSMKLSCVASGFTFSNYWMNWVRQSPEKGLDWVAEIRLKSNNYATHYAESVKGRFTISRDDSKSSVYLQMNNLRAEDTGIYYCTGYYYDSRYGYWGQGTTVTVSS

SEQ ID NO: 14 - protein, 253 aa, Artificial Sequence
  analogous
MADYKDIVLTQSPSSLSASLGERVSLTCRSSQEISGYLGWLQQKPDGSIKRLIYAASTLDSGVPKRFSGSRSGSDYSLTISSLESEDFADYYCVQYASYPYTFGGGTKLEMKRGGGGSGGGGSGGGGSGGGGSQIQLQESGGGLVQPGGSMKLSCVASAFTFSNYWMNWVRQSPEKGLDWVAEIRLKSNNYATHYAESVKGRFTISRDDSKSSVYLQMNNLRAEDTGIYYCTGYYYDSRYGYWGQGTTVTVSS

SEQ ID NO: 15 - protein, 253 aa, Artificial Sequence
  analogous
MADYKDIVLTQSPSSLSASLGERVSLTCRSSQEISGYLGWLQQKPDGSIKRLIYAASTLDSGVPKRFSGSRSGSDYSLTISSLESEDFADYYCVQYASYPYTFGGGTKLEMKRGGGGSGGGGSGGGGSGGGGSQIQLQESGGGLVQPGGSMKLSCVASGATFSNYWMNWVRQSPEKGLDWVAEIRLKSNNYATHYAESVKGRFTISRDDSKSSVYLQMNNLRAEDTGIYYCTGYYYDSRYGYWGQGTTVTVSS

SEQ ID NO: 16 - protein, 253 aa, Artificial Sequence
  analogous
MADYKDIVLTQSPSSLSASLGERVSLTCRSSQEISGYLGWLQQKPDGSIKRLIYAASTLDSGVPKRFSGSRSGSDYSLTISSLESEDFADYYCVQYASYPYTFGGGTKLEMKRGGGGSGGGGSGGGGSGGGGSQIQLQESGGGLVQPGGSMKLSCVASGFTASNYWMNWVRQSPEKGLDWVAEIRLKSNNYATHYAESVKGRFTISRDDSKSSVYLQMNNLRAEDTGIYYCTGYYYDSRYGYWGQGTTVTVSS

SEQ ID NO: 17 - protein, 253 aa, Artificial Sequence
  analogous
MADYKDIVLTQSPSSLSASLGERVSLTCRSSQEISGYLGWLQQKPDGSIKRLIYAASTLDSGVPKRFSGSRSGSDYSLTISSLESEDFADYYCVQYASYPYTFGGGTKLEMKRGGGGSGGGGSGGGGSGGGGSQIQLQESGGGLVQPGGSMKLSCVASGFTFSAYWMNWVRQSPEKGLDWVAEIRLKSNNYATHYAESVKGRFTISRDDSKSSVYLQMNNLRAEDTGIYYCTGYYYDSRYGYWGQGTTVTVSS

SEQ ID NO: 18 - protein, 253 aa, Artificial Sequence
  analogous
MADYKDIVLTQSPSSLSASLGERVSLTCRSSQEISGYLGWLQQKPDGSIKRLIYAASTLDSGVPKRFSGSRSGSDYSLTISSLESEDFADYYCVQYASYPYTFGGGTKLEMKRGGGGSGGGGSGGGGSGGGGSQIQLQESGGGLVQPGGSMKLSCVASGFTFSNYWMNWVRQSPEKGLDWVAEIRLKSNNYATHYAESVKGRFTISRDDSKSSVYLQMNNLRAEDTGIYYCTAYYYDSRYGYWGQGTTVTVSS

SEQ ID NO: 19 - protein, 253 aa, Artificial Sequence
  analogous
MADYKDIVLTQSPSSLSASLGERVSLTCRSSQEISGYLGWLQQKPDGSIKRLIYAASTLDSGVPKRFSGSRSGSDYSLTISSLESEDFADYYCVQYASYPYTFGGGTKLEMKRGGGGSGGGGSGGGGSGGGGSQIQLQESGGGLVQPGGSMKLSCVASGFTFSNYWMNWVRQSPEKGLDWVAEIRLKSNNYATHYAESVKGRFTISRDDSKSSVYLQMNNLRAEDTGIYYCTGFYYDSRYGYWGQGTTVTVSS

SEQ ID NO: 20 - protein, 253 aa, Artificial Sequence
  analogous
MADYKDIVLTQSPSSLSASLGERVSLTCRSSQEISGYLGWLQQKPDGSIKRLIYAASTLDSGVPKRFSGSRSGSDYSLTISSLESEDFADYYCVQYASYPYTFGGGTKLEMKRGGGGSGGGGSGGGGSGGGGSQIQLQESGGGLVQPGGSMKLSCVASGFTFSNYWMNWVRQSPEKGLDWVAEIRLKSNNYATHYAESVKGRFTISRDDSKSSVYLQMNNLRAEDTGIYYCTGYAYDSRYGYWGQGTTVTVSS

SEQ ID NO: 21 - protein, 253 aa, Artificial Sequence
  analogous
MADYKDIVLTQSPSSLSASLGERVSLTCRSSQEISGYLGWLQQKPDGSIKRLIYAASTLDSGVPKRFSGSRSGSDYSLTISSLESEDFADYYCVQYASYPYTFGGGTKLEMKRGGGGSGGGGSGGGGSGGGGSQIQLQESGGGLVQPGGSMKLSCVASGFTFSNYWMNWVRQSPEKGLDWVAEIRLKSNNYATHYAESVKGRFTISRDDSKSSVYLQMNNLRAEDTGIYYCTGYYADSRYGYWGQGTTVTVSS

SEQ ID NO: 22 - protein, 253 aa, Artificial Sequence
  analogous
MADYKDIVLTQSPSSLSASLGERVSLTCRSSQEISGYLGWLQQKPDGSIKRLIYAASTLDSGVPKRFSGSRSGSDYSLTISSLESEDFADYYCVQYASYPYTFGGGTKLEMKRGGGGSGGGGSGGGGSGGGGSQIQLQESGGGLVQPGGSMKLSCVASGFTFSNYWMNWVRQSPEKGLDWVAEIRLKSNNYATHYAESVKGRFTISRDDSKSSVYLQMNNLRAEDTGIYYCTGYYYASRYGYWGQGTTVTVSS

SEQ ID NO: 23 - protein, 253 aa, Artificial Sequence
  analogous
MADYKDIVLTQSPSSLSASLGERVSLTCRSSQEISGYLGWLQQKPDGSIKRLIYAASTLDSGVPKRFSGSRSGSDYSLTISSLESEDFADYYCVQYASYPYTFGGGTKLEMKRGGGGSGGGGSGGGGSGGGGSQIQLQESGGGLVQPGGSMKLSCVASGFTFSNYWMNWVRQSPEKGLDWVAEIRLKSNNYATHYAESVKGRFTISRDDSKSSVYLQMNNLRAEDTGIYYCTGYYYDSAYGYWGQGTTVTVSS

SEQ ID NO: 24 - protein, 253 aa, Artificial Sequence
  analogous
MADYKDIVLTQSPSSLSASLGERVSLTCRSSQEISGYLGWLQQKPDGSIKRLIYAASTLDSGVPKRFSGSRSGSDYSLTISSLESEDFADYYCVQYASYPYTFGGGTKLEMKRGGGGSGGGGSGGGGSGGGGSQIQLQESGGGLVQPGGSMKLSCVASGFTFSNYWMNWVRQSPEKGLDWVAEIRLKSNNYATHYAESVKGRFTISRDDSKSSVYLQMNNLRAEDTGIYYCTGYYYDSRAGYWGQGTTVTVSS

SEQ ID NO: 25 - protein, 253 aa, Artificial Sequence
  analogous
MADYKDIVLTQSPSSLSASLGERVSLTCRSSQEISGYLGWLQQKPDGSIKRLIYAASTLDSGVPKRFSGSRSGSDYSLTISSLESEDFADYYCVQYASYPYTFGGGTKLEMKRGGGGSGGGGSGGGGSGGGGSQIQLQESGGGLVQPGGSMKLSCVASGFTFSNYWMNWVRQSPEKGLDWVAEIRLKSNNYATHYAESVKGRFTISRDDSKSSVYLQMNNLRAEDTGIYYCTGYYYDSRYGAWGQGTTVTVSS